(19) United States
(12) Patent Application Publication
(10) Pub. No.: US 2024/0279278 A1
(43) Pub. Date: Aug. 22, 2024

(54) NMU RECEPTOR 2 AGONISTS

(71) Applicant: Boehringer Ingelheim International GmbH, Ingelheim am Rhein (DE)

(72) Inventors: Holger WAGNER, Biberach (DE); Angela BALJULS, Warthausen (DE); Albert BRENNAUER, Riedlingen (DE); Thomas Siegfried FOX, Biberach an der Riss (DE); Volker MACK, Ulm (DE); Stefan PETERS, Biberach an der Riss (DE); Daniel Paul TEUFEL, Hergatz (DE); Tina ZIMMERMANN, Mittelbiberach (DE)

(21) Appl. No.: 18/409,886

(22) Filed: Jan. 11, 2024

(30) Foreign Application Priority Data
Jan. 12, 2023 (EP) 23151260.9

(51) Int. Cl. C07K 7/06; A61P 3/04

(52) U.S. Cl. CPC C07K 7/06; A61P 3/04; A61K 38/00

(57) ABSTRACT

The present invention relates to NMU-8 analogues that are neuromedin U receptor 2 (NMUR2) agonists, processes for their preparation, pharmaceutical compositions comprising them and their use in therapy, particularly in the treatment or prevention of disease and/or condition associated with or modulated by NMUR2 activation such as obesity or obesity comorbidities.

### TECHNICAL FIELD

The present invention relates to NMU-8 analogues that are neuromedin U receptor 2 (NMUR2) agonists, processes for their preparation, pharmaceutical compositions containing them and their use in therapy, particularly in the treatment or prevention of diseases and/or conditions associated with or modulated by NMUR2 activation such as obesity and comorbidities thereof.

### BACKGROUND

Body overweight and obesity are defined as abnormal or excessive fat accumulation that presents a risk to health. In this regard, body overweight and obesity are major risk factors for several chronic diseases, including diabetes, particularly pre-diabetes and type 2 diabetes, cardiovascular diseases and certain types of cancer.

Despite long-standing efforts in the relative field, the number of overweight and obese patients is still growing. First line therapy for overweight and obese patients comprises diet and exercise but often are not sufficiently efficacious. Second line treatment options are bariatric surgery and pharmacotherapy. Available pharmacological treatments largely lack sufficient efficacy and/or tolerability, and only a limited number of approved therapies are available in the US and in Europe. Therefore, there is still a high medical need for more efficacious, safe, and tolerable treatment options.

Neuromedin U (hereinafter referred to as NMU) is a bioactive brain-gut peptide initially isolated from porcine spinal cord based on its ability to contract smooth-muscle.

Neuromedin U receptor 1 (NMUR1) and neuromedin U receptor 2 (NMUR2) are the major receptors of NMU and primarily expressed in the gastrointestinal tract and central nervous system respectively.

The sequence of the NMU is highly conserved across species, indicating the physiological relevance of the peptide. NMU peptides are C-terminal amidated peptides and the two major forms are composed of 25 amino acid residues (NMU-25) in human and porcine and 23 amino acid residues (NMU-23) in rodents. The human NMU-25 peptide consists of the following amino acid sequence C-terminal amidated: Phe-Arg-Val-Asp-Glu-Glu-Phe-Gln-Ser-Pro-Phe-Ala-Ser-Gln-Ser-Arg-Gly-Tyr-Phe-Leu-Phe-Arg-Pro-Arg-Asn-NH₂ (hNMU-25, SEQ ID NO: 89).

Shorter forms of the peptide consisting of the C-terminal eight amino acid residues have been identified as active cleavage products of NMU-25 in pig (named porcine NMU-8) and dog (named dog NMU-8). All mammalian species that have been examined share the identical C-terminal seven amino acid residues that are considered to be critical for bioactivity (Brighton et al, Pharmacol. Rev. 2004, 56, 231-248).

The C-terminal eight amino acid peptide from the human NMU-25 consist of: Tyr-Phe-Leu-Phe-Arg-Pro-Arg-Asn-NH₂ (hereinafter NMU-8, SEQ ID NO: 90).

The amino acid sequence of the C-terminal eight residues of the human NMU-25 is the same as that of the porcine NMU-8.

The role of NMU in the regulation of feeding and energy homeostasis is well documented (Teranishi et al, Int. J. Mol. Sci. 2021, 22, 4238). An intracerebroventricular administration of NMU attenuated food intake and feeding-associated behavior in rodents (Howard et al., Nature 2000, 406, 70-74) as well as increased locomotor activity and core body temperature (Nakazato et al., Biochem. Biophys. Res. Commun. 2000, 277, 191-194). A local injection of NMU to the paraventricular nucleus (PVN) or arcuate nucleus (ARC) has also been reported to exhibit an anorexic activity as in the case of its intracerebroventricular administration; therefore, major action sites of NMU are assumed to be PVN and ARC (Wren et al., Endocrinology 2002, 143, 4227-4234). Further, an intracerebroventricular administration of NMU antiserum into rat has shown to increase food intake, suggesting that the central NMU produces physiological effects that suppress food intake (Kojima et al., Biochem. Biophys. Res. Commun. 2000, 76, 435-438). It has also been reported that NMU knockout mice exhibited an obese phenotype (Hanada et al., Nat. Med. 2004, 10, 1067-1073), and that NMU overexpression in mice resulted in a lean phenotype with improved glucose homeostasis (Kowalski et al., J. Endocrinol. 2005, 185, 151-164). It was observed that acute and chronic peripheral administration of NMU in rodents dose-dependently reduced food intake and body weight (Peier et al., Endocrinology 2011, 152, 2644-2654). Moreover, recent studies in lean and DIO (diet-induced obese) mice have shown, that peripheral administration of peptidic NMUR2 agonists, with prolonged plasma stability due to N-terminal PEGylation, resulted in neuronal activation in the ARC and dorsal vagal complex, and led to inhibition of food intake and body weight loss (Kaisho et al., Int. J. Obesity 2017, 41, 1790-1797; Kanematsu-Yamaki et al., J. Med. Chem. 2017, 60, 6089-6097; Nagai et al, British J. Pharmacol. 2018, 175, 359-373). Additionally, mutations of the endogenous ligand for NMUR2 have been linked to obesity in human; missense mutations of NMU have been found in obese children and adults and two different coding variants have been associated with increased prevalence for overweight and obesity (Hainerová et al., Clin. Endocrinol Metab. 2006, 91, 5057-5063; Mitchel et al., British J. Pharmacol. 2009, 158, 87-103).

Thus, NMU and analogues thereof have been suggested to be effective in the regulation and treatment of metabolic disorders, such as obesity and diabetes.

However, the in vivo activity of NMU and NMU analogues is often compromised due to rapid degradation of the peptide after administration.

The unfavorable pharmacokinetic properties of native NMU (e.g. the short mean residence time (MRT) or half-life (t_{1/2}); the half-life of native NMU after subcutaneous injection is less than 5 min) were improved by conjugation with high-molecular weight polyethylene glycol (PEG) moiety (PEGylation) (Ingallinella et al., Bioorg. Med. Chem. 2012, 20, 4751-4759) or human serum albumin (HSA) (Neuner et al., J. Pept. Sci. 2014, 20, 7-19), showing in both cases long-lasting, potent anorexigenic, and glucose-normalizing activity.

NMU analogues comprising polyethylene glycol (PEG) modifications are well-known and studied. However, the NMU analogues comprising a PEG moiety show substantial disadvantages: the high-molecular weight PEG-chains are usually polydisperse causing significant problems in isolation, purification, and analytics of the relative NMU analogues, whereas the high stability of ether bonds in biological systems limit biodegradability which may lead to accumulation and vacuole formation in organs (Wang et al., Biomacromolecules 2020, 21, 3134-3139; Hong et al., J. Pharmacol. Toxicol. Methods 2020, 102, 106678).

Another strategy for modifying NMU or analogues thereof to provide increased and/or prolonged in vivo activity is by addition of a functional group, such as a lipid (lipidation) providing binding to serum albumin (for example wherein the lipid is a C₁₀-C₂₀ fatty acid or derivative thereof, such as a palmitate moiety). Bioorganic & Medicinal Chemistry Letters vol. 27 (2017) pages 4626-4629 discloses alkylated NMU-8 analogues including leucine at position 20 (Leu²⁰) and asparagine at position 25 (Asn²⁵) and teaches that the substitution of phenylalanine at position 19 (Phe¹⁹) and arginine at position 24 (Arg²⁴) play a role in conferring selectivity for hNMUR2 over hNMUR1 (i.e., Table 1: NMU-8 and compounds 13-16 versus compounds 17-21 of the above-mentioned Bioorganic & Medicinal Chemistry Letters).

WO2006/068326, WO2007/075439, WO2007/109135, WO2009/042053, WO2009/044918, WO2010/053830, WO2010/116752, WO2010/138343, WO2011/005611, WO 2012/050227 WO2015/095719, WO2018/135641, WO2018/135642, and EP2842965 A1 disclose PEGylated, lipidated or non-lipidated NMU analogue peptides having agonist activity at NMU receptor(s) for use in the treatment of metabolic disorders such as obesity and diabetes.

### Aim of the Invention

The administration of a NMU analogue compound having a short in vivo half-life must be repeated frequently, e.g. once per day. There is a disadvantage due to inconvenience to patients as these administrations are usually done subcutaneously. Such frequent administrations cause pain and discomfort to patients.

There is a need in the art for long acting NMU analogues selectively acting on the NMU receptor 2 that are substantially safe, well tolerated and exhibit an anorexigenic effect when administered peripherally.

Particularly, it would be desirable to improve the pharmacokinetic profile of NMU analogues prolonging the in vivo half-life (or mean residence time, MRT) as long-acting NMU analogues would increase the interval between two consecutive administrations of a medicament (e.g. once every week or even less frequent) improving the compliance of the patients and reducing the costs associated.

Moreover, it would be desirable to provide NMU analogues that are well tolerated, particularly with respect to gastrointestinal adverse effects, such as nausea, vomiting and/or diarrhoea.

For example, it would be desirable to provide NMUR2 agonists having a high selectivity over NMUR1. NMUR2 selectivity generally results in compounds showing a better tolerability in comparison to non-selective compounds. As NMUR1 is highly expressed in gastrointestinal tissues, it plays a role in gastrointestinal motility and NMUR1 agonists may provide adverse gastrointestinal effects such as diarrhoea (Dass et al., British J. Pharmacol. 2007, 150, 502-508; Nagai et al., British J. Pharmacol. 2018, 175, 359-373).

Furthermore, it would be desirable to provide NMUR2 agonists with suitable solubility and chemical/physical stability to be administered to a human being in need of therapy, particularly suitable for subcutaneous administration.

The present invention provides NMU-8 analogue compounds according to general formula (I)

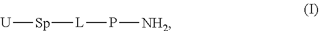

wherein U, Sp, L, and P are defined as hereinafter, that are effective agonists of NMUR2.

The NMU-8 analogues according to general formula (I) relate to peptides that have retained a high functional agonistic activity (EC₅₀) and binding affinity (Ki) towards NMUR2 (see table 4 and 5).

In addition to the agonistic property on NMUR2, the compounds of the present invention provide further advantageous properties as to be viable for human therapy, such as selectivity over NMUR1 (table 4 and 5, and tables 12A-B), prolonged in vivo half-life or mean residence time (table 6, and tables 13A-B), high solubility at a range of pH 4 to 8, preferably at a range of pH 6 to 8 (table 7), high chemical stability (table 8), and/or suitable physical stability (table 9).

Therefore, the compounds of the invention are viable for human therapy in the treatment of diseases and/or conditions that can be influenced by NMUR2 agonism having the potential to be administered less frequently and still be effective.

Accordingly, one aspect of the invention refers to compounds according to general formula (I) or salts thereof, preferably pharmaceutically acceptable salts thereof, as agonists of NMUR2.

A further aspect of the invention refers to compounds according to general formula (I) or salts thereof, preferably pharmaceutically acceptable salts, as agonists of NMUR2 with high selectivity over NMUR1.

A further aspect of the invention refers to compounds according to general formula (I) or salts thereof, preferably pharmaceutically acceptable salts, as agonists of NMUR2 with high selectivity over NMUR1 and prolonged in vivo half-life.

A further aspect of the invention refers to compounds according to general formula (I) or salts thereof, preferably pharmaceutically acceptable salts, as agonists of NMUR2 with high selectivity over NMUR1, prolonged in vivo half-life and adequate solubility at a range of pH 4 to 8, preferably at a range of pH 6 to 8, to be viable for human therapy, particularly for subcutaneous administration.

A further aspect of the invention refers to compounds according to general formula (I) or salts thereof, preferably pharmaceutically acceptable salts, as agonists of NMUR2 with high selectivity over NMUR1, prolonged in vivo half-life, adequate solubility at a range of pH 4 to 8, preferably at a range of pH 6 to 8, and suitable chemical and/or physical stability to be viable for human therapy, particularly for subcutaneous administration.

In a further aspect the invention relates to pharmaceutical compositions, preferably pharmaceutical composition suitable for subcutaneous administration, comprising at least one compound according to general formula (I), or pharmaceutically acceptable salts thereof, together with one or more inert adjuvant, diluent and/or carrier.

In a further aspect the invention relates to compounds according to general formula (I), pharmaceutically acceptable salts thereof, or pharmaceutical compositions thereof for therapeutic use in a method of prevention or treatment of disorders by activation of NMUR2 as disclosed hereinafter, such as obesity, body overweight condition and comorbidities thereof.

In a further aspect the invention relates to processes of manufacture of the compounds of the present invention or salts thereof, particularly pharmaceutically acceptable salts. The method may comprise the steps of synthesising the NMU-8 analogue by solid-phase or liquid-phase methodology, and optionally isolating and/or purifying the final product.

In a further aspect the invention relates to a compound of the present invention for use in a method of prevention or treatment of disorders by activation of NMUR2 as disclosed hereinafter, wherein the compound is administered as part of a combination therapy together with at least one further active agent suitable to treat disorders or conditions mentioned in the present application, preferably wherein the further active agent is an anti-obesity agent.

In a further aspect the invention relates to a pharmaceutical composition comprising a compound of the present invention and at least one further active agent suitable to treat disorders or conditions mentioned in the present application, preferably an anti-obesity agent, and use of the pharmaceutical composition in a method of prevention or treatment of disorders in the focus of the present invention.

Further aspects and embodiments of the present invention will become apparent from the disclosure below. Each aspect, embodiment or invention disclosed hereinbefore and hereinafter can be seen as separated invention or an invention by its own.

### GENERAL DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to.

### NMU-8 Analogues of the Present Invention

The terms “NMU-8 analogue of the present invention” or “compound of the present invention” refer to a compound of general formula (I)

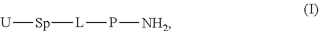

wherein
P is a peptide having an amino acids sequence of general formula (II)

(II)
(SEQ ID NO: 129)
Tyr-Phe-NMeLeu-Nle-X⁵-X⁶-Arg-Gly,

and
U, Sp, and L are defined as hereinafter.

The peptide P of general formula (II) corresponds to the NMU-8 amino acids sequence set forth in SEQ ID NO. 90, wherein two or more amino acid residues are substituted with amino acid residues which are each independently a residue of a naturally occurring amino acid or a non-naturally occurring amino acid as defined hereinafter. Thus, an NMU-8 analogue of the present invention according to general formula (I) is a peptide whose structure is related to NMU-8 set forth in SEQ ID NO 90.

Throughout this specification, amino acid positions in the peptide P of general formula (II) are numbered according to the corresponding position in the NMU-8 as shown below (the number shown in superscript after an amino acid represents the amino acid position number). Specifically, the position of tyrosine (Tyr) at the N-terminus of NMU-8 is regarded as position 1 and the position of asparagine (Asn) at the C-terminus is regarded as position 8.

(SEQ ID NO. 90)
Tyr¹-Phe²-Leu³-Phe⁴-Arg⁵-Pro⁶-Arg⁷-Asn⁸-NH₂

In peptide P of general formula (II) the amino acid residues at position 1, 2 and 7 are unchanged amino acid residues with respect to NMU-8, the amino acid residues at position 3, 4, and 8 are changed amino acid residues with respect to NMU-8, and the amino acid residues at position 5, and 6 can be each independently either changed or unchanged amino acid residues with respect to NMU-8.

Particularly, at position 3 the leucine (Leu) present in NMU-8 is changed to a N-methyl-leucine (NMeLeu) residue; at position 4 the phenylalanine (Phe) present in NMU-8 is changed to a norleucine (Nle) residue; at position 8 the asparagine (Asn) present in NMU-8 is changed to a glycine (Gly) residue.

Moreover, at position 5 and 6 the arginine (Arg) and the proline (Pro) present in NMU-8 respectively are each independently either changed to or unchanged amino acid residue according to the definitions of X⁵ and X⁶ as disclosed hereinafter.

Throughout the present specification, unless naturally occurring amino acids are referred to by their full name (e.g. alanine, arginine, etc.), they are designated by their conventional three-letter or single-letter abbreviations (e.g. Arg or R for arginine, etc.). In the case of certain less common or non-naturally occurring amino acids (i.e. amino acids other than the 20 encoded by the standard mammalian genetic code), unless they are referred to by their full name (e.g. y-glutamic acid, etc.), frequently employed three- or four-character codes are employed for residues thereof (e.g. g-Glu or y-Glu for (S)-y-glutamic acid, Dbu for (S)-2,4-diaminobutyric acid, etc.). Unless otherwise indicated, reference is made to the L-isomeric forms of the amino acids in question. Unless otherwise specified, compounds or residues containing chiral centers have the stereochemistry depicted.

The terms “NMU-8 analogue” or “compound” of the present invention refer to a peptide in which a “half-life extending group” (briefly U) is covalently attached via a spacer and a linker (briefly Sp and L, respectively) to the amino group of the N-terminal amino acid residue of the peptide P (i.e. Tyr), and in which a “NH₂ group” is attached to the C-terminal amino acid residue of the peptide P (i.e. Gly) to give a C-terminal amidated peptide.

The terms “NMU-8 analogue” or “compound” of the present invention refer to the compound of general formula (I) itself, as well as the compound of general formula (I) in ionized state (e.g. (de)protonated).

The meanings of the residues U, Sp, L, and P will be given hereinafter as embodiments of the invention (the indicate the attachment point to another moiety of the compound). Any and each of these definitions and embodiments may be combined with one another.

U is a half-life extending group (i.e. a lipophilic substituent) connected to a spacer Sp via an amide bond (i.e. U-Sp-), wherein the acyl group of U is linked to an amino group of Sp. U is selected from the group consisting of C16DA, C18DA and C20DA

wherein

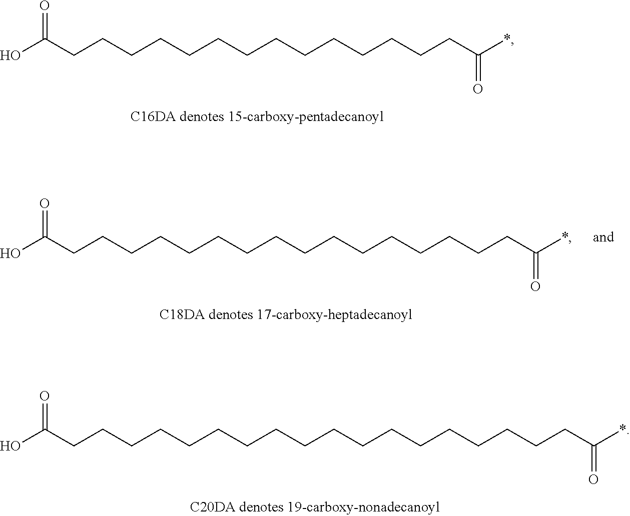

Without wishing to be bound by any particular theory, it is thought that such lipophilic substituents U (or the moiety U-Sp-) bind to albumin and other plasma components in the blood stream, thereby shielding the compound of the invention from renal filtration as well as enzymatic degradation and thus possibly enhancing the half-life of the compound in vivo.

Sp is a spacer according to the formula -(gGlu)ₙ-(Sar)ₘ- (SEQ ID NO: 149), wherein n is an integer number selected from the group consisting of 1, 2, 3 and 4, and m is any integer number from 3 to 15, preferably from 6 to 15 (i.e. selected from the group consisting of 3, 4, 5, 6, 7, 8, 9, 10, 11, 12, 13, 14, and 15), wherein gGlu and Sar are as defined hereinafter.

Most preferably, Sp is a spacer according to the formula -(gGlu)ₙ-(Sar)ₘ- (SEQ ID NO: 115), wherein n is an integer number selected from the group consisting of 1, 2, 3 and 4, and m is an integer number selected from the group consisting of 3, 6, 9, 12 and 15, wherein
gGlu (yGlu or isoGlu) refers to a residue of (S)-y-glutamic acid (also known as (2S)-2-aminopentane-dioic acid)

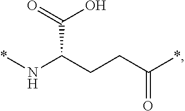

and
wherein
Sar (MeGly) refers to a sarcosine residue (also known as N-methylglycine or 2-(methylamino)acetic acid or [2-(methylamino)acetyl])

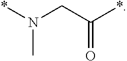

For example, the spacer “-(gGlu)ₙ-(Sar)ₘ- wherein n is 3 and m is 9” denotes the residue “-(gGlu)₃-(Sar)₉-”, that corresponds to “-gGlu-gGlu-gGlu-Sar-Sar-Sar-Sar-Sar-Sar-Sar-Sar-Sar-” and has the following structure:

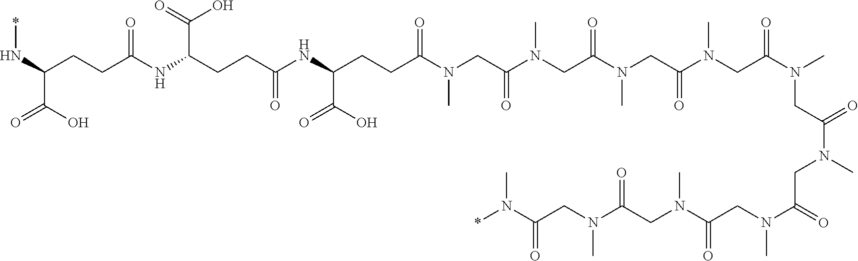 [SMILES: *N[C@@H](CCC(=O)N[C@@H](CCC(=O)N[C@@H](CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(*)C)C(=O)O)C(=O)O)C(=O)O]

In the spacer drawn above three residues of gGlu are connected to each other via amide bonds, the acyl group of a terminal gGlu residue is connected via an amide bond to the amino group of one terminal Sar residue, and nine Sar residues are connected to each other via amide bonds.

The spacer Sp is connected via an amide bond to U and via an amide bond to L (i.e. U-Sp-L), wherein the amino group of a terminal gGlu residue is connected via an amide bond to an acyl group of U, and the acyl group of a terminal Sar residue is connected via an amide bond to an amino group of L, i.e. U-gGlu-[ . . . ]-Sar-L.

L is a linker containing an amino group and an acyl group, wherein the amino group is connected via an amide bond to the acyl group of a terminal Sar residue of the spacer Sp, and the acyl group is connected via an amide bond to the amino group of the amino acid at sequence position 1 of the peptide P (i.e. -Sp-L-Tyr-).

L is selected from the group consisting of AEPipAc, PipAc, and AECMP,

wherein
AEPipAc refers to a residue of 2-[4-(2-aminoethyl)piperazin-1-yl]acetic acid

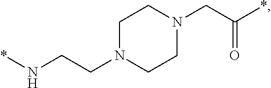

wherein
PipAc refers to a residue of 2-(piperazin-1-yl)acetic acid

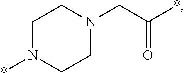

and
wherein
AECMP refers to a residue of 2-[1-(2-aminoethyl)piperidin-4-yl]acetic acid

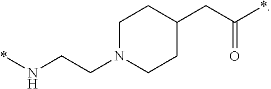

P is a peptide according to the following formula (II): Tyr-Phe-NMeLeu-Nle-X⁵—X⁶-Arg-Gly (SEQ ID NO: 129) (II).

The peptide P comprises both natural L-amino acids selected from the group consisting of Tyr (Y), Phe (F), Arg (R), Gly (G), His (H), Pro (P), and Lys (K), and unnatural amino acids selected from the group consisting of 2Pal, 3Pal, 4Pal, NMeLeu (NMeL), Nle, Dbu, Orn, NMeAla (NMeA), NMeArg (NMeR), hLys (hK), and bAla (bA),

wherein
2Pal refers to a residue of (2S)-2-pyridyl-alanine

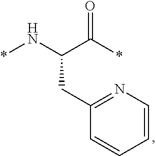 [SMILES: *N[C@@H](Cc1ccccn1)C(*)=O]

wherein
3Pal refers to a residue of (2S)-3-pyridyl-alanine

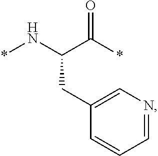 [SMILES: *N[C@@H](Cc1cccnc1)C(*)=O]

wherein
4Pal refers to a residue of (2S)-4-pyridyl-alanine

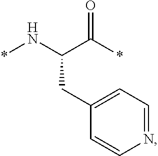 [SMILES: *N[C@@H](Cc1ccncc1)C(*)=O]

wherein
NMeLeu (NMeL) refers to a residue of (2S)—N-methyl-leucine

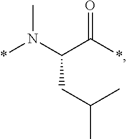

wherein
Nle (norleucine) refers to a residue of (S)-nor-leucine or (2S)-2-aminohexanoic acid

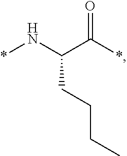

wherein
Dbu refers to a residue of (2S)-2,4-diaminobutyric acid or (2S)-2,4-diaminobutanoic acid

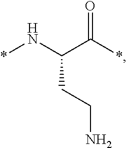

wherein
Orn (ornithine) refers to a residue of (2S)-2,5-diaminopentanoic acid

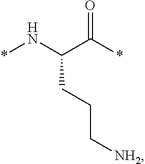 [SMILES: *N[C@@H](CCCN)C(*)=O]

wherein
NMeAla (NMeA) refers to a residue of (2S)—N-methyl-alanine

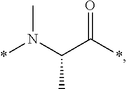

wherein
NMeArg (NMeR) refers to a residue of (2S)—N-methyl-arginine

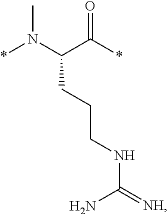 [SMILES: *C(=O)[C@H](CCCNC(=N)N)N(*)C]

wherein
hLys (hK, homo-lysine) refers to a residue of (2S)-2,6-diaminohexanoic acid

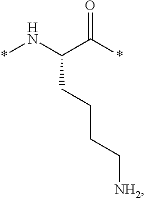 [SMILES: *N[C@@H](CCCCN)C(*)=O]

and
wherein
b-Ala (bA, beta-alanine) refers to a residue of 3-aminopropanoic acid

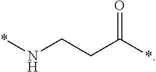

### Nomenclature of Compounds

As an example (compound No. 26 of table 3),
C18DA-gGlu-gGlu-gGlu-Sar-Sar-Sar-Sar-Sar-Sar-Sar-Sar-Sar-Sar-Sar-Sar-Sar-Sar-Sar-AEPipAc-Tyr-Phe-NMeLeu-Nle-Dbu-Pro-Arg-Gly-NH₂ (SEQ ID NO: 26) (i.e. C18DA-gGlu-gGlu-gGlu-Sar-Sar-Sar-Sar-Sar-Sar-Sar-Sar-Sar-Sar-Sar-Sar-Sar-Sar-Sar-AEPipAc-Y—F-NMeL-Nle-Dbu-P—R-G-NH₂ (SEQ ID NO: 26) or [17-carboxy-heptadecanoyl]-gGlu-gGlu-gGlu-Sar-Sar-Sar-Sar-Sar-Sar-Sar-Sar-Sar-Sar-Sar-Sar-Sar-Sar-Sar-AEPipAc-Y—F-NMeL-Nle-Dbu-P—R-G-NH₂ (SEQ ID NO: 26)),
   wherein

C18DA is connected via its a-carboxy group to a terminal gGlu residue;
gGlu-gGlu-gGlu (i.e. (2S)-2-[(4S)-4-[(4S)-4-amino-4-carboxybutanamido]-4-carboxybutanamido]pentanedioic acid) is connected via the amino group of a terminal gGlu residue to C18DA and via the y-carboxy group of the further terminal gGlu residue to a terminal Sar residue;
Sar-Sar-Sar-Sar-Sar-Sar-Sar-Sar-Sar-Sar-Sar-Sar-Sar-Sar-Sar (i.e. 2-{N-methyl-2-[N-methyl-2-(N-methyl-2-{N-methyl-2-[N-methyl-2-(N-methyl-2-{N-methyl-2-[N-methyl-2-(N-methyl-2-{N-methyl-2-[N-methyl-2-(N-methyl-2-{N-methyl-2-[N-methyl-2-
(methylamino)acetamido]acetamido}acetamido)acetamido]acetamido}acetamido)acetamido]acetamido}aceta mido)acetamido]acetamido}acetamido)acetamido]acetamido}acetic acid) is connected via the amino group of a terminal Sar residue to a terminal gGlu residue and via the carboxy group of the further terminal Sar residue to AEPipAc;
AEPipAc (i.e. 2-[4-(2-aminoethyl)piperazin-1-yl]acetic acid) is connected via its amino group to a terminal Sar residue and via its carboxy group to Tyr;
Tyr is connected via its amino group to AEPipAc and via its carboxy group to Phe;
Phe is connected via its amino group to Tyr and via its carboxy group to NMeLeu;
NMeLeu is connected via its amino group to Phe and via its carboxy group to Nle;
Nle is connected via its amino group to NMeLeu and via its carboxy group to Dbu;
Dbu is connected via its amino group to Nle and via its carboxy group to Pro;
Pro is connected via its amino group to Nle and via its carboxy group to Arg;
Arg is connected via its amino group to Pro and via its carboxy group to Gly;
Gly is connected via its amino group to Arg and via its carboxy group to NH₂;
and NH₂ is connected to the carboxy group of Gly;
completely defines the NMU-8 analogue of the following structure:

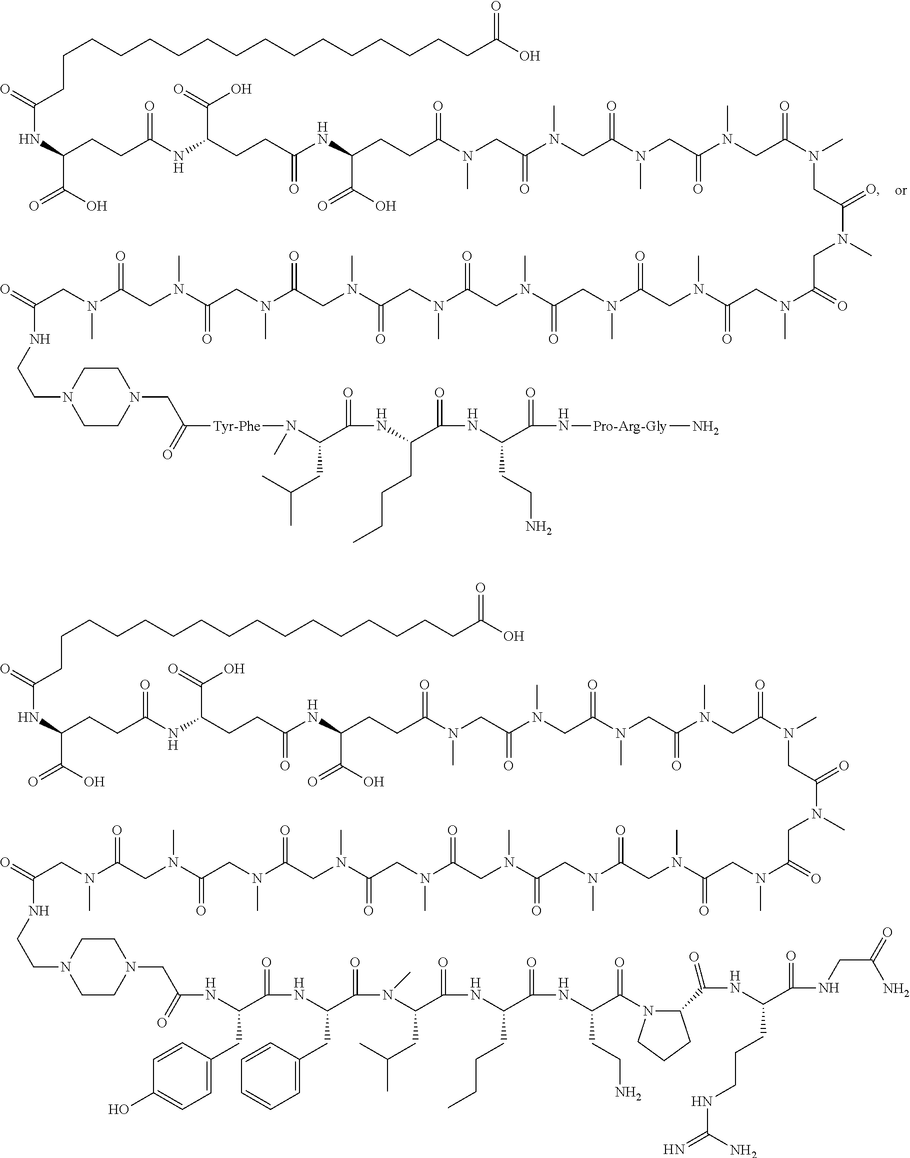

(SEQ ID NOS 26 and 26, respectively, in order of appearance). Alternatively, the same compound can be defined in the following way:

(SEQ ID NO: 26)
17-{[(1S)-3-{[(1S)-3-{[(1S)-3 -({[({[({[({[({
[({[({[({[({[({[({[({2-[4-(({[(1S)-1-{[(1S)-1-
{[(1S)-1-{[(1S)-1-{[(2S)-4-amino-1-[(2S)-2-
{[(1S)-4-carbamimidamido-1-[(carbamoylmethyl)
carbamoyl]butyl]carbamoyl}pyrrolidin-1-yl]-1-
oxobutan-2-yl]carbamoyl}pentyl]carbamoyl}-3-
methylbutyl](methyl) carbamoyl}-2-phenylethyl]
carbamoyl}-2-(4-hydroxyphenyl)ethyl]carbamoyl}
methyl)piperazin-1-yl]ethyl}carbamoyl)methyl]
(methyl)carbamoyl}methyl)(methyl)carbamoyl]
methyl}(methyl)carbamoyl)methyl](methyl)
carbamoyl}methyl)(methyl)carbamoyl]methyl}
(methyl)carbamoyl)methyl](methyl)carbamoyl}
methyl)(methyl)carbamoyl]methyl}(methyl)
carbamoyl)methyl](methyl)carbamoyl}methyl)
(methyl)carbamoyl]methyl}(methyl)carbamoyl)
methyl](methyl)carbamoyl}methyl)(methyl)
carbamoyl]methyl}(methyl)carbamoyl)-1-
carboxypropyl]carbamoyl}- 1-carboxypropyl]
carbamoyl}-1-carboxypropyl]carbamoyl}
heptadecanoic acid
or
(SEQ ID NO: 26)
2-[4-(2-(17-carboxyheptadecanoyl-L-γ-
glutamyl-L-γ-glutamyl-L-γ-glutamyl-N-
methylglycyl-N-methylglycyl-N-
methylglycyl-N-methylglycyl-N-methyl
glycyl-N-methylglycyl-N-methylglycyl-
N-methylglycyl-N-methylglycyl-N-
methylglycyl-N-methylglycyl-N-
methylglycyl-N-methylglycyl-N-methyl
glycyl-N-methylglycylamino)ethyl)
piperazin-1-yl]acetyl-
[NMeL³,Nle⁴,Dbu⁵,G⁸]NMU-8

### Additional Definitions

As used herein, the term “pharmaceutically acceptable salt” is intended to indicate a salt which is not harmful to a patient or subject to which the salt in question is administered. It may suitably be a salt chosen, e.g., among acid addition salts and basic salts. As used herein, “pharmaceutically acceptable salt” refers to derivatives of the disclosed analogues or compounds wherein the parent analogue or compound is modified by making acid or base salts thereof. Examples of acid addition salts include trifluoroacetic salts, chloride salts, citrate salts and acetate salts. Examples of basic salts include salts, where the cation is selected among alkali metal cations, such as sodium or potassium ions, alkaline earth metal cations, such as calcium or magnesium ions, as well as substituted ammonium ions, such as ions of the type N(R₁)(R₂)(R₃)(R₄)⁺, where R₁, R₂, R₃ and R₄ independently will typically designate hydrogen or optionally substituted C₁₋₆-alkyl. Other examples of pharmaceutically acceptable salts are described in “Remington's Pharmaceutical Sciences”, 17^{th} edition, Alfonso R. Gennaro (Ed.), Mark Publishing Company, Easton, PA, USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66: 1, pages 1-19 (1977).

The term “pharmaceutically acceptable adjuvant, diluent and/or carrier” includes any of the standard pharmaceutical adjuvant, diluent and/or carriers. Pharmaceutically acceptable adjuvant, diluent and/or carriers for therapeutic use are well known in the pharmaceutical art and are described, for example, in “Remington's Pharmaceutical Sciences”, 17th edition, Alfonso R. Gennaro (Ed.), Mark Publishing Company, Easton, PA, USA, 1985 (and more recent editions thereof). For example, sterile saline and phosphate-buffered saline at slightly acidic or physiological pH may be used. Suitable pH-buffering agents may be for example phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate, or mixtures thereof. The term further encompasses any adjuvant, diluent and/or carrier agents listed in the US Pharmacopeia for use in humans.

The term “agonist” as employed in the context of the invention refers to a substance that activates the receptor type in question, typically by binding to it (i.e. as a ligand).

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject in need of treatment may thus be a subject already afflicted with the disease or disorder in question. The term “treatment” includes inhibition or reduction of an increase in severity of a pathological state or symptoms (e.g. weight gain or hyperglycemia) relative to the absence of treatment, and is not necessarily meant to imply complete cessation of the relevant disease, disorder or condition.

The terms “prevention” and grammatical variants thereof (e.g., “prevented”, “preventing”, “prevent”) as employed in the present context refer to an approach for hindering or preventing the development of, or altering the pathology of, a condition, disease, or disorder. Accordingly, “prevention” may refer to prophylactic or preventive measures. For the purposes of this invention, beneficial or desired clinical results include, but are not limited to, prevention or slowing of symptoms, progression, or development of a disease, whether detectable or undetectable. A subject in need of “prevention” may thus be a subject not yet afflicted with the disease or disorder in question. The term “prevention” thus includes inhibiting or slowing the onset of disease relative to the absence of treatment and is not necessarily meant to imply permanent prevention of the relevant disease, disorder, or condition.

The terms “patient”, “subject,” and “individual” may be used interchangeably and refer to a human.

### DETAILED DESCRIPTION OF THE INVENTION

In a first aspect the present invention provides NMU-8 analogue compounds of general formula (I):

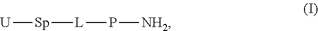

wherein
U is selected from the group Ug1 consisting of 15-carboxy-pentadecanoyl (briefly, C16DA), 17-carboxy-heptadecanoyl (briefly, C18DA) and 19-carboxy-nonadecanoyl (briefly, C20DA);
Sp is selected from the group Spg0 consisting of -(gGlu)ₙ-(Sar)ₘ- (SEQ ID Nos: 149), wherein
   n is selected from the group consisting of 1, 2, 3, and 4,
   m is any integer number from 3 to 15 (i.e. selected from the group consisting of 3, 4, 5, 6, 7, 8, 9, 10, 11, 12, 13, 14, and 15), preferably from 6 to 15, and
   wherein a terminal gGlu residue is linked to U and a terminal Sar residue is linked to L;
L is selected from the group Lg1 consisting of AEPipAc, PipAc, and AECMP; and
P is a peptide having an amino acids sequence of general formula (II)

(II)
(SEQ ID NO: 112)
Tyr-Phe-NMeLeu-Nle-X⁵-X⁶-Arg-Gly

wherein
X⁵ is selected from the group X⁵g1 consisting of Arg, Dbu, NMeArg, Orn, Lys, His, hLys, 2Pal, 3Pal, and 4Pal, and
X⁶ is selected from the group X⁶g1 consisting of Pro, NMeAla, bAla, and Gly;
or a salt thereof, preferably a pharmaceutically acceptable salt.

Preferably, in the first aspect the present invention provides NMU-8 analogue compounds of general formula (I):

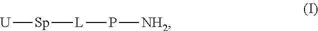

wherein
U is selected from the group Ug1 consisting of 15-carboxy-pentadecanoyl (briefly, C16DA), 17-carboxy-heptadecanoyl (briefly, C18DA) and 19-carboxy-nonadecanoyl (briefly, C20DA);
Sp is selected from the group Spg1 consisting of -(gGlu)ₙ-(Sar)ₘ- (SEQ ID NO: 115), wherein
   n is selected from the group consisting of 1, 2, 3, and 4, and
   m is selected from the group consisting of 6, 9, 12 and 15, and optionally 3 (i.e. 3, 6, 9, 12 and 15 or 6, 9, 12 and 15), and
   wherein a terminal gGlu residue is linked to U and a terminal Sar residue is linked to L;
L is selected from the group Lg1 consisting of AEPipAc, PipAc, and AECMP; and
P is a peptide having an amino acids sequence of general formula (II)

(II)
(SEQ ID NO: 112)
Tyr-Phe-NMeLeu-Nle-X⁵-X⁶-Arg-Gly

wherein
X⁵ is selected from the group X⁵g1 consisting of Arg, Dbu, NMeArg, Orn, Lys, His, hLys, 2Pal, 3Pal, and 4Pal, and
X⁶ is selected from the group X⁶g1 consisting of Pro, NMeAla, bAla, and Gly;
   or a salt thereof, preferably a pharmaceutically acceptable salt.

Unless otherwise stated, the groups, amino acid residues, and substituents, particularly U, Sp, L, P, X⁵, and X⁶, are defined as hereinbefore and hereinafter. Some preferred meanings of groups and amino acid residues of the compounds according to the invention will be given hereinafter.

In a further embodiment of the present invention U is selected from the group Ug2 consisting of C18DA and C20DA.

In a further embodiment of the present invention U is selected from the group Ug3 consisting of C18DA.

In a further embodiment of the present invention U is selected from the group Ug4 consisting of C20DA.

In a further embodiment of the present invention Sp is selected from the group Spg2 consisting of -(gGlu)ₙ-(Sar)ₘ- (SEQ ID NO: 116), wherein
n is 3 or 4, and
m is selected from the group consisting of 9, 12, and 15, preferably m is 12 or 15.

In a further embodiment of the present invention Sp is selected from the group Spg3 consisting of -(gGlu)ₙ-(Sar)ₘ-, wherein
n is 3, and
m is 12 or 15, preferably m is 15.

In a further embodiment of the present invention Sp is selected from the group Spg4 consisting of -(gGlu)ₙ-(Sar)ₘ- (SEQ ID NO: 118), wherein
n is 4, and
m is 12 or 15, preferably m is 15. In a further embodiment of the present invention L is selected from the group Lg2 consisting of AEPipAc, and PipAc.

In a further embodiment of the present invention L is selected from the group Lg3 consisting of AEPipAc.

In a further embodiment of the present invention P is a peptide having an amino acids sequence of general formula (II)

(II)
(SEQ ID NO: 113)
Tyr-Phe-NMeLeu-Nle-X⁵-X⁶-Arg-Gly

wherein
X⁵ is selected from the group X⁵g2 consisting of Arg, Dbu, NMeArg, Orn and Lys, preferably Arg, Dbu, NMeArg, and Orn; and
X⁶ is selected from the group X⁶g2 consisting of Pro, and NMeAla.

In a further embodiment of the present invention P is a peptide having an amino acids sequence of general formula (II)

(II)
(SEQ ID NO: 114)
Tyr-Phe-NMeLeu-Nle-X⁵-X⁶-Arg-Gly

wherein
X⁵ is selected from the group X⁵g3 consisting of Arg, Dbu, and Orn; and
X⁶ is selected from the group X⁶g3 consisting of Pro.

Each embodiment of the invention described herein may be taken alone or in combination with one or more other embodiments of the invention.

According to one preferred embodiment of the present invention, the peptide P of general formula (II) is selected from the group consisting of table 1.

TABLE 1

| | SEQ |
|---|---|
| | ID |
| Peptide P of formula (II) | NO |
| Tyr-Phe-NMeLeu-Nle-Arg-Pro-Arg-Gly | 91 |
| Tyr-Phe-NMeLeu-Nle-Orn-Pro-Arg-Gly | 92 |
| Tyr-Phe-NMeLeu-Nle-Dbu-Pro-Arg-Gly | 93 |
| Tyr-Phe-NMeLeu-Nle-Arg-NMeAla-Arg-Gly | 94 |
| Tyr-Phe-NMeLeu-Nle-Orn-NMeAla-Arg-Gly | 95 |
| Tyr-Phe-NMeLeu-Nle-Dbu-NMeAla-Arg-Gly | 96 |
| Tyr-Phe-NMeLeu-Nle-Lys-Pro-Arg-Gly | 97 |
| Tyr-Phe-NMeLeu-Nle-Lys-NMeAla-Arg-Gly | 98 |
| Tyr-Phe-NMeLeu-Nle-Orn-Gly-Arg-Gly | 99 |
| Tyr-Phe-NMeLeu-Nle-Orn-bAla-Arg-Gly | 100 |
| Tyr-Phe-NMeLeu-Nle-NMeArg-NMeAla-Arg-Gly | 101 |
| Tyr-Phe-NMeLeu-Nle-4Pal-Pro-Arg-Gly | 102 |
| Tyr-Phe-NMeLeu-Nle-2Pal-NMeAla-Arg-Gly | 103 |
| Tyr-Phe-NMeLeu-Nle-His-Pro-Arg-Gly | 104 |
| Tyr-Phe-NMeLeu-Nle-NMeArg-Pro-Arg-Gly | 105 |
| Tyr-Phe-NMeLeu-Nle-3Pal-Pro-Arg-Gly | 106 |
| Tyr-Phe-NMeLeu-Nle-His-NMeAla-Arg-Gly | 107 |
| Tyr-Phe-NMeLeu-Nle-2Pal-Pro-Arg-Gly | 108 |
| Tyr-Phe-NMeLeu-Nle-3Pal-NMeAla-Arg-Gly | 109 |
| Tyr-Phe-NMeLeu-Nle-4Pal-NMeAla-Arg-Gly | 110 |
| Tyr-Phe-NMeLeu-Nle-hLys-Pro-Arg-Gly | 111 |

According to another embodiment, the present invention provides NMU-8 analogues of general formula (I), wherein
U is selected from the group Ug2 consisting of C18DA and C20DA;
Sp is selected from the group Spg1 consisting of -(gGlu)ₙ-(Sar)ₘ- (SEQ ID NO: 115), wherein
   n is selected from the group consisting of 1, 2, 3, and 4, and
   m is selected from the group consisting of 6, 9, 12 and 15, and optionally 3;
L is selected from the group Lg2 consisting of AEPipAc and PipAc; and
P is a peptide of formula (II) selected from the group consisting of table 1.

According to another preferred embodiment, the present invention provides NMU-8 analogues of general formula (I), wherein
U is selected from the group Ug2 consisting of C18DA and C20DA;
Sp is selected from the group Spg2 consisting of -(gGlu)ₙ-(Sar)ₘ- (SEQ ID NO: 116), wherein
   n is selected from the group consisting of 3, and 4, and
   m is selected from the group consisting of 9, 12 and 15, preferably m is 12 or 15;
L is selected from the group Lg2 consisting of AEPipAc and PipAc; and
P is a peptide of formula (II) selected from the group consisting of table 1.

According to another preferred embodiment, the present invention provides NMU-8 analogues of general formula (I), wherein
U is selected from the group Ug3 consisting of C18DA;
Sp is selected from the group Spg2 consisting of -(gGlu)ₙ-(Sar)ₘ- (SEQ ID NO: 116), wherein
   n is selected from the group consisting of 3, and 4, and
   m is selected from the group consisting of 9, 12 and 15, preferably m is 12 or 15;
L is selected from the group Lg2 consisting of AEPipAc and PipAc; and
P is a peptide of formula (II) selected from the group consisting of table 1.

According to another preferred embodiment, the present invention provides NMU-8 analogue of general formula (I), wherein
U is selected from the group Ug4 consisting of C20DA;
Sp is selected from the group Spg2 consisting of -(gGlu)ₙ-(Sar)ₘ- (SEQ ID NO: 116), wherein
   n is selected from the group consisting of 3, and 4, and
   m is selected from the group consisting of 9, 12 and 15, preferably m is 12 or 15;
L is selected from the group Lg2 consisting of AEPipAc and PipAc; and
P is a peptide of formula (II) selected from the group consisting of table 1.

Further preferred embodiments of the compound of general formula (I) U-Sp-L-P—NH₂ are set forth as embodiments A-1 to A-25 in the following table 2 (salts and pharmaceutically salts thereof included), wherein the above-mentioned groups, amino acid residues and substituents definitions are used. For example, the entry ‘Ug2’ in column U at row A-1 means that in embodiment A-1 substituent U is selected from the definition designated Ug2. The same applies analogously to the other variables Sp, L, X⁵ and X⁶ incorporated in the general formula (I). Embodiments A-2 and A-6 to A-25 of table 2 are the more preferred embodiments, and A-6, A-11, A-19 and/or A-25 are the most preferred.

TABLE 2

| Embo- | | | | P (Tyr-Phe-NMeLeu-Nle-X5-X6-Arg-Gly) | | Construct |
|---|---|---|---|---|---|---|
| diment | U | Sp | L | (SEQ ID NO: 129) | NH₂ | SEQ ID NO: |
| A-1 | Ug2 | Spg1 | Lg1 | Tyr-Phe-NMeLeu-Nle-X⁵g1-X⁶g1-Arg-Gly | NH₂ | 130 |
| | | | | (SEQ ID NO: 112) | | |
| A-2 | Ug2 | Spg1 | Lg2 | Tyr-Phe-NMeLeu-Nle-X⁵g1-X⁶g1-Arg-Gly | NH₂ | 120 |
| | | | | (SEQ ID NO: 112) | | |
| A-3 | Ug2 | Spg1 | Lg3 | Tyr-Phe-NMeLeu-Nle-X⁵g1-X⁶g1-Arg-Gly | NH₂ | 131 |
| | | | | (SEQ ID NO: 112) | | |
| A-4 | Ug2 | Spg2 | Lg2 | Tyr-Phe-NMeLeu-Nle-X⁵g1-X⁶g1-Arg-Gly | NH₂ | 132 |
| | | | | (SEQ ID NO: 112) | | |
| A-5 | Ug3 | Spg2 | Lg2 | Tyr-Phe-NMeLeu-Nle-X⁵g1-X⁶g1-Arg-Gly | NH₂ | 133 |
| | | | | (SEQ ID NO: 112) | | |
| A-6 | Ug2 | Spg2 | Lg2 | Tyr-Phe-NMeLeu-Nle-X⁵g2-X⁶g2-Arg-Gly | NH₂ | 121 |
| | | | | (SEQ ID NO: 113) | | |
| A-7 | Ug2 | Spg2 | Lg3 | Tyr-Phe-NMeLeu-Nle-X⁵g2-X⁶g2-Arg-Gly | NH₂ | 134 |
| | | | | (SEQ ID NO: 113) | | |
| A-8 | Ug2 | Spg3 | Lg2 | Tyr-Phe-NMeLeu-Nle-X⁵g2-X⁶g2-Arg-Gly | NH₂ | 135 |
| | | | | (SEQ ID NO: 113) | | |
| A-9 | Ug2 | Spg4 | Lg3 | Tyr-Phe-NMeLeu-Nle-X⁵g2-X⁶g2-Arg-Gly | NH₂ | 136 |
| | | | | (SEQ ID NO: 113) | | |
| A-10 | Ug2 | Spg2 | Lg2 | Tyr-Phe-NMeLeu-Nle-X⁵g3-X⁶g3-Arg-Gly | NH₂ | 137 |
| | | | | (SEQ ID NO: 114) | | |
| A-11 | Ug2 | Spg2 | Lg3 | Tyr-Phe-NMeLeu-Nle-X⁵g3-X⁶g3-Arg-Gly | NH₂ | 122 |
| | | | | (SEQ ID NO: 114) | | |
| A-12 | Ug2 | Spg3 | Lg2 | Tyr-Phe-NMeLeu-Nle-X⁵g3-X⁶g3-Arg-Gly | NH₂ | 138 |
| | | | | (SEQ ID NO: 114) | | |
| A-13 | Ug2 | Spg4 | Lg3 | Tyr-Phe-NMeLeu-Nle-X⁵g3-X⁶g3-Arg-Gly | NH₂ | 139 |
| | | | | (SEQ ID NO: 114) | | |
| A-14 | Ug3 | Spg2 | Lg2 | Tyr-Phe-NMeLeu-Nle-X⁵g2-X⁶g2-Arg-Gly | NH₂ | 140 |
| | | | | (SEQ ID NO: 113) | | |
| A-15 | Ug3 | Spg2 | Lg3 | Tyr-Phe-NMeLeu-Nle-X⁵g2-X⁶g2-Arg-Gly | NH₂ | 141 |
| | | | | (SEQ ID NO: 113) | | |
| A-16 | U-g3 | Spg3 | Lg2 | Tyr-Phe-NMeLeu-Nle-X⁵g2-X⁶g2-Arg-Gly | NH₂ | 142 |
| | | | | (SEQ ID NO: 113) | | |
| A-17 | U-g3 | Spg2 | Lg2 | Tyr-Phe-NMeLeu-Nle-X⁵g3-X⁶g3-Arg-Gly | NH₂ | 143 |
| | | | | (SEQ ID NO: 114) | | |
| A-18 | U-g3 | Spg3 | Lg2 | Tyr-Phe-NMeLeu-Nle-X⁵g3-X⁶g3-Arg-Gly | NH₂ | 144 |
| | | | | (SEQ ID NO: 114) | | |
| A-19 | U-g3 | Spg3 | Lg3 | Tyr-Phe-NMeLeu-Nle-X⁵g3-X⁶g3-Arg-Gly | NH₂ | 123 |
| | | | | (SEQ ID NO: 114) | | |
| A-20 | Ug4 | Spg2 | Lg2 | Tyr-Phe-NMeLeu-Nle-X⁵g2-X⁶g2-Arg-Gly | NH₂ | 145 |
| | | | | (SEQ ID NO: 113) | | |
| A-21 | Ug4 | Spg2 | Lg3 | Tyr-Phe-NMeLeu-Nle-X⁵g2-X⁶g2-Arg-Gly | NH₂ | 146 |
| | | | | (SEQ ID NO: 113) | | |
| A-22 | Ug4 | Spg3 | Lg2 | Tyr-Phe-NMeLeu-Nle-X⁵g2-X⁶g2-Arg-Gly | NH₂ | 147 |
| | | | | (SEQ ID NO: 113) | | |
| A-23 | Ug4 | Spg4 | Lg3 | Tyr-Phe-NMeLeu-Nle-X⁵g2-X⁶g2-Arg-Gly | NH₂ | 126 |
| | | | | (SEQ ID NO: 113) | | |
| A-24 | Ug4 | Spg2 | Lg2 | Tyr-Phe-NMeLeu-Nle-X⁵g3-X⁶g3-Arg-Gly | NH₂ | 127 |
| | | | | (SEQ ID NO: 114) | | |
| A-25 | Ug4 | Spg4 | Lg3 | Tyr-Phe-NMeLeu-Nle-X⁵g3-X⁶g3-Arg-Gly | NH₂ | 124 |
| | | | | (SEQ ID NO: 114) | | |

Accordingly, for example embodiment A-6 of table 2 covers compounds of formula (I),
wherein
U is selected from the group Ug2 consisting of C18DA and C20DA;
Sp is selected from the group Spg2 consisting of -(gGlu)ₙ-(Sar)ₘ- (SEQ ID NO: 116), wherein
   n is selected from the group consisting of 3 and 4, and
   m is selected from the group consisting of 9, 12, and 15, preferably 12 and 15;
L is selected from the group Lg2 consisting of AEPipAc and PipAc; and
P is the peptide Tyr-Phe-NMeLeu-Nle-X⁵—X⁶-Arg-Gly (SEQ ID NO: 113), wherein
   X⁵ is selected from the group X⁵g2 consisting of Arg, Dbu, NMeArg, Orn, and Lys, preferably Arg, Dbu, NMeArg, and Orn, and
   X⁶ is selected from the group X⁵g2 consisting of Pro, and NMeAla;
or a salt thereof, preferably a pharmaceutically acceptable salt. Further preferred are the following compounds listed in table 3 or a salt thereof, preferably a pharmaceutically acceptable salt. In table 3 the Compound No. corresponds to the No. assigned in the Experimental section; any SEQ ID NO refers to a compound of formula (I) U-Sp-L-P—NH₂.

TABLE 3

| Com- | | SEQ. |
|---|---|---|
| pound | | ID |
| No. | U-Sp-L-P-NH₂ | NO |
| 1 | C20DA-gGlu₃-Sar₁₅-AEPipAc-Tyr-Phe-NMeLeu-Nle-Orn-Pro-Arg-Gly-NH₂ | 1 |
| 2 | C18DA-gGlu₄-Sar₁₂-AEPipAc-Tyr-Phe-NMeLeu-Nle-Arg-Pro-Arg-Gly-NH₂ | 2 |
| 3 | C20DA-gGlu₄-Sar₁₅-AEPipAc-Tyr-Phe-NMeLeu-Nle-Orn-Pro-Arg-Gly-NH₂ | 3 |
| 4 | C20DA-gGlu₄-Sar₁₅-AEPipAc-Tyr-Phe-NMeLeu-Nle-Dbu-Pro-Arg-Gly-NH₂ | 4 |
| 5 | C18DA-gGlu₄-Sar₁₂-AEPipAc-Tyr-Phe-NMeLeu-Nle-Orn-Pro-Arg-Gly-NH₂ | 5 |
| 6 | C20DA-gGlu₃-Sar₁₂-AEPipAc-Tyr-Phe-NMeLeu-Nle-Arg-Pro-Arg-Gly-NH₂ | 6 |
| 7 | C18DA-gGlu₃-Sar₁₅-AEPipAc-Tyr-Phe-NMeLeu-Nle-Arg-Pro-Arg-Gly-NH₂ | 7 |
| 8 | C20DA-gGlu₃-Sar₁₂-AEPipAc-Tyr-Phe-NMeLeu-Nle-Dbu-Pro-Arg-Gly-NH₂ | 8 |
| 9 | C20DA-gGlu₃-Sar₁₅-AEPipAc-Tyr-Phe-NMeLeu-Nle-Arg-Pro-Arg-Gly-NH₂ | 9 |
| 10 | C20DA-gGlu₃-Sar₁₅-AEPipAc-Tyr-Phe-NMeLeu-Nle-Arg-NMeAla-Arg-Gly-NH₂ | 10 |
| 11 | C18DA-gGlu₃-Sar₁₅-AEPipAc-Tyr-Phe-NMeLeu-Nle-Arg-NMeAla-Arg-Gly-NH₂ | 11 |
| 12 | C18DA-gGlu₃-Sar₁₂-AEPipAc-Tyr-Phe-NMeLeu-Nle-Arg-NMeAla-Arg-Gly-NH₂ | 12 |
| 13 | C20DA-gGlu₄-Sar₁₂-AEPipAc-Tyr-Phe-NMeLeu-Nle-Orn-NMeAla-Arg-Gly-NH₂ | 13 |
| 14 | C20DA-gGlu₄-Sar₁₅-AEPipAc-Tyr-Phe-NMeLeu-Nle-Orn-NMeAla-Arg-Gly-NH₂ | 14 |
| 15 | C20DA-gGlu₄-Sar₁₂-AEPipAc-Tyr-Phe-NMeLeu-Nle-Dbu-Pro-Arg-Gly-NH₂ | 15 |
| 16 | C20DA-gGlu₄-Sar₁₅-AEPipAc-Tyr-Phe-NMeLeu-Nle-Dbu-NMeAla-Arg-Gly-NH₂ | 16 |
| 17 | C20DA-gGlu₄-Sar₁₅-AEPipAc-Tyr-Phe-NMeLeu-Nle-Lys-Pro-Arg-Gly-NH₂ | 17 |
| 18 | C20DA-gGlu₄-Sar₁₅-AEPipAc-Tyr-Phe-NMeLeu-Nle-Lys-NMeAla-Arg-Gly-NH₂ | 18 |
| 19 | C20DA-gGlu₄-Sar₁₂-AEPipAc-Tyr-Phe-NMeLeu-Nle-Lys-NMeAla-Arg-Gly-NH₂ | 19 |
| 20 | C18DA-gGlu₃-Sar₁₂-AEPipAc-Tyr-Phe-NMeLeu-Nle-Orn-NMeAla-Arg-Gly-NH₂ | 20 |
| 21 | C18DA-gGlu₃-Sar₁₅-AEPipAc-Tyr-Phe-NMeLeu-Nle-Orn-Pro-Arg-Gly-NH₂ | 21 |
| 22 | C18DA-gGlu₃-Sar₁₅-AEPipAc-Tyr-Phe-NMeLeu-Nle-Orn-NMeAla-Arg-Gly-NH₂ | 22 |
| 23 | C18DA-gGlu₃-Sar₁₅-AEPipAc-Tyr-Phe-NMeLeu-Nle-Dbu-NMeAla-Arg-Gly-NH₂ | 23 |
| 24 | C18DA-gGlu₃-Sar₉-AEPipAc-Tyr-Phe-NMeLeu-Nle-Dbu-Pro-Arg-Gly-NH₂ | 24 |
| 25 | C18DA-gGlu₃-Sar₁₂-AEPipAc-Tyr-Phe-NMeLeu-Nle-Arg-Pro-Arg-Gly-NH₂ | 25 |
| 26 | C18DA-gGlu₃-Sar₁₅-AEPipAc-Tyr-Phe-NMeLeu-Nle-Dbu-Pro-Arg-Gly-NH₂ | 26 |
| 27 | C18DA-gGlu₃-Sar₁₂-AEPipAc-Tyr-Phe-NMeLeu-Nle-Dbu-Pro-Arg-Gly-NH₂ | 27 |
| 28 | C18DA-gGlu₃-Sar₉-AEPipAc-Tyr-Phe-NMeLeu-Nle-Dbu-NMeAla-Arg-Gly-NH₂ | 28 |
| 29 | C20DA-gGlu₃-Sar₁₅-AEPipAc-Tyr-Phe-NMeLeu-Nle-Orn-Gly-Arg-Gly-NH₂ | 29 |
| 30 | C20DA-gGlu₃-Sar₁₅-AEPipAc-Tyr-Phe-NMeLeu-Nle-Orn-bAla-Arg-Gly-NH₂ | 30 |
| 31 | C20DA-gGlu₄-Sar₁₂-AEPipAc-Tyr-Phe-NMeLeu-Nle-Dbu-NMeAla-Arg-Gly-NH₂ | 31 |
| 32 | C20DA-gGlu₄-Sar₁₂-AEPipAc-Tyr-Phe-NMeLeu-Nle-Lys-Pro-Arg-Gly-NH₂ | 32 |
| 33 | C18DA-gGlu₃-Sar₉-AEPipAc-Tyr-Phe-NMeLeu-Nle-Orn-Pro-Arg-Gly-NH₂ | 33 |
| 34 | C20DA-gGlu₃-Sar₁₅-AEPipAc-Tyr-Phe-NMeLeu-Nle-Orn-NMeAla-Arg-Gly-NH₂ | 34 |
| 35 | C18DA-gGlu₃-Sar₁₂-AEPipAc-Tyr-Phe-NMeLeu-Nle-NMeArg-Pro-Arg-Gly-NH₂ | 35 |
| 36 | C20DA-gGlu₃-Sar₁₅-AEPipAc-Tyr-Phe-NMeLeu-Nle-NMeArg-NMeAla-Arg-Gly-NH₂ | 36 |
| 37 | C18DA-gGlu₃-Sar₁₂-AEPipAc-Tyr-Phe-NMeLeu-Nle-NMeArg-NMeAla-Arg-Gly-NH₂ | 37 |
| 38 | C18DA-gGlu₃-Sar₁₂-AEPipAc-Tyr-Phe-NMeLeu-Nle-Dbu-NMeAla-Arg-Gly-NH₂ | 38 |
| 39 | C20DA-gGlu₄-Sar₁₂-AEPipAc-Tyr-Phe-NMeLeu-Nle-Arg-Pro-Arg-Gly-NH₂ | 39 |
| 40 | C20DA-gGlu₄-Sar₁₂-AEPipAc-Tyr-Phe-NMeLeu-Nle-Orn-Pro-Arg-Gly-NH₂ | 40 |
| 41 | C18DA-gGlu₄-Sar₁₂-AEPipAc-Tyr-Phe-NMeLeu-Nle-Dbu-Pro-Arg-Gly-NH₂ | 41 |
| 42 | C20DA-gGlu₃-Sar₁₅-AEPipAc-Tyr-Phe-NMeLeu-Nle-Dbu-Pro-Arg-Gly-NH₂ | 42 |
| 43 | C20DA-gGlu₃-Sar₁₅-AEPipAc-Tyr-Phe-NMeLeu-Nle-Dbu-NMeAla-Arg-Gly-NH₂ | 43 |
| 44 | C18DA-gGlu₂-Sar₉-AEPipAc-Tyr-Phe-NMeLeu-Nle-Orn-NMeAla-Arg-Gly-NH₂ | 44 |
| 45 | C18DA-gGlu₃-Sar₁₅-AEPipAc-Tyr-Phe-NMeLeu-Nle-₄Pal-Pro-Arg-Gly-NH₂ | 45 |
| 46 | C18DA-gGlu₃-Sar₁₅-AEPipAc-Tyr-Phe-NMeLeu-Nle-Lys-Pro-Arg-Gly-NH₂ | 46 |
| 47 | C18DA-gGlu₃-Sar₁₅-AEPipAc-Tyr-Phe-NMeLeu-Nle-Lys-NMeAla-Arg-Gly-NH₂ | 47 |
| 48 | C18DA-gGlu₃-Sar₁₅-AEPipAc-Tyr-Phe-NMeLeu-Nle-₂Pal-NMeAla-Arg-Gly-NH₂ | 48 |
| 49 | C20DA-gGlu₃-Sar₁₅-AEPipAc-Tyr-Phe-NMeLeu-Nle-Lys-Pro-Arg-Gly-NH₂ | 49 |
| 50 | C20DA-gGlu₃-Sar₁₅-AEPipAc-Tyr-Phe-NMeLeu-Nle-Lys-NMeAla-Arg-Gly-NH₂ | 50 |
| 51 | C18DA-gGlu₃-Sar₁₅-AEPipAc-Tyr-Phe-NMeLeu-Nle-His-Pro-Arg-Gly-NH₂ | 51 |
| 52 | C18DA-gGlu₃-Sar₁₅-AEPipAc-Tyr-Phe-NMeLeu-Nle-NMeArg-Pro-Arg-Gly-NH₂ | 52 |
| 53 | C18DA-gGlu₃-Sar₁₅-AEPipAc-Tyr-Phe-NMeLeu-Nle-NMeArg-NMeAla-Arg-Gly-NH₂ | 53 |
| 54 | C18DA-gGlu₃-Sar₁₅-AEPipAc-Tyr-Phe-NMeLeu-Nle-₃Pal-Pro-Arg-Gly-NH₂ | 54 |
| 55 | C18DA-gGlu₃-Sar₁₅-AEPipAc-Tyr-Phe-NMeLeu-Nle-His-NMeAla-Arg-Gly-NH₂ | 55 |
| 56 | C18DA-gGlu₃-Sar₁₅-AEPipAc-Tyr-Phe-NMeLeu-Nle-₂Pal-Pro-Arg-Gly-NH₂ | 56 |
| 57 | C18DA-gGlu₃-Sar₁₅-AEPipAc-Tyr-Phe-NMeLeu-Nle-₃Pal-NMeAla-Arg-Gly-NH₂ | 57 |
| 58 | C18DA-gGlu₃-Sar₁₅-AEPipAc-Tyr-Phe-NMeLeu-Nle-₄Pal-NMeAla-Arg-Gly-NH₂ | 58 |
| 59 | C20DA-gGlu₃-Sar₁₂-AEPipAc-Tyr-Phe-NMeLeu-Nle-Dbu-NMeAla-Arg-Gly-NH₂ | 59 |
| 60 | C20DA-gGlu₃-Sar₉-AEPipAc-Tyr-Phe-NMeLeu-Nle-Dbu-Pro-Arg-Gly-NH₂ | 60 |
| 61 | C20DA-gGlu₃-Sar₉-AEPipAc-Tyr-Phe-NMeLeu-Nle-Dbu-NMeAla-Arg-Gly-NH₂ | 61 |
| 62 | C20DA-gGlu₃-Sar₉-AEPipAc-Tyr-Phe-NMeLeu-Nle-Arg-NMeAla-Arg-Gly-NH₂ | 62 |
| 63 | C20DA-gGlu₃-Sar₁₂-AEPipAc-Tyr-Phe-NMeLeu-Nle-Arg-NMeAla-Arg-Gly-NH₂ | 63 |
| 65 | C20DA-gGlu₃-Sar₉-AEPipAc-Tyr-Phe-NMeLeu-Nle-Arg-Pro-Arg-Gly-NH₂ | 65 |
| 66 | C18DA-gGlu₂-Sar₉-AEPipAc-Tyr-Phe-NMeLeu-Nle-Arg-NMeAla-Arg-Gly-NH₂ | 66 |
| 67 | C18DA-gGlu₂-Sar₉-AEPipAc-Tyr-Phe-NMeLeu-Nle-Arg-Pro-Arg-Gly-NH₂ | 67 |
| 68 | C18DA-gGlu₃-Sar₆-AEPipAc-Tyr-Phe-NMeLeu-Nle-Arg-Pro-Arg-Gly-NH₂ | 68 |
| 69 | C18DA-gGlu₃-Sar₁₂-AEPipAc-Tyr-Phe-NMeLeu-Nle-Orn-Pro-Arg-Gly-NH₂ | 69 |
| 70 | C18DA-gGlu₃-Sar₉-AEPipAc-Tyr-Phe-NMeLeu-Nle-Orn-NMeAla-Arg-Gly-NH₂ | 70 |
| 71 | C18DA-gGlu₂-Sar₆-AEPipAc-Tyr-Phe-NMeLeu-Nle-Arg-Pro-Arg-Gly-NH₂ | 71 |
| 72 | C18DA-gGlu₃-Sar₉-AEPipAc-Tyr-Phe-NMeLeu-Nle-Arg-Pro-Arg-Gly-NH₂ | 72 |
| 73 | C18DA-gGlu₃-Sar₉-AEPipAc-Tyr-Phe-NMeLeu-Nle-Arg-NMeAla-Arg-Gly-NH₂ | 73 |
| 74 | C18DA-gGlu₃-Sar₉-AEPipAc-Tyr-Phe-NMeLeu-Nle-hLys-Pro-Arg-Gly-NH₂ | 74 |
| 75 | C18DA-gGlu₃-Sar₁₅-PipAc-Tyr-Phe-NMeLeu-Nle-Arg-Pro-Arg-Gly-NH₂ | 75 |
| 76 | C18DA-gGlu₃-Sar₁₅-PipAc-Tyr-Phe-NMeLeu-Nle-Dbu-Pro-Arg-Gly-NH₂ | 76 |
| 77 | C18DA-gGlu₃-Sar₁₂-PipAc-Tyr-Phe-NMeLeu-Nle-Arg-Pro-Arg-Gly-NH₂ | 77 |
| 78 | C20DA-gGlu₃-Sar₁₂-PipAc-Tyr-Phe-NMeLeu-Nle-Arg-Pro-Arg-Gly-NH₂ | 78 |
| 79 | C18DA-gGlu₃-Sar₁₅-PipAc-Tyr-Phe-NMeLeu-Nle-Dbu-NMeAla-Arg-Gly-NH₂ | 79 |
| 80 | C20DA-gGlu₃-Sar₁₅-PipAc-Tyr-Phe-NMeLeu-Nle-Orn-Pro-Arg-Gly-NH₂ | 80 |

The NMU-8 analogues of the present invention according to general formula (I), preferably compounds of table 3, are able to bind to the human NMU receptor 2 (hNMUR2) (see table 4).

Binding to biological receptors can be measured by appropriate assays known in the art. For instance, binding of NMU-8 analogues of the present invention to the human NMU receptor 2 (hNMUR2) can be evaluated by radio-ligand binding competition assays, e.g. as described in Example 1, below.

In some embodiments of compounds of the present invention, the binding affinity (Ki) towards hNMUR2 is below 1000 nM (e.g. 0.1 nM to 1000 nM).

In some embodiments of compounds of the present invention, the binding affinity (Ki) towards hNMUR2 is below 250 nM (e.g. 0.1 nM to 250 nM).

In some embodiments of compounds of the present invention, the binding affinity (Ki) towards hNMUR2 is below 100 nM (e.g. 0.1 nM to 100 nM).

In some embodiments of compounds of the present invention, the binding affinity (Ki) towards hNMUR2 is below 50 nM (e.g. 0.1 nM to 50 nM).

In some embodiments of compounds of the present invention, the binding affinity (Ki) towards hNMUR2 is below nM (e.g. 0.1 nM to 25 nM).

The NMU-8 analogues of the present invention activate the human NMU receptor 2 (hNMUR2), i.e. they are NMU receptor 2 agonists (see table 5).

In general, it is preferred to use a biological assay which measures intracellular signalling caused by binding of the compound to the relevant receptor. Activation of the NMU receptor by compounds of the invention (which behave as agonists of the receptor) increases myo-inositol-1-phosphate (IP1) concentrations effecting intracellular signalling pathways. Thus, increase of IP1 or any other suitable parameter in cells expressing the receptor can be used to monitor agonist activity towards the receptor. The skilled person will be aware of suitable assay formats, and examples are provided below.

EC₅₀ values may be used as a numerical measure of agonist potency at a given receptor. An EC₅₀ value is a measure of the concentration of a compound required to achieve half of that compound's maximal activity in a particular assay, e.g. in the assay as described in Example 3, below.

In some embodiments of compounds of the present invention, the EC₅₀ towards hNMUR2 is below 200 nM (e.g. 0.01 nM to 200 nM).

In some embodiments of compounds of the present invention, the EC₅₀ towards hNMUR2 is below 100 nM (e.g. 0.01 nM to 100 nM).

In some embodiments of compounds of the present invention, the EC₅₀ towards hNMUR2 is below 50 nM (e.g. 0.01 nM to 50 nM).

In some embodiments of compounds of the present invention, the EC₅₀ towards hNMUR2 is below 15 nM (e.g. 0.01 nM to 10 nM).

In some embodiments of compounds of the present invention, the EC₅₀ towards hNMUR2 is below 5 nM (e.g. 0.01 nM to 5 nM).

In some embodiments of compounds of the present invention, the EC₅₀ towards hNMUR2 is below 2 nM (e.g. 0.01 nM to 2 nM).

The NMU-8 analogues of the present invention are able to activate the human NMU receptor 2 (hNMUR2) in the presence of human serum albumin, the main protein present in human plasma and known to bind lipidated peptides (see table 5). Activity of described NMU-8 analogues in the presence of human plasma is therefore an important prerequisite for in vivo activity. Activation of the NMU receptor 2 in the presence of human plasma by compounds of the present invention (which behave as agonists of the receptor) increases myo-inositol-1-phosphate (IP1) concentrations effecting intracellular signalling pathways. Thus, increase of IP1 or any other suitable parameter in the presence of human plasma in cells expressing the receptor can be used to monitor agonist activity towards the receptor. The skilled person will be aware of suitable assay formats, and examples are provided below.

For instance, for NMU-8 analogues of the present invention the EC₅₀ value to the human NMU receptor 2 (hNMUR2) in the presence of human plasma can be evaluated by the assay as described in Example 4, below.

In some embodiments of compounds of the present invention, the EC₅₀ towards hNMUR2 in human plasma is below 1000 nM (e.g. 0.01 nM to 150 nM).

In some embodiments of compounds of the present invention, the EC₅₀ towards hNMUR2 is below 500 nM (e.g. 0.01 nM to 500 nM).

In some embodiments of compounds of the present invention, the EC₅₀ towards hNMUR2 is below 100 nM (e.g. 0.01 nM to 100 nM).

In some embodiments of compounds of the present invention, the EC₅₀ towards hNMUR2 is below 50 nM (e.g. 0.01 nM to 50 nM).

In some embodiments of compounds of the present invention, the EC₅₀ towards hNMUR2 is below 10 nM (e.g. 0.01 nM to 10 nM).

The NMU-8 analogues of the invention show a high selectivity with regard to binding affinity and to activation for the human NMU receptor 2 (hNMUR2) over human NMU receptor 1 (hNMUR1), as disclosed below in table 4 (see Example 2) and in table 5 (see Example 3), respectively.

The compounds of the present invention have favourable pharmacokinetic properties. In this regard, in some embodiments of the invention, the in-vivo mean residence time (MRT) after intravenous (iv) application of the NMU-8 analogues of the invention is at least 3 hours in the mouse (e.g. comprised from 3 hours to 50 hours; see measurement described in Example 5, table 6). In some embodiments, the in-vivo mean residence time is at least 5 hours in the mouse (e.g. comprised from 5 hours to 50 hours). In some embodiments, the in-vivo mean residence time is at least 7 hours in the mouse (e.g. comprised from 7 hours to 50 hours). In some embodiments, the in-vivo mean residence time is at least 10 hours in the mouse (e.g. comprised from 10 hours to 50 hours). In some embodiments, the in-vivo half-life is at least 15 hours in the mouse (e.g. comprised from hours to 50 hours, for example about 16 hs (hours), 17 hs, 18 hs, 19 hs, 20 hs, 21 hs, 22 hs, 23 hs, 24 hs, hs, 26 hs, 27 hs, 28 hs, 29 hs, 30 hs, 31 hs, 32 hs, 33 hs, 34 hs, 35 hs, 36 hs, 37 hs, 38 hs, 39 hs, 40 hs, 41 hs, 42 hs, 43 hs, 44 hs, 45 hs, 46 hs, 47 hs, 48 hs, and 49 hs).

The compounds of the present invention of general formula (I) are soluble at pH-values compatible with subcutaneous application to a patient. There are several techniques known to the skilled person in the art how to determine solubility. One such experiment is described below under Example 6 and the relative data in table 7.

In some embodiments of compounds of the present invention, the solubility is above 0.2 mg/ml at a pH 4 to 8, preferably at a pH 6 to 8, more preferably around pH 7.4 (e.g. 0.2 mg/ml to 50 mg/ml).

In some embodiments of compounds of the present invention, the solubility is above 1 mg/ml at a pH 4 to 8, preferably at a pH 6 to 8, more preferably around pH 7.4 (e.g. 1 mg/ml to 50 mg/ml).

In some embodiments of compounds of the present invention, the solubility is above 5 mg/ml at a pH 4 to 8, preferably at a pH 6 to 8, more preferably around pH 7.4 (e.g. 5 mg/ml to 50 mg/ml).

The compounds of the present invention of general formula (I) are chemically stable in a buffer system compatible with subcutaneous application to a patient. There are several techniques known to the skilled person in the art how to determine chemical stability. One such experiment is described below under Example 7 and the relative data in table 8, describing chemical stability by the percentage of degradation of the given compound at a certain temperature over time.

In some embodiments of compounds of the present invention, the percentage of degradation is below 20%.

In some embodiments of compounds of the present invention, the percentage of degradation is below 15%.

In some embodiments of compounds of the present invention, the percentage of degradation is below 10%.

In some embodiments of compounds of the present invention, the percentage of degradation is below 5%.

In some embodiments of compounds of the present invention, the percentage of degradation is below 1%.

The compounds of the present invention of general formula (I) are physically stable in a buffer system compatible with subcutaneous application to a patient. There are several techniques known to the skilled person in the art how to determine physical stability. One such experiment is described below under Example 8 and the relative data in table 9. The data of table 9 show no detection of fibrillation at the experimental condition for compounds of the present invention.

### Pharmaceutical Compositions

The invention also refers to pharmaceutical compositions comprising at least one compound of general formula (I), or a pharmaceutically acceptable salt thereof, together with at least one pharmaceutically acceptable adjuvant, diluent and/or carrier.

In a further aspect the invention refers to the pharmaceutical compositions for use in a method of preventing or treating diseases as disclosed hereinafter, such as obesity, body overweight condition and comorbidities thereof. Said pharmaceutical compositions, which are suited for administration to a human being in the need thereof, typically comprise a therapeutically effective amount of the compound of the invention.

The pharmaceutical composition of the invention may be in a solid or liquid dosage form. In a preferred aspect of the invention, the dosage form is a liquid injectable form.

The pharmaceutical composition of the invention may be in unit dosage form, containing a mono-dose or more doses. In a preferred aspect of the invention, the unit dosage form may be provided in injectable form in a device for application (e.g. pre-filled ready-to-use injection device), wherein the device for application can be a syringe, a pen injector or an auto-injector containing the liquid pharmaceutical composition.

Subcutaneous administration is the most common route of application of therapeutic peptides generally, and also seems suitable for the compounds of general formula (I), or the pharmaceutical compositions thereof. For subcutaneous administration a single-use device (provides the quantity of the compound for a single dosage unit) or a multiple-use device (provides the quantity of the compound for more than one dosage unit) can be used. Suitable drug-delivery devices for subcutaneous administration comprise auto-injectors (e.g. for single dosage unit) or pen injectors (e.g. for more than one dosage unit) containing a prefilled syringe or a cartridge with a liquid pharmaceutical composition of the compound of the invention, such as an aqueous formulation.

The invention also refers to a drug-delivery device for subcutaneous administration comprising at least a compound of general formula (I) or a pharmaceutical composition thereof (pharmaceutically acceptable salts included), preferably wherein the device is an auto-injector or a pen injector.

### Method of Treatment/Use in Treatment

The present invention relates to NMU-8 analogues of general formula (I) or pharmaceutical compositions comprising at least one compound of the invention, including pharmacological acceptable salts thereof, which are suitable for use in the prevention or treatment of a disease or condition associated with or modulated by NMU receptor 2 (NMUR2) activation.

Accordingly, the present invention relates to NMU-8 analogues of general formula (I) or pharmaceutical compositions comprising at least one compound of the invention, including pharmacological acceptable salts thereof, for use as a medicament.

The invention further provides NMU-8 analogues according to general formula (I) or pharmaceutical compositions thereof, including the pharmacological acceptable salts thereof, for use in a method of prevention and/or treatment of conditions and diseases mentioned hereinafter.

The compounds of the present invention are suitable for use in the reduction of food intake, increase energy expenditure, promotion of body-weight loss (weight reduction), and/or inhibition, reduction or prevention of body-weight gain, or chronic weight management.

By “prevention of body-weight gain” is meant inhibiting or reducing when compared to the absence of treatment and is not necessarily meant to imply complete cessation of body-weight gain.

Furthermore, the compounds of the invention may be used for direct or indirect therapy of any condition caused or characterised by body overweight (e.g. body-mass-index (BMI) greater than 25), preferably for the treatment or prevention of obesity (e.g. BMI greater than 30), morbid obesity (e.g. BMI greater than 35 with at least one weight-related comorbidity or BMI greater than 40), obesity-linked inflammation, obesity-linked gallbladder disease, and/or obesity related obstructive sleep apnea.

It will be understood that the NMU-8 analogues of the present invention may thus be administered to subjects affected by conditions characterised by inadequate control of appetite or otherwise over-feeding, such as binge-eating disorder and Prader-Willi syndrome.

The compounds of the invention may also be suitable for use in the prevention or treatment of diseases or conditions different from body overweight control and obesity and selected in the list comprising: glucose metabolism diseases including: type 2 diabetes (T2D), impaired fasting glucose (IFG), and impaired glucose tolerance (IGT);
metabolic diseases including: metabolic syndrome, hypertension, dyslipidemia, atherogenic dyslipidemia; kidney and liver diseases including: chronic kidney diseases (CKD), hepatic steatosis, non-alcoholic fatty liver disease (NAFLD), and non-alcoholic steatohepatitis (NASH);
cardiovascular diseases including: macrovascular disease, atherosclerosis, coronary heart disease, peripheral artery disease, stroke and microvascular disease;
degeneration of cartilage, and osteoarthritis;
reproductive health complications of obesity or body overweight, such as infertility; cancer; and/or
neuropsychiatric conditions including: cognitive impairment, anxiety and major depressive disorders.

Effects of NMU-8 analogues of the present invention on the above-mentioned diseases or conditions different from body overweight control and obesity (i.e. T2D, IGT, CKD, NAFLD, NASH, etc.) may be a result of or associated with the effect of the compounds of the invention on body overweight/obesity or may be independent thereof.

Therefore, the compounds of the invention may be suitable for use in the prevention or treatment of body over-weight associated diseases or obesity associated disease (weight-related comorbidities) selected in the list comprising: glucose metabolism diseases such as type 2 diabetes (T2D), impaired fasting glucose (IFG), and impaired glucose tolerance (IGT); metabolic diseases such as metabolic syndrome, hypertension, dyslipidemia, and atherogenic dyslipidemia; kidney and liver diseases such as chronic kidney diseases (CKD), hepatic steatosis, non-alcoholic fatty liver disease (NAFLD), and non-alcoholic steatohepatitis (NASH); cardiovascular diseases such as macrovascular disease, atherosclerosis, coronary heart disease, peripheral artery disease, stroke and microvascular disease; degeneration of cartilage, and osteoarthritis; reproductive health complications of obesity or body overweight, such as infertility; cancer; and neuropsychiatric conditions such as cognitive impairment, anxiety, and major depressive disorders.

The compounds of the invention may also be used for lowering circulating LDL cholesterol levels and/or increasing HDL/LDL ratio (LDL: low-density lipoprotein, HDL: high-density lipoprotein).

It has been reported that an adequate loss of body weight is expected to improve obesity-associated comorbidities such as liver histology and function found in NASH patients (Promrat et al., Hepatology. 2010; 51: 121-129; Al-Jiffri et al., Afr Health Sci. 2013; 13: 667-672; Tilg, Moschen, Minerva Gastroenterol Dietol. 2010; 56(2):159-167).

The subject treated with a compound of the present invention may be affected by body overweight or obesity accompanied by at least one weight-related comorbidity mentioned hereinbefore, preferably type 2 diabetes, a metabolic disease such as hypertension or dyslipidemia, a liver disease such as NASH, or a cardiovascular disease.

It will be clear that the NMU-8 analogues of the present invention can be used for treatment of combinations of diseases, disorders or conditions hereinbefore described.

In a further aspect the present invention relates to the use of NMU-8 analogues according to general formula (I) or pharmaceutic compositions comprising at least one compound of the invention, including the pharmacological acceptable salts thereof, for the preparation of a medicament for the treatment and/or prevention of diseases or conditions mentioned hereinbefore.

In a further aspect the present invention relates to methods for the treatment or prevention of diseases and conditions mentioned hereinbefore, which method comprises the administration of a therapeutically effective amount of an NMU-8 analogue according to general formula (I) or pharmaceutic compositions thereof, including the pharmaceutically acceptable salts thereof, to a human being in the need thereof.

The actual therapeutically effective amount or therapeutic dosage usually depend on factors known by those skilled in the art such as age and weight of the patient, route of administration and severity of disease. In any case, the compounds are administered at dosages and in a manner, which allows a pharmaceutically effective amount to be delivered based upon patient's unique condition.

Compounds of the invention may be administered subcutaneously to a human being in the need thereof at a dose range from 0.1 mg to 100 mg.

Compounds of the invention may be administered subcutaneously to a human being in the need thereof by once-weekly dosing, every other week dosing or once monthly dosing, depending on the pharmacokinetic profile of the compound (i.e. MRT).

### Combination Therapy

NMU-8 analogues according to the present invention may be administered as part of a combination therapy together with at least one further active agent to a subject in the need thereof, for use in a method of prevention and/or treatment of one or more conditions or diseases which are in the focus of the present invention. Preferably, said further active agent is an agent known in the art for the treatment of obesity, diabetes, dyslipidemia or hypertension, more preferably an anti-obesity agent.

The compound of the invention and said at least one further active agent may be administered as active ingredients combined in the same pharmaceutical composition or formulation (fixed-dose combination, briefly FDC) or, alternatively, as active ingredients of separate pharmaceutical compositions or formulations (free dose combination). In a free dose combination, the at least two pharmaceutical compositions can be administered simultaneously or sequentially.

In a free dose combination, the NMU-8 analogue of the present invention may be for use as adjunctive therapy (add-on therapy) to a drug used on the market for the treatment of one or more conditions or diseases mentioned hereinbefore and hereinafter, preferably a drug used for the treatment of obesity or comorbidities thereof.

The administration of an NMU-8 analogue of the present invention in combination with at least one further active agent, such as a current anorexigenic peptide, leads to a medicament for the prevention or treatment of conditions or diseases mentioned hereinbefore and hereinafter, preferably body overweight condition, obesity or comorbidities thereof, that provides an additive or synergistic therapeutic effect (e.g. an anorexigenic effect) in comparison to the effects provided by the individual active agents in said therapy.

In one aspect, the present invention is direct to an NMU-8 analogue of the present invention and at least one further active agent formulated together in a pharmaceutical composition (FDC) and use thereof in a method of prevention and/or treatment of conditions or diseases mentioned hereinbefore and hereinafter, such as body overweight condition, obesity and/or comorbidities thereof. Preferably, said further active agent is an agent for use in the treatment of obesity, diabetes, dyslipidemia or hypertension, more preferably an anti-obesity agent.

In one aspect, the present invention relates to a pharmaceutical composition comprising an NMU-8 analogue of the present invention and at least one further active agent and use thereof in a method of prevention and/or treatment of conditions or diseases mentioned hereinbefore and hereinafter, such as body overweight condition, obesity or comorbidities thereof. Preferably, said further active agent is an agent for use in the treatment of obesity (i.e. anti-obesity agent), diabetes, dyslipidemia or hypertension, more preferably an anti-obesity agent.

According to a preferred aspect, an NMU-8 analogue of the present invention may be administered in combination with at least one further anti-obesity agent, in a fixed dose combination (FDC) or a free-dose combination. In case of free dose combination, said at least one further anti-obesity agent may be formulated for subcutaneous administration or oral administration.

Said further anti-obesity agent may be a glucose-dependent insulinotropic polypeptide (GIP) receptor agonist (including GIP or a GIP analogue, LY-3537021, LY-3532226), or a glucagon like peptide-1 (GLP-1) receptor agonist (including GLP-1 or a GLP-1 analogue, exendin-4 or an exendin-4 analogue, liraglutide (Saxenda™ or Victoza™), semaglutide (Wegovy™ or an oral formulation), dulaglutide, albiglutide, MK-8521, danuglipron (PF-068829614 oral formulation), PF-07081532 (oral formulation), LY-3502970, XW-003, XW014), or a glucagon receptor (GCGR) agonist (including glucagon or a glucagon analogue, HM-15136), or oxyntomodulin or an oxyntomodulin analogue (e.g. TT-401), or a GCGR/GLP-1R dual agonist (e.g. HM-12525, SAR-425899, MEDI-0382, NN-9277, marzdutide (LY-3305677), pemvidutide (ALT-801), DD-01) or a GLP-1R/GIPR dual agonist (e.g. tirzepatide, LY-3493269, AMG-133, CT-388, CT-868, SCO-094, semaglutide/NN-9389), or a GLP-1R/GIPR/GCGR triple agonist (e.g. NN-9423, LY-3437943).

Said further anti-obesity agent may be a peptide YY (PYY) or a PYY analogue (e.g. NN-9775, LY-3457263, Y-14), or a neuropeptide Y (NPY) or a NPY analogue, or a NPY1R antagonist or inverse agonist, or a NPY2R agonist or a NPY4R agonist, or a NPY5R antagonist; or may be a GLP-1R/NPY receptor dual agonist, including a combination of a GLP-1 analogue and a PYY analogue (preferably a NPY2R agonist) such as a combination of semaglutide/PYY analogue (e.g. semaglutide/NN-9775) or dulaglutide/PYY analogue (e.g. dulaglutide/LY-3457263); or may be a GLP-1R/GIPR/NPY receptor triple agonist, such as a combination of a GLP-1R/GIPR dual agonist and a PYY analogue or a NPY receptor agonist, preferably a NPY2R agonist (e.g. tirzepatide/LY-3457263); or may be a GLP-1R/GCGR/NPYR triple agonist, such as a combination of a GLP-1R/GCGR dual agonist and a PYY analogue or a NPYR agonist, preferably a NPY2R agonist (e.g. marzdutide/PYY analogue, pemvidutide/PYY analogue).

Said further anti-obesity agent may be an amylin receptor agonist, including amylin or an amylin analogue such as pramlintide, NN-9838, cagrilintide (AM-833); or may be a GLP-1R/amylin receptor dual agonist, such as a combination of semaglutide/cagrilintide (i.e. cagrisema), or amycretin (oral formulation).

Alternatively, said further anti-obesity agent may be a further NMUR2 agonist, orlistat, sibutramine, phentermine, a melanin concentrating hormone receptor 1 antagonist, cholecystokinin (CCK), a leptin analogue, a ghrelin O-acyltransferase (GOAT) inhibitor, a ghrelin-receptor antagonist, a cannabinoid receptor 1 antagonist, a beta-3 agonist, a lipase inhibitor, human proislet peptide (HIP), a melanocortin receptor 4 agonist, a adiponectin receptor agonist, a G-protein-coupled receptor 40 (GPR40; also known as free fatty acid receptor 1, FFAR1) agonist, a mitochondrial modulator, a thyroid hormone receptor agonist, a growth differentiation factor 15 (GDF15) agonist (e.g. AMG-171, JNJ-9090, NNC0247-0829), or a enteropeptidase inhibitor, as well as analogues thereof.

According to a preferred aspect, an NMU-8 analogue of general formula (I) or pharmaceutically acceptable salts thereof, preferably a compound of table 3, more preferably a compound of table 6, may be administered as part of a combination therapy (free-dose combination) together with a further anti-obesity agent selected from the list consisting of semaglutide (subcutaneous formulation or oral formulation), danuglipron, PF-07081532 (GLP-1Ra oral formulation), tirzepatide, pemvidutide, LY-3437943 (GLP-1R/GIPR/GCGR triple agonist), cagrisema, amycretin (oral formulation), and combination semaglutide/NN-9775 (PYY analogue), for use in a method of prevention or treatment of obesity, body overweight condition and/or comorbidities thereof as disclosed hereinbefore.

According to a more preferred aspect, an NMU-8 analogue of general formula (I) or pharmaceutically acceptable salts thereof, preferably a compound of table 3, more preferably a compound of table 6, may be administered as part of a combination therapy (free-dose combination) together with semaglutide, wherein semaglutide is formulated for subcutaneous administration (Wegovy™) or for oral administration, for use in a method of prevention or treatment of obesity, body overweight condition and/or comorbidities thereof as disclosed hereinbefore (for example, semaglutide and compound No. 1 of table 3, semaglutide and compound No. 7 of table 3, semaglutide and compound No. 26 of table 3, semaglutide and compound No. 33 of table 3, semaglutide and compound No. 42 of table 3, semaglutide and compound No. 43 of table 3, or semaglutide and compound No. 69 of table 3).

According to a preferred aspect, an NMU-8 analogue of general formula (I), preferably an NMU-8 analogue of table 3, more preferably an NMU-8 analogue of table 6, or pharmaceutically acceptable salts thereof, may be administered as part of a combination therapy (free dose combination or FDC) with the GCGR/GLP-1R dual agonist of formula (III)

(III)
(SED ID NOS 87-88)
H-H-Ac4c-QGTFTSDYSKYLDERAAKDFI-K([17-carboxy-
heptadecanoyl]-gGlu-GSGSGG-)-WLESA-NH₂,

or pharmaceutically acceptable salts thereof, for use in a method of prevention or treatment of obesity, body overweight condition and/or comorbidities thereof as disclosed hereinbefore (for example, compound of formula (III) and compound No. 1 of table 3, compound of formula (III) and compound No. 7 of table 3, compound of formula (III) and compound No. 26 of table 3, compound of formula (III) and compound No. 33 of table 3, compound of formula (III) and compound No. 42 of table 3, compound of formula (III) and compound No. 43 of table 3, or compound of formula (III) and compound No. 69 of table 3).

In a fixed-dose combination (FDC), an NMU-8 analogue of general formula (I), preferably a compound of table 3, more preferably a compound of table 6, may be formulated in a pharmaceutical composition together with the GCGR/GLP-1R dual agonist of formula (III), including pharmaceutically acceptable salts thereof, and optionally together with at least one pharmaceutically acceptable adjuvant, diluent and/or carrier. Preferably the pharmaceutical composition is suitable for subcutaneous administration to a subject in need of therapy.

Furthermore, the present disclosure provides a pharmaceutical composition comprising a NMU-8 analogue of general formula (I), preferably a compound of table 3, more preferably a compound of table 6, the GCGR/GLP-1R dual agonist of formula (III), including pharmaceutically acceptable salts thereof, and optionally at least one pharmaceutically acceptable adjuvant, diluent and/or carrier.

The GCGR/GLP-1R dual agonist of formula (III) is compound No 13 of paragraph [0164] of the patent EP3057984 B1, herewith incorporated by reference. It will be understood from the skilled in the art that the term [17-carboxy-heptadecanoyl]-gGlu-GSGSGG- (SEQ ID NO: 88) is synonym of the terms [17-carboxy-heptadecanoyl]-isoGlu-GSGSGG- (SEQ ID NO: 88) and HOOC—(CH₂)₁₆—(CO)-isoGlu-GSGSGG- (SEQ ID NO: 88) used in EP3057984 B1. The structure of the substituent [17-carboxy-heptadecanoyl]-gGlu-GSGSGG- (SEQ ID NO: 88) (i.e. [17-carboxy-heptadecanoyl]-isoGlu-GSGSGG- (SEQ ID NO: 88)) is disclosed in paragraph [0099]-(vii) of EP3057984, herewith incorporated by reference. In the compound of formula (III) the acyl group of the terminal residue G of the side chain [17-carboxy-heptadecanoyl]-gGlu-GSGSGG- (SEQ ID NO: 88) is linked via an amide bond to the amino group of the K residue at position 24 of the backbone chain.

In the compound of formula (III) the Ac4c residue denotes 1-amino-cyclobutanecarboxylic acid.

In the compound of formula (III) the first H denotes a hydrogen, and the second H denotes a histidine residue.

Compound of formula (III) is glucagon receptor and GLP-1R dual agonist as determined by his capability to stimulate intracellular cAMP formation in appropriate assays, as disclosed in EP3057984 B1 at Example 2—Table 1 at page 21 (compound No 13), and Examples 3 to 4—Table 2 to 3 at pages 22-23 (compound No 13), herewith incorporate by reference. The dual agonist may combine the effect of glucagon receptor, e.g. on fat metabolism, with the effect of GLP-1R, e.g. on blood glucose levels and food intake. Compound of formula (III), may therefore act to accelerate elimination of excessive adipose tissue, induce sustainable weight loss, and improve glycaemic control. Thus, it may be used in a subject in need thereof as therapeutic agents for preventing weight gain, promoting weight loss, reducing excess body weight, treating body overweight condition or treating obesity, as well as associated diseases and health conditions (e.g. as disclosed in EP3057984 B1 paragraphs [0135] to [0145], herewith incorporate by reference).

According to a further aspect of the present invention, an NMU-8 analogue of the present invention may be administered in addition to a diet and/or physical exercises, for use in a method of prevention and/or treatment of one or more conditions or diseases mentioned hereinbefore, preferably body overweight condition, obesity and/or comorbidities thereof, in a subject in the need thereof.

Moreover, an NMU-8 analogue of the invention according to general formula (I), preferably a compound of table 3, more preferably a compound of table 6, may be administered in combination with an anti-diabetic agent of known type, including metformin, a sulfonylurea (e.g. glibenclamide, tolbutamide, glimepiride, glipizide, gliquidon, glibornuride, gliclazide or analogue thereof), a alpha-glucosidase blockers (e.g. acarbose, voglibose, miglitol or analogue thereof), a glinide (e.g. nateglinide, repaglinide or analogue thereof), a glitazone (e.g. pioglitazone, rosiglitazone or analogue thereof), a DPP-IV inhibitor (e.g. sitagliptin, saxagliptin, linagliptin, alogliptin or analogue thereof), a SGLT2 inhibitor (e.g. a gliflozin such as empagliflozin, canagliflozin, dapagliflozin, ipragliflozin, sotagliflozin, ertugliflozin or analogue thereof) a GLP-1R agonist (e.g. tirzepatide, semaglutide, liraglutide, exenatide (Byetta™, Bydureon™, Byetta LAR™), or lixisenatide), a GPR40 agonist (FFAR1/FFA1 agonist, e.g. fasiglifam), or an insulin or an insulin analogue (e.g. insulin glargin, insulin aspart, insulin lispro, human insulin (rDNA), insulin detemir, insulin degludecand, or insulin glulisine), for use in a method of prevention and/or treatment of one or more conditions or diseases mentioned hereinbefore.

An NMU-8 analogue of the present invention may be administered in combination with an anti-dyslipidemia agent of known type, including, but not limited to, a statin, a fibrate, a niacin, a PSCK9 (proprotein convertase subtilisin/kexin type 9) inhibitor, or a cholesterol absorption inhibitor, for use in a method of prevention and/or treatment of one or more conditions or diseases mentioned hereinbefore.

An NMU-8 analogue of the present invention may be administered in combination with medications targeting cardiovascular diseases treating hypertension, dyslipidemia, inflammation, and platelet function for use in a method of prevention and/or treatment of one or more conditions or diseases mentioned hereinbefore. The medication treating hypertension can be selected from an angiotensin-converting enzyme inhibitor, an angiotensin II receptor blocker, a diuretic, a beta-blocker and a calcium channel blocker.

In a further aspect, the present disclosure provides a kit comprising an NMU-8 analogue of general formula (I) or a pharmaceutically acceptable salt thereof, an optional pharmaceutically acceptable carrier, and a package insert comprising instructions for administration to a subject in the need of treating a disease, disorder or condition associated with or modulated by NMUR2 activation, such as obesity, body over-weight and comorbidities thereof.

In a further aspect, the present disclosure provides a kit comprising an NMU-8 analogue of general formula (I) or a pharmaceutically acceptable salt thereof, at least one further active agent for the treatment of obesity, diabetes, dyslipidemia or hypertension, preferably a further active agent for the treatment of obesity mentioned hereinbefore, and a package insert comprising instructions for administration to a subject in the need of treating a disease, disorder or condition disclosed hereinbefore.

### Biological Assays and Data

### EXAMPLES

The following examples demonstrate certain specific embodiments of the present invention. The following examples were carried out using standard techniques that are well known and routine to those of skill in the art, except where otherwise described in detail.

### Example 1: Radioligand Binding Competition Assay (RLB) Human NMUR2 Receptor Binding

### 1) Membranes

The test is based on membranes prepared from a Human NMUR2/Apoaequorin Stable Cell Line (Creative Biogene, Cat. No. CSC-RG1309).

Cell Line Description: CHO-NMUR2 cell line is clonally derived from a CHO-K1 cell line, which has been transfected with a bicistronic expression plasmid containing the sequence coding for the human neuromedin U receptor 2 (AF242874.1). MOSAIC-ID: CL16550616.

Membrane preparation: Cells were broken by nitrogen cavitation (900 psi, 1 hour). Intact cells were removed by centrifugation (400 rcf (relative centrifugal force), 10 minutes, 4° C.) before the crude membrane fraction was collected by ultracentrifugation (20.000 rcf, 1 hour, 4° C.).

### 2) Human NMUR2 Receptor Binding Assay (Radioligand: [125I]-Neuromedin U-25 (Human))

In this Scintillation Proximity Assay (SPA) design, the membranes immobilize directly to the PVT-WGA-PEI type A beads (polyethyleneimine treated polyvinyltoluene beads coupled to wheatgerm agglutinin, Perkin Elmer) via interactions of wheat germ agglutinate and glycosylated membrane structures. Thereby the radioactively la-belled ligand, bound to NMUR2 receptors on the membrane, comes in close proximity to the solid scintillator of the PVT bead. Radioactive human neuromedin U-25 then stimulates the scintillant to emit light. In contrast, radioligand not bound to the receptor is too distant from the bead to elicit a signal.

For the Scintillation Proximity Assay the CHO-K1 hNMUR2 cell membranes and SPA-beads are diluted to concentrations, which assure a robust signal to background ratio. The SPA incubation buffer consists of 25 mM Hepes (pH=7.0), 5 mM MgCl₂, 1 mM CaCl₂, 10 ug/mL Saponine and 0.1% bovine serum albumin and is used for all dilutions.

Final assay setup in 96-well plate: 20 μL of 10× test compound dilutions+40 μL of 5× [125l]-Neuromedin U-25 (1× approx. 30 pM; 2000 μCi/mmol)+60 μL membrane dilution (approx. 2.5 μg membrane protein)+80 μL SPA bead dilution (0.75 mg).

The assay is incubated for 4 hours at RT, including controls for specific binding where no displacer is added and non-specific binding by adding unlabeled hNMU-25 at 100 nM. Bound radioactivity is determined by scintillation counting (MicroBeta Trilux 1450, Perkin Elmer, detection of 125I decay). The test compound affinity (Ki) is calculated as described below for which the dissociation constant (Kd) and the exact concentration of the radioligand in the assay is given. The system assumes one binding site with no relevant radioligand depletion (<10%) and that the binding is reversible and at equilibrium.

$$Ki = xC 50 / ( ( Ligand / Kd ) + 1 )$$

$$Y = Bottom + ( Top - Bottom ) / 1 + 10 \hat{} ( ( Log IC 50 - X ) * Slope ) )$$

### Example 2: Radioligand Binding Competition Assay (RLB) Human NMUR1 Receptor Binding

### 1) Membranes

The test is based on membranes prepared from a human NMUR1/Apoaequorin Stable Cell Line (Creative Biogene, Cat. No. CSC-RG1308).

Cell Line Description: CHO-NMUR1 cell line is clonally-derived from a CHO-K1 cell line, which has been transfected with a bicistronic expression plasmid containing the sequence coding for the human neuromedin U receptor 1 (NM_006056.4, 180 to 1391). Mosaic-ID: CL16550614

Membrane preparation: Cells were broken by nitrogen cavitation (900 psi, 1 hour). Intact cells were removed by centrifugation (400 rcf, 10 minutes, 4° C.) before the crude membrane fraction was collected by ultracentrifugation (20.000 rcf, 1 hour, 4° C.).

2) Human NMUR1 Receptor Binding Assay (Radioligand: [125l]-Neuromedin U-25)

In this Scintillation Proximity Assay (SPA) design, the membranes immobilize directly to the PVT-WGA-PEI type A beads (polyethyleneimine treated polyvinyltoluene beads coupled to wheatgerm agglutinin, Perkin Elmer) via interactions of wheat germ agglutinate and glycosylated membrane structures. Thereby the radioactively la-belled ligand, bound to NMUR1 receptors on the membrane, comes in close proximity to the solid scintillator of the PVT bead. Radioactive human neuromedin U-25 then stimulates the scintillant to emit light. In contrast, radioligand not bound to the receptor is too distant from the bead to elicit a signal.

For the Scintillation Proximity Assay the CHO-K1 hNMUR1 cell membranes and SPA-beads are diluted to concentrations, which assure a robust signal to background ratio. The SPA incubation buffer consists of 25 mM Hepes (pH=7.0), 5 mM MgCl₂, 1 mM CaCl₂, 10 ug/mL Saponine and 0.1% bovine serum albumin and is used for all dilutions.

Final assay setup in 96-well plate: 20 μL of 10× test compound dilutions+40 μL of 5× [125l]-Neuromedin U-25 (1× approx. 30 pM; 2000 μCi/mmol (μCi=microcurie))+60 μL membrane dilution (approx. 1.0 μg membrane protein)+80 μL SPA bead dilution (250 μg).

The assay is incubated for 4 hours at RT, including controls for specific binding where no displacer is added and non-specific binding by adding unlabeled hNMU-25 at 100 nM. Bound radioactivity is determined by scintillation counting (MicroBeta Trilux 1450, Perkin Elmer, detection of 125I decay). The test compound affinity (Ki) is calculated as described below, for which the dissociation constant (Kd) and the exact concentration of the radioligand in the assay is given. The system assumes one binding site with no relevant radioligand depletion (<10%) and that the binding is reversible and at equilibrium.

$$Ki = xC 50 / ( ( Ligand / Kd ) + 1 )$$

$$Y = Bottom + ( Top - Bottom ) / 1 + 10 \hat{} ( ( Log IC 50 - X ) * Slope ) )$$

The results for the RLB assays are summarized in Table 4, below:

| | Ki | Ki |
|---|---|---|
| Compound | (hNMUR1) | (hNMUR2) |
| No. | [nM] | [nM] |
| 1 | >10000 | 8.6 |
| 6 | >10000 | 10.1 |
| 7 | 3744 | 22.8 |
| 8 | >10000 | 89.1 |
| 9 | >10000 | 58.3 |
| 10 | >10000 | 204.3 |
| 11 | >10000 | 76.3 |
| 12 | >10000 | 36.2 |
| 20 | >10000 | 23.7 |
| 21 | >10000 | 55.9 |
| 22 | >10000 | 45.8 |
| 23 | >10000 | 38.8 |
| 24 | >10000 | 52.3 |
| 25 | >10000 | 23.1 |
| 26 | >10000 | 48.1 |
| 27 | >10000 | 23.4 |
| 28 | >10000 | 56.2 |
| 33 | >10000 | 38.5 |
| 38 | >10000 | 33.8 |
| 42 | >10000 | 23.7 |
| 43 | >10000 | 32.6 |
| 65 | 722.4 | 25.3 |
| 69 | >10000 | 25.4 |

### Example 3: IP1 Gq HTRF Assay Activation of Human NMUR2 or Human NMUR1 in Buffer

Application of the Homogenous Time Resolved Fluorescence (HTRF) technology optimized for Gq coupled receptors has been described in detail in the Cisbio IP-One Gq kit manual. In brief, a FRET signal (FRET: fluorescence resonance energy transfer) is generated between the donor label of an IP-One antibody (anti-IP1-Tb) and the acceptor label of a tagged IP-One (IP1-d2). Agonism of a Gq coupled receptor increases intracellular IP-One, which competes with labeled IP-One and leads to a reduction of the FRET signal.

Frozen stocks of CHO-K1 cells stably expressing the human NMUR1 or NMUR2 receptor were thawed and 5000 cells were seeded in culture medium (HAM'S F12, 10% FCS) into each well of a 384-well assay plate. After seeding, cells were incubated for 24 h in a humidified incubator at 37° C. and 5% CO₂. Next, cells were washed once with 50 μL of wash buffer (20 mM Hepes, 1×HBSS, pH 7.4), followed by addition of either 10 μL of stimulation buffer (20 mM Hepes, 1×HBSS, 50 mM LiCl, 0.1% BSA, pH 7.4). Peptide agonists to be tested were dissolved in 100% DMSO and pre-diluted in stimulation buffer at a suitable concentration range for an 8 pt dose-response curve. 5 μL of peptide pre-dilution were then added to each well, followed by another 5 μL of stimulation buffer to give a final concentration of 1% DMSO. Samples were subsequently incubated for 1 hour in a humidified incubator at 37° C. After addition of 5 μL each of IP1-d2 and anti-IP1-Tb, samples were incubated for another 1 hour at room temperature and FRET signals (ratio 665 nm/620 nm) were measured. EC₅₀ values were calculated by nonlinear regression using a 4-parameter sigmoidal curve fit.

### Example 4: IP1 Gq HTRF Assay Activation of Human NMUR2 in Human Plasma

Application of the Homogenous Time Resolved Fluorescence (HTRF) technology optimized for Gq coupled receptors has been described in detail in the Cisbio IP-One Gq kit manual. In brief, a FRET signal is generated between the donor label of an IP-One antibody (anti-IP1-Tb) and the acceptor label of a tagged IP-One (IP1-d2). Agonism of a Gq coupled receptor increases intracellular IP-One, which competes with labeled IP-One and leads to a reduction of the FRET signal.

Frozen stocks of CHO-K1 cells stably expressing the human NMUR1 receptor or human NMUR2 receptor were thawed and 5000 cells were seeded in culture medium (HAM'S F12, 10% FCS) into each well of a 384-well assay plate. After seeding, cells were incubated for 24 h in a humidified incubator at 37° C. and 5% CO₂. Next, cells were washed once with 50 NL of wash buffer (20 mM Hepes, 1×HBSS, pH 7.4), followed by addition of μL of plasma (100% human plasma, 50 mM LiCl). Peptide agonists to be tested were dissolved in 100% DMSO and pre-diluted in plasma at a suitable concentration range for an 8 pt dose-response curve. 5 μL of peptide pre-dilution were then added to each well, followed by another 5 μL of plasma to give a final concentration of 1% DMSO. Samples were subsequently incubated for 1 hour in a humidified incubator at 37° C. The plasma solution was replaced by 20 μL of stimulation buffer. After addition of 5 μL each of IP1-d2 and anti-IP1-Tb, samples were incubated for another 1 hour at room temperature and FRET signals (ratio 665 nm/620 nm) were measured. EC₅₀ values were calculated by nonlinear regression using a 4-parameter sigmoidal curve fit.

The results for the IP1 Gq HTRF assays are summarized in Table 5, below:

| | EC50 | EC50 | EC50 |
|---|---|---|---|
| | (hNMUR1) | (hNMUR2) | (hNMUR2) |
| Compound | buffer | buffer | human |
| No. | [nM] | [nM] | plasma [nM] |
| 1 | 890 | 1.4 | 2.8 |
| 2 | 220 | 1.1 | 3.7 |
| 3 | 440 | 1.9 | 5.6 |
| 4 | 770 | 2 | 5.8 |
| 5 | 870 | 1.8 | 10.0 |
| 6 | 120 | 2.0 | 6.9 |
| 7 | 250 | 1.2 | 4.4 |
| 8 | 1000 | 3.1 | 9.0 |
| 9 | 220 | 2.8 | 5.8 |
| 10 | 720 | 4.1 | 9.8 |
| 11 | 1100 | 2.8 | 10.6 |
| 12 | 1000 | 2.7 | 12.9 |
| 13 | 1200 | 3.5 | 10.2 |
| 14 | 1400 | 2.9 | 10.3 |
| 15 | 860 | 3.2 | 10.8 |
| 16 | >1000 | 3.3 | 11.0 |
| 17 | 610 | 4.7 | 24.8 |
| 18 | >1000 | 7.0 | 34.7 |
| 19 | 1400 | 5.7 | 36.6 |
| 20 | >1000 | 2.2 | 12.1 |
| 21 | >1000 | 2.6 | 8.7 |
| 22 | >1000 | 3.2 | 10.8 |
| 23 | >1000 | 3.3 | 11.1 |
| 24 | 1000 | 1.4 | 12.6 |
| 25 | 200 | 1.1 | 8.6 |
| 26 | >1000 | 1.5 | 3.9 |
| 27 | 1700 | 1.2 | 6.9 |
| 28 | >1000 | 1.4 | 10.8 |
| 29 | >1000 | 52.0 | 535 |
| 30 | >1000 | 67.0 | 725 |
| 31 | >1000 | 3.7 | 14.6 |
| 32 | 1200 | 6.5 | 31.2 |
| 33 | 770 | 1.8 | 9.5 |
| 34 | >1000 | 9.0 | 24.6 |
| 35 | 190 | 1.5 | 9.4 |
| 36 | 1400 | 4.7 | 18.3 |
| 37 | 1300 | 3.5 | 27.1 |
| 38 | >1000 | 1.9 | 9.7 |
| 39 | 190 | 2.5 | 10.4 |
| 40 | 950 | 4.5 | 11.3 |
| 41 | 1300 | 2.0 | 12.2 |
| 42 | 1500 | 3.2 | 5.8 |
| 43 | >1000 | 3.9 | 10.1 |
| 44 | >1000 | 2.2 | 20.5 |
| 45 | >1000 | 30.0 | 129 |
| 46 | >1000 | 13.0 | 26.2 |
| 47 | >1000 | 9.3 | 32.6 |
| 48 | >1000 | >1000 | >1000 |
| 49 | 1300 | 11.0 | 25.2 |
| 50 | >1000 | 23.0 | 69.9 |
| 51 | >1000 | 19.0 | 112 |
| 52 | 200 | 1.4 | 6.4 |
| 53 | 1300 | 4.0 | 24.9 |
| 54 | >1000 | 28.0 | 122 |
| 55 | >1000 | 29.0 | 294 |
| 56 | >1000 | 72.0 | 432 |
| 57 | >1000 | 110 | 509 |
| 58 | >1000 | 140 | 709 |
| 59 | >1000 | 5.0 | 12.1 |
| 60 | 540 | 4.0 | 13.9 |
| 61 | >1000 | 4.6 | 14.1 |
| 62 | 440 | 3.1 | 14.2 |
| 63 | 670 | 4.5 | 14.9 |
| 65 | 130 | 2.8 | 11.7 |
| 66 | 550 | 1.6 | 18.9 |
| 67 | 90 | 0.9 | 9.9 |
| 68 | 82 | 1.2 | 9.6 |
| 69 | 660 | 1.3 | 8.7 |
| 70 | >1000 | 2.5 | 27.2 |
| 71 | 130 | 1.5 | 14.5 |
| 72 | 150 | 0.9 | 10.4 |
| 73 | 540 | 1.9 | 15.6 |
| 74 | 1400 | 11.0 | 72.8 |
| 75 | 130 | 0.7 | 3.5 |
| 76 | 1100 | 1.1 | 6.2 |
| 77 | 110 | 0.8 | 5.2 |
| 78 | 72 | 1.2 | 5.3 |
| 79 | >1000 | 1.3 | 7.0 |
| 80 | 470 | 1.9 | 7.1 |

### Example 5: Pharmacokinetics Intravenous (iv) PK in Mouse

Pharmacokinetic parameters of the test compounds were determined after intravenous administration to Naval Medical Research Institute (NMRI) mice. Male NMRI mice were obtained either from Charles River (Germany) or from Janvier (France) weighing approximately 30 g to 40 g. Mice were housed in standard cages with light cycle of 12-hour dark and 12-hour light. Standardized food and water were offered ad libitum to the animals during the whole experimental period. The respective peptide was dissolved in 50 mM phosphate buffer (pH 7.4) containing 3.5% mannitol. Intravenous doses of 30 nmol/kg were given via a tail vein. Serial blood samples were collected from the vena saphena into tubes containing EDTA as anticoagulant at different time points up to 48 hours post dosing. After centrifugation for approximately 5 minutes, plasma samples were transferred to 96-well Polymerase Chain Reaction (PCR) plates, immediately frozen and kept at approximately −20° C. until analyzed for plasma concentration by liquid chromatography mass spectrometry (LC-MS/MS). Individual plasma concentration-time profiles were analyzed by a non-compartmental approach, and the resulting pharmacokinetic parameters were determined.

The results for mouse PK are summarized in Table 6, below:

| | Mean residence |
|---|---|
| | time (MRT) |
| Compound | after iv- |
| No. | application [h] |
| 1 | 38.1 |
| 7 | 17.4 |
| 26 | 12.4 |
| 33 | 18.7 |
| 42 | 31.2 |
| 43 | 37.7 |
| 69 | 20.2 |

### Example 6: Solubility Determination

Peptides (as TFA-salts) were weighed out in a filter unit (Mini-UniPrep Syringeless Filter 0.45 μm, Whatman PVDF), and 0.1 M phosphate buffer at pH 6 to 8 was added to achieve 10 mg/mL final peptide concentration. The peptides were dissolved by shaking the filter units horizontally at 350 rpm for 2 hours at room temperature. Samples were then filtered to remove any insoluble particles. Controls were prepared by weighing out the corresponding peptide solid material and dissolving it in an appropriate vehicle (e.g. buffer or DMSO) to a final concentration of 1 mg/mL. Both, the control and sample were analyzed by reversed phase chromatography. The area under the peak of the sample was compared to the control and the solubility was calculated based on that ratio. The pH was measured and recorded for each sample.

UPLC method:

System: Waters UPLC
Mobile phase A: 0.1% trifluoracetic acid in water
Mobile phase B: 0.1% trifluoracetic acid in acetonitrile

| | | |
|---|---|---|
| Gradient: | | 95% mobile phase A, 5% mobile phase B (0 min) |
| | to | 5% mobile phase A, 95% mobile phase B (2 min) |
| | to | 5% mobile phase A, 95% mobile phase B (2.6 min) |
| | to | 95% mobile phase A, 5% mobile phase B (2.8 min) |

Injection volume: 3 μL and 0.3 μL
Flow: 0.8 mL/min
Column: ACQUITY UPLC BEH C18, 1.7 μm, 50 × 2.1 mm
Column temperature: 40° C.
Detection wavelength: 220 nm, 230 nm, 254 nm, 280 nm

The results for solubility are summarized in Table 7, below:

| Compound | Solubility | |
|---|---|---|
| No. | (mg/ml) | pH |
| 1 | 8.2 | 7.5 |
| 6 | 4.6 | 7.2 |
| 7 | 7.0 | 7.0 |
| 8 | 1.9 | 7.3 |
| 20 | 8.4 | 7.2 |
| 21 | 7.5 | 7.3 |
| 22 | 8.6 | 7.3 |
| 23 | 8.9 | 7.3 |
| 24 | 9.5 | 7.3 |
| 25 | 10.0 | 7.2 |
| 26 | 7.7 | 7.3 |
| 27 | 8.8 | 7.2 |
| 28 | 8.5 | 7.2 |
| 33 | 4.4 | 7.3 |
| 38 | 8.8 | 7.2 |
| 42 | 5.3 | 6.9 |
| 43 | 5.6 | 7.0 |
| 68 | 0.9 | 7.6 |
| 69 | 9.5 | 7.3 |
| 70 | 8.3 | 7.2 |
| 72 | 9.6 | 7.2 |

### Example 7: Chemical Stability

Peptides were dissolved in the test medium (50 mM HEPES buffer pH 7.4) to a concentration of 1 mg/mL. From each vial a sample was analyzed with reversed phase chromatography to obtain starting data (TO). The vials were incubated for the described time and temperature (14 days at 40° C.). Thereafter, a sample from each vial was analyzed with reversed phase chromatography (T1). Relative peak area values (% area) were obtained from corresponding chromatograms by normalizing the peak area of the peptide signal against the total peak area of the chromatogram for T0 and T1 measurements. Chemical stability of peptides was then calculated by subtracting T1% area values from T0% area values of respective peptides.

### UPLC Method:

System: Waters UPLC
Mobile phase A: 0.3% trifluoracetic acid in water
Mobile phase B: 0.3% trifluoracetic acid in acetonitrile

| | | |
|---|---|---|
| Gradient: | | 60% mobile phase A, 40% mobile phase B (0 min) |
| | to | 35% mobile phase A, 65% mobile phase B (28 min) |
| | to | 60% mobile phase A, 40% mobile phase B (28.1 min) |

Injection volume: 3 μL
Flow: 0.5 mL/min
Column: Kinetex C8, 2.6 μm, 100 Å, 150 × 4.6 mm
Column temperature: 50° C.
Detection wavelength: 220 nm, 277 nm

The retention time of the analyte peak should be in the range of 5 minutes to 20 minutes. If the standard method did not achieve a corresponding retention time, the mobile phase gradient was changed to achieve an appropriate retention time of the analyte.

The results for chemical stability are summarized in Table 8, below:

| | Chemical Stability, |
|---|---|
| Compound | % degradation |
| No. | (time, temperature) |
| 1 | 0.6 (14 days, 40° C.) |
| 3 | <0.1 (14 days, 40° C.) |
| 4 | <0.1 (14 days, 40° C.) |
| 6 | 6.5 (28 days, 25° C.) |
| 7 | 0.3 (14 days, 40° C.) |
| 9 | 0.9 (28 days, 25° C.) |
| 10 | 6.1 (28 days, 25° C.) |
| 21 | 2.1 (14 days, 40° C.) |
| 22 | <0.1 (14 days, 40° C.) |
| 23 | 9.1 (14 days, 40° C.) |
| 26 | 10 (14 days, 40° C.) |
| 27 | 9.6 (14 days, 40° C.) |
| 28 | 8.7 (14 days, 40° C.) |
| 33 | <0.1 (14 days, 40° C.) |
| 42 | 11.2 (14 days, 40° C.) |
| 43 | 1.9 (14 days, 40° C.) |
| 69 | 0.7 (14 days, 40° C.) |

### Example 8: Physical Stability

Physical instability of peptides in the form of amyloid fibril formation was detected using the amyloid-specific fluorescent dye Thioflavin T (ThT). Peptides were dissolved in buffer (50 mM HEPES buffer pH 7.4) and ThT was added to final concentrations of 1 mg/mL peptide and 40 NM ThT, respectively. Samples were then loaded into a 384-well black fluorescence plate (clear bottom) in triplicates (80 μL, each). Fluorescence intensity data were collected at fixed intervals of 10 minutes each preceded by 300 seconds of automixing (agitation) over a period of 96 hours at 40° C. using a fluorescence plate reader. Peptides were rated as physically instable if a substantial increase in total fluorescence intensity (>5× background) with a sigmoidal curve shape was observ-able over the course of the experiment.

### Fluorescence Measurement:

Extinction: 448 nm
Emission: 482 nm

The results for chemical stability are summarized in Table 9, below:

| Compound No. | |
|---|---|
| 1 | No fibrillation |
| 7 | No fibrillation |
| 20 | No fibrillation |
| 24 | No fibrillation |
| 25 | No fibrillation |
| 33 | No fibrillation |
| 42 | No fibrillation |
| 69 | No fibrillation |

### Example 9: Pharmacodynamics Effect on Acute Food Intake in Normal NMRI Mice

Male NMRI mice were obtained from Charles River (Charles River, Research Models & Services Germany GmbH) or from JanVier (JanVier Labs, France) at 3 weeks of age. The animals were group housed 4 mice pr. cage under a 12/12 hours dark-light cycle, light off at 3 pm. Room temperature was controlled to 21° C.±1° C., with 60%±20% humidity. Animals had ad libitum access to regular rodent chow (KLIBA Nafag 3430 or Altromin 1324, Brogaarden, Denmark) and tap water. Animals were transferred at 6 weeks of age, 5-7 days before the start of the study to a real-time food intake monitoring system, HM-2 system (MBRose, Denmark), to allow acclimatization to experimental conditions. As the animals were uniquely identified with microchips, each individual animal was identified by its microchip upon entry and exit from the food channel. Randomization of the mice for each study group (n=8-11) was based on body weight measured the day before the start of the study. A vehicle-treated (50 mM acetate buffer pH 7.4 with 3.5% mannitol) group was included in each experiment. Six hours before the start of the night phase animals were fasted. One hour before the dark phase animals were dosed once subcutaneously with test peptide. Food intake was reported hourly for a period of 24 hours. The food intake of the treated groups was normalized (in %) to the average food intake of the group receiving vehicle (table 10). Statistical significance was evaluated using One-way analysis of variance with Bonferroni's multiple comparison test. P<0.05 was considered statistically significant.

The results for reduction of acute food intake are summarized in Table 10, below:

| Compound | Dose | Acute Food Intake |
|---|---|---|
| No. | [nmol/kg] | 24 h [% vehicle] |
| 1 | 100 | 31% |
| 7 | 100 | 46% |
| 26 | 100 | 37% |
| 33 | 100 | 37% |
| 42 | 100 | 41% |
| 43 | 100 | 28% |
| 69 | 100 | 49% |

### Example 10: Pharmacodynamics Effect on Body Weight after Chronic Repeated Dosing in Diet-Induced Obese C57BL6 Mice of an NMUR2 Agonist of the Invention

Male C57BL6/J mice pre-fed with 60% high-fat diet are obtained from The Jackson Laboratories (Bar Harbour, ME, USA) at >16 weeks of age. Upon arrival, mice are single-housed to obtain accurate and individual food intake measurements for each animal. Animals are housed at room temperature of 21° C.±1° C., relative humidity 60±20% and a reversed 12-hours light/dark cycle (lights off at 10 am). During the entire study, animals have ad libitum access to food (high-fat diet D12492; Research Diets, Inc., New Brunswick, NJ, USA) and tap water. Before the start of the study, animals are randomized based on their bodyweight measured 1 week prior to the start of treatment (e.g. n=11/group). At the study start, the age of mice is >22 weeks. Mice are administered chronic repeated subcutaneous (SC) dosing of vehicle or an NMUR2 agonist of the present invention daily for 28 (or 14) days one hour before the dark cycle, with bodyweight and food intake measured every day. Body weight of the treated groups is normalized (in %) to the average body weight of the group receiving vehicle. Statistical significance is evaluated using One-way analysis of variance at day 28 (or 14) with Bonferonni's multiple comparison test. P<0.05 is considered statistically significant.

### Example 11: Pharmacodynamics Effect on Body Weight after Chronic Repeated Dosing in Diet-Induced Obese C57BL6 Mice of an NMUR2 Agonist of the Invention and a Further Active Agent Such as a GCCR/GLP-1R Dual Agonist

Male C57BL6/J mice pre-fed with 60% high-fat diet are obtained from The Jackson Laboratories (Bar Harbour, ME, USA) at >16 weeks of age. Upon arrival, mice are single-housed to obtain accurate and individual food intake measurements for each animal. Animals are housed at room temperature of 21° C.±1° C., relative humidity 60±20% and a reversed 12-h light/dark cycle (lights off at 10 am). During the entire study, animals have ad libitum access to food (high-fat diet D12492; Research Diets, Inc., New Brunswick, NJ, USA) and tap water. Before the start of the study, animals are randomized based on their bodyweight measured 1 week prior to the start of treatment (e.g. n=11/group). At the study start, the age of mice is greater than 22 weeks. Mice are administered chronic repeated SC dosing of vehicle, an NMUR2 agonist of the present invention, a further active agent such as a GCCR/GLP-1R dual agonist (preferably the dual agonist of formula (III)), or a combination of an NMUR2 agonist of the present invention and a further active agent daily for 28 days one hour before the dark cycle, with bodyweight and food intake measured every day. Body weight of the treated groups is normalized (in %) to the average body weight of the group receiving vehicle. Statistical significance was evaluated using One-way analysis of variance at day 14 with Bonferroni's multiple comparison test. P<0.05 is considered statistically significant.

### Reference Compounds

Reference compounds are described in Table 11, below:

| Reference | | SEQ. |
|---|---|---|
| Compound | Structure Reference | ID |
| No. | Compound | NO |
| Ref. 1 | C18A-gGlu₃-Sar₁₅-AEPipAc- | 81 |
| | Tyr-Phe-NMeLeu-Nle-Arg- | |
| | Pro-Arg-Gly-NH₂ | |
| Ref. 2 | C18DA-gGlu₃-Sar₁₅-AEPipAc- | 82 |
| | Tyr-Phe-Leu-Nle-Arg-Pro- | |
| | Arg-Gly-NH₂ | |
| Ref. 3 | C20A-gGlu₃-Sar₁₅-AEPipAc- | 83 |
| | Tyr-Phe-NMeLeu-Nle-Orn- | |
| | Pro-Arg-Gly-NH₂ | |
| Ref. 4 | C20DA-gGlu₃-Sar₁₅-AEPipAc- | 84 |
| | Tyr-Phe-Leu-Nle-Orn-Pro- | |
| | Arg-Gly-NH₂ | |
| Ref. 5 | C18A-gGlu₃-Sar₁₂-AEPipAc- | 85 |
| | Tyr-Phe-NMeLeu-Nle-Orn- | |
| | Pro-Arg-Gly-NH₂ | |
| Ref. 6 | C18DA-gGlu₃-Sar₁₂-AEPipAc- | 86 |
| | Tyr-Phe-Leu-Nle-Orn-Pro- | |
| | Arg-Gly-NH₂ | |

The structure of compound Ref. 1 is—except for the lipophilic substituent octadecanoyl (briefly C18A)—identical to Compound 7.

The structure of compound Ref. 2 is—except for leucine (Leu) at position 3 of peptide P—identical to Compound 7. Compound Ref. 2 doesn't comprise a N-methylleucine (NMeLeu) at position 3 of peptide P.

The structure of compound Ref. 3 is—except for the lipophilic substituent icosanoyl (briefly C20A)—identical to Compound 1.

The structure of compound Ref. 4 is—except for leucine (Leu) at position 3 of peptide P—identical to Compound 1 Compound Ref. 4 doesn't comprise a N-methylleucine (NMeLeu) at position 3 of peptide P.

The structure of compound Ref. 5 is—except for the lipophilic substituent octadecanoyl (briefly C18A)—identical to Compound 69.

The structure of compound Ref. 6 is—except for leucine (Leu) at position 3 of peptide P—identical to Compound 69 Compound Ref. 6 doesn't comprise a N-methylleucine (NMeLeu) at position 3 of peptide P.

The following compilation showcases the relative superiority with reference to the selectivity for the neuromedin U receptor 2 (NMUR2) over neuromedin U receptor 1 (NMUR1), i.e. selectivity according to activity as measured in Example 3 (EC₅₀[hNMUR1]/EC₅₀[hNMUR2]), of compounds of the present invention according to general formula (I) with respect to the corresponding reference compounds comprising the monocarboxylic acid half-life extension group (i.e. C18A or C20A) instead of the dicarboxylic acid half-life extension group (i.e. C18DA or C20DA) (see table 12A) or, alternatively, comprising a leucine (Leu) at position 3 of peptide P instead of a N-methylleucine (NMeLeu) (see table 12B).

Compounds of the present invention showed a higher selectivity for NMUR2 over NMUR1 with respect to the corresponding reference compounds.

TABLE 12A

| | | | EC₅₀ |
|---|---|---|---|
| | | SEQ | (hNMUR1)/ |
| | | ID | EC₅₀ |
| | Structure Compound | NO: | (hNMUR2) |
| Ref. 1 | C18A-gGlu₃-Sar₁₅-AEPipAc-Y- | 81 | 4.3 |
| | F-NMeL-Nle-R-P-R-G--NH₂ | | |
| Compound | C18DA-gGlu₃-Sar₁₅-AEPipAc-Y- | 7 | 208 |
| 7 | F-NMeL-Nle-R-P-R-G-NH₂ | | |
| Ref. 3 | C20A-gGlu₃-Sar₁₅-AEPipAc-Y- | 83 | 6.1 |
| | F-NMeL-Nle-Orn-P-R-G-NH₂ | | |
| Compound | C20DA-gGlu₃-Sar₁₅-AEPipAc-Y- | 1 | 635 |
| 1 | F-NMeL-Nle-Orn-P-R-G-NH₂ | | |
| Ref. 5 | C18A-gGlu₃-Sar₁₂-AEPipAc-Y- | 85 | 7.9 |
| | F-NMeL-Nle-Orn-P-R-G-NH₂ | | |
| Compound | C18DA-gGlu₃-Sar₁₂-AEPipAc-Y- | 69 | 508 |
| 69 | F-NMeL-Nle-Orn-P-R-G-NH₂ | | |

TABLE 12B

| | | | EC₅₀ |
|---|---|---|---|
| | | SEQ | (hNMUR1)/ |
| | | ID | EC₅₀ |
| | Structure Compound | NO: | (hNMUR2) |
| Ref. 2 | C18DA-gGlu₃-Sar₁₅-AEPipAc- | 82 | 12.4 |
| | Y-F-L-Nle-R-P-R-G-NH₂ | | |
| Compound 7 | C18DA-gGlu₃-Sar₁₅-AEPipAc- | 7 | 208 |
| | Y-F-NMeL-Nle-R-P-R-G-NH₂ | | |
| Ref. 4 | C20DA-gGlu₃-Sar₁₅-AEPipAc- | 84 | 25.0 |
| | Y-F-L-Nle-Orn-P-R-G -NH₂ | | |
| Compound 1 | C20DA-gGlu₃-Sar₁₅-AEPipAc- | 1 | 635 |
| | Y-F-NMeL-Nle-Orn-P-R-G-NH₂ | | |
| Ref. 6 | C18DA-gGlu₃-Sar₁₂-AEPipAc- | 86 | 32.4 |
| | Y-F-L-Nle-Orn-P-R-G-NH₂ | | |
| Compound69 | C18DA-gGlu₃-Sar₁₂-AEPipAc- | 69 | 508 |
| | Y-F-NMeL-Nle-Orn-P-R-G-NH₂ | | |

Ref. 1: EC₅₀(hNMUR1)/EC₅₀(hNMUR2)=4.3, according to the respective activity on hNMUR1 (EC₅₀=0.2 nM) and on NMUR2 (EC₅₀=0.046 nM).
Ref. 2: EC₅₀(hNMUR1)/EC₅₀(hNMUR2)=12.4, according to the respective activity on hNMUR1 (EC₅₀=21.0 nM) and on hNMUR2 (EC₅₀=1.7 nM).
Ref 3: EC₅₀(hNMUR1)/EC₅₀(hNMUR2)=6.1, according to the respective activity on hNMUR1 (EC₅₀=0.33 nM) and on hNMUR2 (EC₅₀=0.054 nM).
Ref 4: EC₅₀(hNMUR1)/EC₅₀(hNMUR2)=25.0, according to the respective activity on hNMUR1 (EC₅₀=75.0 nM) and on hNMUR2 (EC₅₀=3.0 nM).
Ref 5: EC₅₀(hNMUR1)/EC₅₀(hNMUR2)=7.9, according to the respective activity on hNMUR1 (EC₅₀=0.37 nM) and on hNMUR2 (EC₅₀=0.047 nM).
Ref 6: EC₅₀(hNMUR1)/EC₅₀(hNMUR2)=32.4, according to the respective activity on hNMUR1 (EC₅₀=68 nM) and on hNMUR2 (EC₅₀=2.1 nM).
Compound 7: EC₅₀(hNMUR1)/EC₅₀(hNMUR2)=208, according to the respective activity on hNMUR1 (EC₅₀=250 nM) and on hNMUR2 (EC₅₀=1.2 nM).
Compound 1: EC₅₀(hNMUR1)/EC₅₀(hNMUR2)=635 for Compound 1, according to the respective activity on hNMUR1 (EC₅₀=890 nM) and on hNMUR2 (EC₅₀=1.4 nM).
Compound 69: EC₅₀(hNMUR1)/EC₅₀(hNMUR2)=508 for Compound 69, according to the respective activity on hNMUR1 (EC₅₀=660 nM) and on hNMUR2 (EC₅₀=1.3 nM).

The following compilation showcases the relative superiority with reference to the prolonged half-life, as determined by mean residence time (MRT) after intravenous application as in Example 5, of compounds of the present invention according to general formula (I) with respect to the corresponding reference compounds comprising the monocarboxylic acid half-life extension group (i.e. C18A or C20A) instead of the dicarboxylic acid half-life extension group (i.e. C18DA or C20DA) (see table 13A) or, alternatively, comprising a leucine (Leu) at position 3 of peptide P instead of a N-methylleucine (NMeLeu) (see table 13B).

Compounds of the present invention showed a prolonged half-life with respect to the corresponding reference compounds.

TABLE 13A

| | | SEQ | |
|---|---|---|---|
| | | ID | MRT |
| | Structure Compound | NO: | (hours) |
| Ref. 1 | C18A-gGlu₃-Sar₁₅-AEPipAc-Y- | 81 | 0.8 |
| | F-NMeL-Nle-R-P-R-G-NH₂ | | |
| Compound | C18DA-gGlu₃-Sar₁₅-AEPipAc- | 7 | 17.4 |
| 7 | Y-F-NMeL-Nle-R-P-R-G-NH₂ | | |
| Ref. 3 | C20A-gGlu₃-Sar₁₅-AEPipAc-Y- | 83 | 1.7 |
| | F-NMeL-Nle-Orn-P-R-G-NH₂ | | |
| Compound | C20DA-gGlu₃-Sar₁₅-AEPipAc- | 1 | 38.1 |
| 1 | Y-F-NMeL-Nle-Orn-P-R-G-NH₂ | | |
| Ref. 5 | C18A-gGlu₃-Sar₁₂-AEPipAc-Y- | 85 | 2.2 |
| | F-NMeL-Nle-Orn-P-R-G-NH₂ | | |
| Compound | C18DA-gGlu₃-Sar₁₂-AEPipAc- | 69 | 20.2 |
| 69 | Y-F-NMeL-Nle-Orn-P-R-G-NH₂ | | |

TABLE 13B

| | | SEQ | |
|---|---|---|---|
| | | ID | MRT |
| | Structure Compound | NO: | (hours) |
| Ref. 2 | C18DA-gGlu₃-Sar₁₅-AEPipAc- | 82 | 7.9 |
| | Y-F-L-Nle-R-P-R-G-NH₂ | | |
| Compound | C18DA-gGlu₃-Sar₁₅-AEPipAc- | 7 | 17.4 |
| 7 | Y-F-NMeL-Nle-R-P-R-G-NH₂ | | |
| Ref. 4 | C20DA-gGlu₃-Sar₁₅-AEPipAc- | 84 | 22.5 |
| | Y-F-L-Nle-Orn-P-R-G-NH₂ | | |
| Compound | C20DA-gGlu₃-Sar₁₅-AEPipAc- | 1 | 38.1 |
| 1 | Y-F-NMeL-Nle-Orn-P-R-G-NH₂ | | |
| Ref. 6 | C18DA-gGlu₃-Sar₁₂-AEPipAc- | 86 | 4.2 |
| | Y-F-L-Nle-Orn-P-R-G-NH₂ | | |
| Compound | C18DA-gGlu₃-Sar₁₂-AEPipAc- | 69 | 20.2 |
| 69 | Y-F-NMeL-Nle-Orn-P-R-G-NH₂ | | |

### Experimental Section

### List Abbreviations

ACN: acetonitrile
Boc: tert butyloxycarbonyl-
DCM: dichloromethane
DIC: diisopropylcarbodiimide
DMF: N,N-dimethylformamide
Fmoc: 9H-fluoren-9-ylmethoxycarbonyl-
m/z: mass to charge ratio (MS signal); z is an integer
NMP: N-methyl-2-pyrrolidone
oxyma: ethyl cyano(hydroxyimino)acetate
Pbf: 2,2,4,6,7-pentamethyl-2,3-dihydro-1-benzofuran-5-sulfonyl-
Pfp: pentafluorophenyl-
Rt: retention time
RT: room temperature
tBu: tert butyl-
TFA: trifluoroacetic acid
Trt: triphenylmethyl-(trityl-)

### General Procedure for Solid Phase Synthesis of Peptides

All peptides were synthesized by standard Fmoc-solid phase peptide chemistry on a Tentagel S RAM resin (loading 0.2-0.4 mmol/g, 90 μM) supplied by Rapp Polymere GmbH.

The following protected amino acids were used: Fmoc-Arg(Pbf)-OH, Fmoc-Dbu(Boc)-OH, Fmoc-Glu-OtBu, Fmoc-Gly-OH, Fmoc-His(Trt)-OH, Fmoc-Lys(Boc)-OH, Fmoc-hLys(Boc)-OH, Fmoc-NMeL-OH, Fmoc-NMeA-OH, Fmoc-Orn(Boc)-OH, Fmoc-2Pal-OH, Fmoc-3Pal-OH, Fmoc-4Pal-OH, Fmoc-Nle-OH, Fmoc-Phe-OH, Fmoc-Pro-OH, Fmoc-Tyr(tBu)-OH, Fmoc-PipAc-OH, Fmoc-AEPipAc-OH-2 HCl. The L-form of the amino acid building blocks was utilized if not specified otherwise. The modular half-life extension moiety was built up by SPPS (solid phase peptide synthesis) using protected building blocks such as, but not limited to, stearic acid (C18A), icosanoic acid (arachidic acid, C20A), 18-(tert-butoxy)-18-oxooctadecanoic acid (C18DA(tBu)), 20-(tert-butoxy)-20-oxoicosanoic acid (C20DA(tBu)), Fmoc-Glu-OtBu, Fmoc-Sar-OH, Fmoc-Sar3-OH, and Fmoc-Sar-OPfp.

The amino acid building blocks, Fmoc-Glu-OtBu, oxyma, DIC, and all reagents were purchased from standard suppliers, e.g. Bachem A G, Merck KGaA (Novabiochem®, Sigma-Aldrich®), ABCR GmbH, Iris Biotec GmbH. 18-(Tert-butoxy)-18-oxooctadecanoic acid (C18DA(OtBu)) was purchased from Cool Pharm Ltd., 20-(tert-butoxy)-20-oxoicosanoic acid (C20DA(OtBu)) was purchased from Angene and icosanoic acid was purchased from EGA-Chemie.

The assembly of the peptides was started at the C-terminus by stepwise chain elongation according to the respective sequences by alternating Fmoc-deblocking and coupling steps.

The NMU-8 analogues were obtained as TFA salts from the cleavage/deprotection or from the HPLC purification. The trifluoroacetate can be exchanged by common procedures, such as resin-ion exchange procedures, e.g. as disclosed in Roux, St. et al. J. Pept. Sci. 2008; 14: 354-359.

### Synthesis Method (S01)

Peptides were synthesized by microwave assisted solid phase peptide synthesis (SPPS) on a CEM Liberty Blue Peptide Synthesizer at 0.10 mmol-0.25 mmol scale on Rink amide resin using Fmoc strategy. Coupling of amino acids was affected by addition of DMF solutions of suitably protected amino acid building block (0.2 mol/I, 4-5 eq), oxyma (1 mol/I, 4-5 eq) and DIC (1 mol/I, 1 ml, 8-10 eq) to the resin and heating of the resulting suspension to 90° C. by microwave irradiation for 4 minutes (min). For the coupling of Fmoc-Arg(Pbf)-OH, Fmoc-Pro-OH, and fatty acid building blocks (C18DA, C18A, C20DA, C20A) the coupling cycle was repeated (double coupling, 2×4 min at 90° C.). Fmoc-PipAc-OH, Fmoc-AEPipAc-OH·2 HCl, Fmoc-2Pal-OH, Fmoc-3Pal-OH, Fmoc-4Pal-OH, Fmoc-NMeL-OH, and Fmoc-NMeA-OH were coupled at 75° C. for 20 min. In some instances, Fmoc-AEPipAc-OH·2 HCl was coupled at 90° C. (1×20 min). Sar was introduced using Fmoc-Sar-OH, Fmoc-Sar-OH or Fmoc-Sar-OPfp (coupling at 75° C. for 4 or 20 min). Fmoc-Glu-OtBu was coupled two times at 75° C. for 20 min. Fmoc-His(Trt)-OH was coupled at 50° C. (2×12 min). Amino acids following NMeL or NMeA were introduced by double coupling. Prior to the conjugation of the fatty acid building block in the final coupling cycle, the peptidyl resin was treated with acetic anhydride for 5 min at 65° C. (capping). Removal of the Fmoc protective group was performed using an excess of 10% piperidine/DMF for 1 min at 90° C.; this step was repeated for the removal of the first Fmoc group.

After completion of peptide chain assembly, the resin was carefully washed with DCM and dried prior to cleavage. Concomitant resin cleavage and side chain deprotection was performed using a mixture of TFA/water/triisopropylsilane (95:5:2.5) for 45 min at 40° C. Crude peptides were precipitated with cold diethyl ether, dissolved in 50% acetonitrile/water and purified by preparative HPLC (P01 or P02).

### Synthesis Method (S02)

In an equivalent fashion, peptides were synthesized at elevated temperatures on a PurePep® Chorus (Gyros Protein Technologies) automated peptide synthesizer. For peptide chain elongation suitably protected building blocks, dissolved in DMF (0.2 mol/I, 4 eq) were added to the solid phase resin in the reaction vessel and treated with DMF solutions of oxyma (1 mol/I, 4 eq) and DIC (1 mol/I, 8 eq). The resulting suspension was heated and kept at a final temperature of 85° C. for 4 min. Fmoc-Arg(Pbf)-OH and fatty acid building block (C20DA,) were introduced by double coupling (2×4 min at 85° C.). Fmoc-Glu-OtBu, Fmoc-AEPipAc-OH·2 HCl, Fmoc-Sar-OH, and Fmoc-NMeL-OH were coupled at 75° C. for 20 min. Amino acids following NMeL or NMeA were introduced by double coupling. Prior to the conjugation of the fatty acid building block in the final coupling cycle, the peptidyl resin was treated with acetic anhydride for 5 min at 65° C. (capping). Removal of the Fmoc protective group was performed using an excess of 10% piperidine/DMF for 1 min at 85° C.; this step was repeated for the removal of the first Fmoc group.

### Synthesis Method (S03)

Alternatively, peptides were synthesized on an in house designed fully automated parallel peptide synthesizer based on a liquid handling platform (TECAN AG, Switzerland) and equipped with temperature-controlled heating blocks. Peptide chain assembly was achieved by addition of DMF solutions of suitably protected building blocks (0.2 mol/I, 4-6 eq.), oxyma (1 mol/I, 4-6 eq.) and DIC (1 mol/I, 8-12 eq.) to the reaction vessels containing aliquots of the resin (0.05 mmol-0.1 mmol scale). The resulting suspensions were agitated by intermittent shaking (intervals of 12 s, every 2 min) and heated for 10 min at 75° C. Fmoc-PipAc-OH, Fmoc-AEPipAc-OH 2 HCl, Fmoc-2Pal-OH, Fmoc-3Pal-OH, Fmoc-4Pal-OH, were coupled 40 min at 75° C. Fmoc-Arg(Pbf)-OH, and fatty acid building blocks (C18DA, C20DA) were introduced by double coupling (2×10 min at 75° C.).

Fmoc-Glu-OtBu, Fmoc-NMeL-OH, and Fmoc-NMeA-OH were coupled for 2×40 min at 75° C.

Fmoc-His(Trt)-OH was coupled 2×20 min at 50° C. Amino acids following NMeL or NMeA were introduced by double coupling. Capping with acetic anhydride (20% in DMF, 1.5 ml) was performed prior to the conjugation with the fatty acid in the final coupling step. For Fmoc-deprotection, the resin was treated with 10% piperidine in DMF (1.0 mL) at elevated temperature (75° C.) for 5 min; in the first cycle, piperidine treatment was repeated for additional 5 min.

For liberation of the fully deprotected peptide from the solid support the resin was washed with DCM (5×2.5 ml), dried and treated with a mixture of 95% TFA (2.5 ml) and triisopropylsilane (0.05 ml) for 30 min at 40° C. The crude peptide solutions were drained, combined with two additional washings (2×2 mL 95% TFA) and concentrated under reduced pressure. The residues were re-dissolved in DMF/ACN/water.

### Purification Method (P01)

Crude peptides were dissolved in DMF/acetonitrile/water and purified by reversed phase chromatography using an Agilent preparative HPLC-MS System with preparative pumps G1361A, a diode array detector G1315B, a mass-spectrometer G1956B and a fraction collector CTC PAL IFC. Stationary phase was a Waters XSelect CSH Prep column (phenyl hexyl, 5 μm, 30×150 mm). The peptides were eluted with a linear gradient of eluent A (0.1% TFA in H₂O) and eluent B (ACN) at a flow rate of 50 ml/min and a temperature of 40° C. Homogeneous fractions were pooled and lyophilized. The final product was characterized by HPLC-MS (A01, A02, A03, or A04).

### Purification Method (P02)

Crude peptides were dissolved in DMF/acetonitrile/water (e.g. NMP) and purified by reversed phase chromatography using an Agilent preparative HPLC-MS System with preparative pumps G7161B, G7111B, G7110B, a diode array detector G7115A, a mass-spectrometer G6135B and a autosampler/fraction collector G7159B. Stationary phase was a Phenomenex LUNA C8 10 μm Prep Column (50×250 mm). The peptides were eluted with a linear gradient of eluent A (0.1% TFA in H₂O) and eluent B (0.1% TFA in ACN) at a flow rate of 120 ml/min and a temperature of 40° C. Homogeneous fractions were pooled and lyophilized. The final product was characterized by HPLC-MS (A03, A04).

### Analytical Methods (A01, A02, A03, and A04)

Peptide purity and mass were estimated by analytical HPLC-MS on a Kinetex C8 column (4.6 mm×150 mm, 2.6 um, Phenomenex) using a Waters Acquity HPLC System equipped with 3100 Mass Detector (A01, A02) or Agilent 1260 HPLC system equipped with Mass Detector G6135 (A03) or Agilent 1290 HPLC system equipped with Time-of-Flight (TOF) MS G6230 (A04). Analysis was performed by gradient elution with eluent A (0.3% TFA in H₂O) and eluent B (0.24% TFA in ACN) at a temperature of 40° C.

Details of the gradient and flow rates are summarized in table 14, 15, 16, and 17. Chromatographic retention times and most prominent m/z signals (+ESI mode) were recorded, where m=molecular mass and z=net charge of the observed molecular species.

Peptide purities (relative peak areas @ 214 nm) were in the range from 80% to 99%, preferably greater than 95%.

TABLE 14

| | |
|---|---|
| Method Name: | A01 |
| Device: | Waters Acquity with 3100 MS |
| Column: | Phenomenex Kinetex C8, 4.6 × 150 mm; 2.6 μm |

Gradient:

| | % Eluent A | % Eluent B | | |
|---|---|---|---|---|
| Time | [Water 0.3% | [ACN 0.24% | Flow | Temp |
| [min] | TFA (v/v)] | TFA (v/V)] | [ml/min] | [° C.] |
| 0.0 | 65.0 | 35.0 | 0.5 | 40.0 |
| 15.0 | 45.0 | 55.0 | 0.5 | 40.0 |
| 16.0 | 10.0 | 90.0 | 1.0 | 40.0 |
| 17.0 | 65.0 | 35.0 | 1.0 | 40.0 |
| 18.0 | 65.0 | 35.0 | 1.0 | 40.0 |

TABLE 15

| | |
|---|---|
| Method Name: | A02 |
| Device: | Waters Acquity with 3100 MS |
| Column: | Phenomenex Kinetex C8, 4.6 × 150 mm; 2.6 μm |

Gradient:

| | % Eluent A | % Eluent B | | |
|---|---|---|---|---|
| Time | [Water 0.3% | [ACN 0.24% | Flow | Temp |
| [min] | TFA (v/v)] | TFA (v/v)] | [ml/min] | [° C.] |
| 0.0 | 85.0 | 15.0 | 0.5 | 40.0 |
| 50.0 | 35.0 | 65.0 | 0.5 | 40.0 |
| 52.0 | 10.0 | 90.0 | 1.0 | 40.0 |
| 53.0 | 85.0 | 15.0 | 1.0 | 40.0 |
| 55.0 | 85.0 | 15.0 | 1.0 | 40.0 |

TABLE 16

| | |
|---|---|
| Method Name: | A03 |
| Device: | Agilent 1260 with MS G6135 |
| Column: | Phenomenex Kinetex C8, 4.6 × 150 mm; 2.6 μm |

Gradient:

| | % Eluent A | % Eluent B | | |
|---|---|---|---|---|
| Time | [Water 0.3% | [ACN 0.24% | Flow | Temp |
| [min] | TFA (v/v)] | TFA (v/v)] | [ml/min] | [° C.] |
| 0.0 | 85.0 | 15.0 | 0.5 | 40.0 |
| 50.0 | 35.0 | 65.0 | 0.5 | 40.0 |
| 52.0 | 10.0 | 90.0 | 1.0 | 40.0 |
| 53.0 | 85.0 | 15.0 | 1.0 | 40.0 |
| 55.0 | 85.0 | 15.0 | 1.0 | 40.0 |

TABLE 17

| | |
|---|---|
| Method Name: | A04 |
| Device: | Agilent 1290 with TOF-MS G6230B |
| Column: | Phenomenex Kinetex C8, 4.6 × 150 mm; 2.6 μm |

Gradient:

| | % Eluent A | % Eluent B | | |
|---|---|---|---|---|
| Time | [Water 0.3% | [ACN 0.24% | Flow | Temp |
| [min] | TFA (v/v)] | TFA (v/v)] | [ml/min] | [° C.] |
| 0.0 | 85.0 | 15.0 | 0.5 | 40.0 |
| 50.0 | 35.0 | 65.0 | 0.5 | 40.0 |
| 52.0 | 10.0 | 90.0 | 1.0 | 40.0 |
| 53.0 | 85.0 | 15.0 | 1.0 | 40.0 |
| 55.0 | 85.0 | 15.0 | 1.0 | 40.0 |

The following compounds were synthesized. All compounds were obtained as TFA salts:

### Compound 1

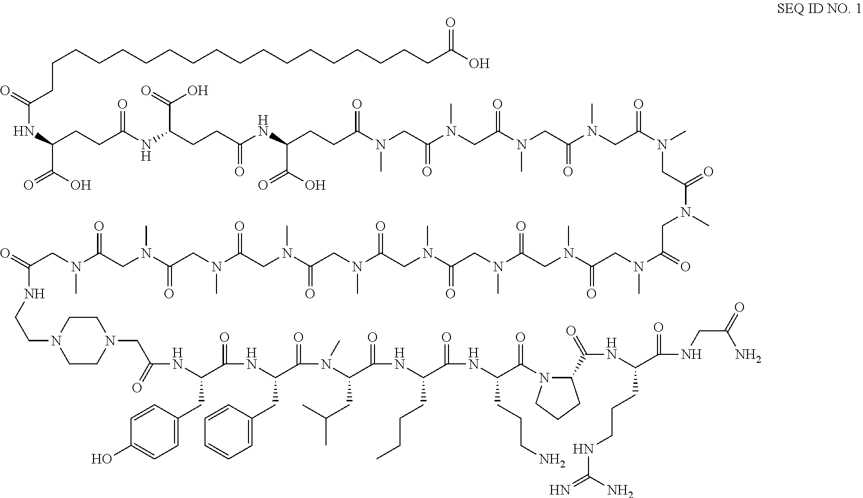 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCCCC(=O)O)C(=O)O)C(=O)O)C(=O)O)CC1)C(=O)N[C@@H](CCCN)C(=O)N1CCC[C@H]1C(=O)N[C@@H](CCCNC(=N)N)C(=O)NCC(N)=O] [0372] MW (calculated): 2939.50 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³, Nle⁴,Orn⁵,G⁸]NMU-8 (SEQ ID NO: 1)

(SEQ ID NO: 1)
C20DA-gGlu-gGlu-gGlu-Sar-Sar-Sar-Sar-Sar-
Sar-Sar-Sar-Sar-Sar-Sar-Sar-Sar-Sar-Sar-
AEPipAc-Y-F-NMeL-Nle-Orn-P-R-G-NH₂

Synthesis and purification methods: S01; P01
LCMS: A02
Rt: 32.4 min
m/2: 1469.8

### Compound 2

### SEQ ID NO. 2

MW (calculated): 2869.37 Da
2-[4-(2-(17-carboxyheptadecanoyl-L-γ-glutamyl-L-γ-glutamyl-L-γ-glutamyl-L-γ-glutamyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 2)

(SEQ ID NO: 2)
C18DA-gGlu-gGlu-gGlu-gGlu-Sar-Sar-Sar-Sar-
Sar-Sar-Sar-Sar-Sar-Sar-Sar-Sar-AEPipAc-Y-
F-NMeL-Nle-R-P-R-G-NH₂

Synthesis and purification methods: S01; P01
LCMS: A04
Rt: 28.6 min
m/3: 957.2

### Compound 3

### SEQ ID NO. 3

MW (calculated): 3068.62 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,Orn5,G⁸]NMU-8 (SEQ ID NO: 3)

(SEQ ID NO: 3)
C20DA-gGlu-gGlu-gGlu-gGlu-Sar-Sar-Sar-Sar-
Sar-Sar-Sar-Sar-Sar-Sar-Sar-Sar-Sar-Sar-
Sar-AEPipAc-Y-F-NMeL-Nle-Orn-P-R-G-NH₂

Synthesis and purification methods: S01; P01
LCMS: A04
Rt: 31.1 min
m/3: 1023.6

### Compound 4

### SEQ ID NO. 4

MW (calculated): 3054.59 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,Dbu⁵,G⁸]NMU-8 (SEQ ID NO: 4)

(SEQ ID NO: 4)
C20DA-gGlu-gGlu-gGlu-gGlu-Sar-Sar-Sar-Sar-
Sar-Sar-Sar-Sar-Sar-Sar-Sar-Sar-Sar-Sar-
Sar-AEPipAc-Y-F-NMeL-Nle-Dbu-P-R-G-NH₂

Synthesis and purification methods: S01; P01
LCMS: A04
Rt: 31.1 min
m/3: 1019.0

### Compound 5

### SEQ ID NO. 5

MW (calculated): 2827.32 Da
2-[4-(2-(17-carboxyheptadecanoyl-L-γ-glutamyl-L-γ-glutamyl-L-γ-glutamyl-L-γ-glutamyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,G⁸]NMU-8 (SEQ ID NO: 5)

(SEQ ID NO: 5)
C18DA-gGlu-gGlu-gGlu-Glu-Sar-Sar-Sar-Sar-
Sar-Sar-Sar-Sar-Sar-Sar-Sar-Sar-AEPipAc-
Y-F-NMeL-Nle-Orn-P-R-G-NH₂

Synthesis and purification methods: S01; P01
LCMS: A03
Rt: 29 min
m/3: 943.2

### Compound 6

### SEQ ID NO. 6

MW (calculated): 2768.30 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 6)

(SEQ ID NO: 6)
C20DA-gGlu-gGlu-gGlu-Sar-Sar-Sar-Sar-Sar-
Sar-Sar-Sar-Sar-Sar-Sar-Sar-AEPipAc-Y-F-
NMeL-Nle-R-P-R-G-NH₂

Synthesis and purification methods: S03; P01
LCMS: A01
Rt: 10.8 min
m/3: 923.7

### Compound 7

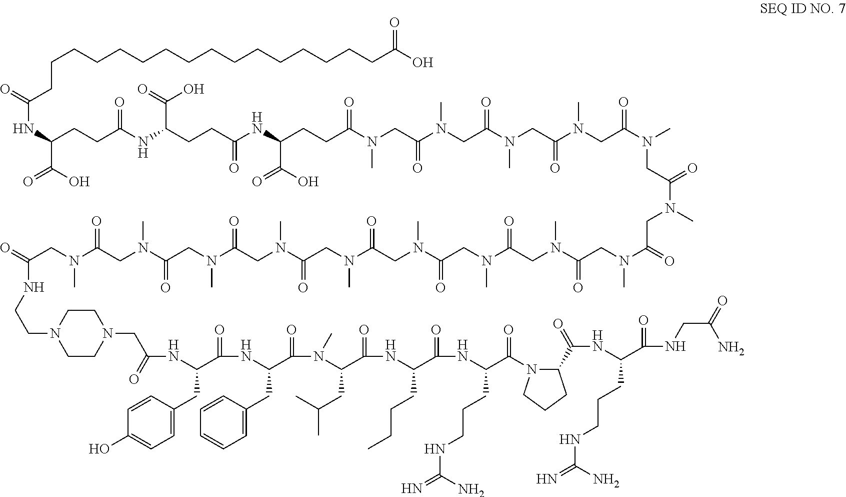 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCC(=O)O)C(=O)O)C(=O)O)C(=O)O)CC1)C(=O)N[C@@H](CCCNC(=N)N)C(=O)N1CCC[C@H]1C(=O)N[C@@H](CCCNC(=N)N)C(=O)NCC(N)=O] [0409] MW (calculated): 2953.49 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,G⁸]NMU-8 (SEQ ID NO: 7)

(SEQ ID NO: 7)
C18DA-gGlu-gGlu-gGlu-Sar-Sar-Sar-Sar-Sar-
Sar-Sar-Sar-Sar-Sar-Sar-Sar-Sar-Sar-Sar-
AEPipAc-Y-F-NMeL-Nle-R-P-R-G-NH₂

Synthesis and purification methods: S01; P02
LCMS: A03
Rt: 29.3 min
m/3: 985.2

### Compound 8

### SEQ ID NO. 8

MW (calculated): 2712.24 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,G⁸]NMU-8 (SEQ ID NO: 8)

(SEQ ID NO: 8)
C20DA-gGlu-gGlu-gGlu-Sar-Sar-Sar-Sar-Sar-
Sar-Sar-Sar-Sar-Sar-Sar-Sar-AEPipAc-Y-F-
NMeL-Nle-Dbu-P-R-G-NH₂

Synthesis and purification methods: S01; P01
LCMS: A03
Rt: 32.7 min
m/2: 1356.7

### Compound 9

### SEQ ID NO. 9

MW (calculated): 2981.54 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,G⁸]NMU-8 (SEQ ID NO: 9)

(SEQ ID NO: 9)
C20DA-gGlu-gGlu-gGlu-Sar-Sar-Sar-Sar-Sar-
Sar-Sar-Sar-Sar-Sar-Sar-Sar-Sar-Sar-Sar-
AEPipAc-Y-F-NMeL-Nle-R-P-R-G-NH₂

Synthesis and purification methods: S01; P01
LCMS: A03
Rt: 32.5 min
m/3: 994.6

### Compound 10

### SEQ ID NO. 10

MW (calculated): 2969.53 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methyl glycyl-N-methylglycyl-N-methylglycyl-N-methylglycyl-N-methylglycyl-N-methylglycylamino)ethyl)piper-azin-1-yl]acetyl-[NMeL³,Nle⁴,NMeA⁶,G⁸]NMU-8 (SEQ ID NO: 10)

(SEQ ID NO: 10)
C20DA-gGlu-Glu-gGlu-Sar-Sar-Sar-Sar-Sar-
Sar-Sar-Sar-Sar-Sar-Sar-Sar-Sar-Sar-Sar-
AEPipAc-Y-F-NMeL-Nle-R-NMeA-R-G-NH₂

Synthesis and purification methods: S01; P01
LCMS: A03
Rt: 32.5 min
m/2: 1485.4

### Compound 11

### SEQ ID NO. 11

MW (calculated): 2941.48 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,NMeA⁶,G⁸]NMU-8 (SEQ ID NO: 11)

(SEQ ID NO: 11)
C18DA-gGlu-gGlu-gGlu-Sar-Sar-Sar-Sar-Sar-
Sar-Sar-Sar-Sar-Sar-Sar-Sar-Sar-Sar-Sar-
AEPipAc-Y-F-NMeL-Nle-R-NMeA-R-G-NH₂

Synthesis and purification methods: S01; P01
LCMS: A02
Rt: 28.5 min
m/3: 982.5

### Compound 12

### SEQ ID NO. 12

MW (calculated): 2728.24 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,NMeA⁶,G⁸]NMU-8 (SEQ ID NO: 12)

(SEQ ID NO: 12)
C18DA-gGlu-gGlu-gGlu-Sar-Sar-Sar-Sar-Sar-
Sar-Sar-Sar-Sar-Sar-Sar-Sar-AEPipAc-Y-F-
NMeL-Nle-R-NMeA-R-G-NH₂

Synthesis and purification methods: S01; P01
LCMS: A02
Rt: 28.9 min
m/3: 911.4

### Compound 13

### SEQ ID NO. 13

MW (calculated): 2843.37 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,NMeA⁶,G⁸]NMU-8 (SEQ ID NO: 13)

(SEQ ID NO: 13)
C20DA-gGlu-gGlu-gGlu-gGlu-Sar-Sar-Sar-Sar-
Sar-Sar-Sar-Sar-Sar-Sar-Sar-Sar-AEPipAc-Y-
F-NMeL-Nle-Orn-NMeA-R-G-NH₂

Synthesis and purification methods: S03; P01
LCMS: A01
Rt: 10.3 min
m/3: 949.8

### Compound 14

### SEQ ID NO. 14

MW (calculated): 3056.60 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,Orn⁵,NMeA⁶,G⁸]NMU-8 (SEQ ID NO: 14)

(SEQ ID NO: 14)
C20DA-gGlu-gGlu-gGlu-gGlu-Sar-Sar-Sar-Sar-
Sar-Sar-Sar-Sar-Sar-Sar-Sar-Sar-Sar-Sar-Sar-
AEPipAc-Y-F-NMeL-Nle-Orn-NMeA-R-G-NH₂

Synthesis and purification methods: S03; P01
LCMS: A01
Rt: 10.0 min
m/3: 1020.9

### Compound 15

### SEQ ID NO. 15

MW (calculated): 2841.35 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,G⁸]NMU-8 (SEQ ID NO: 15)

(SEQ ID NO: 15)
C20DA-gGlu-gGlu-gGlu-gGlu-Sar-Sar-Sar-Sar-
Sar-Sar-Sar-Sar-Sar-Sar-Sar-Sar-AEPipAc-Y-
F-NMeL-Nle-Dbu-P-R-G-NH₂

Synthesis and purification methods: S03; P01
LCMS: A01
Rt: 10.3 min
m/3: 949.1

### Compound 16

### SEQ ID NO. 16

MW (calculated): 3042.58 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,Dbu⁵,NMeA⁶,G⁸]NMU-8 (SEQ ID NO: 16)

(SEQ ID NO: 16)
C20DA-gGlu-gGlu-gGlu-gGlu-Sar-Sar-Sar-Sar-Sar-Sar-
Sar-Sar-Sar-Sar-Sar-Sar-Sar-Sar-Sar-AEPipAc-Y-F-
NMeL-Nle-Dbu-NMeA-R-G-NH₂

Synthesis and purification methods: S03; P01
LCMS: A01
Rt: 10.1 min
m/3: 1016.2

### Compound 17

### SEQ ID NO. 17

MW (calculated): 3082.64 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,K⁵,G⁸]NMU-8 (SEQ ID NO: 17)

(SEQ ID NO: 17)
C20DA-gGlu-gGlu-gGlu-gGlu-Sar-Sar-Sar-Sar-Sar-Sar-
Sar-Sar-Sar-Sar-Sar-Sar-Sar-Sar-Sar-AEPipAc-Y-F-
NMeL-Nle-K-P-R-G-NH₂

Synthesis and purification methods: S03; P01
LCMS: A01
Rt: 10.0 min
m/3: 1029.6

### Compound 18

### SEQ ID NO. 18

MW (calculated): 3070.63 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl-N-methylglycyl-N-methylglycyla-mino)ethyl)piperazin-1-yl]acetyl-[NMeL³,Nle⁴,K⁵,NMeA⁶,G⁸]NMU-8 (SEQ ID NO: 18)

(SEQ ID NO: 18)
C20DA-gGlu-gGlu-gGlu-gGlu-Sar-Sar-Sar-Sar-Sar-Sar-
Sar-Sar-Sar-Sar-Sar-Sar-Sar-Sar-Sar-AEPipAc-Y-F-
NMeL-Nle-K-NMeA-R-G-NH₂

Synthesis and purification methods: S03; P01
LCMS: A01
Rt: 10.0 min
m/3: 1024.4

### Compound 19

### SEQ ID NO. 19

MW (calculated): 2857.39 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,K⁵,NMeA⁶,G⁸]NMU-8 (SEQ ID NO: 19)

(SEQ ID NO: 19)
C20DA-gGlu-gGlu-gGlu-gGlu-Sar-Sar-Sar-Sar-Sar-Sar-
Sar-Sar-Sar-Sar-Sar-Sar-AEPipAc-Y-F-NMeL-Nle-K-
NMeA-R-G-NH₂

Synthesis and purification methods: S03; P01
LCMS: A01
Rt: 10.3 min
m/3: 954.5

### Compound 20

### SEQ ID NO. 20

MW (calculated): 2686.20 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,NMeA⁶,G⁸]NMU-8 (SEQ ID NO: 20)

(SEQ ID NO: 20)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-Orn-NMeA-
R-G-NH₂

Synthesis and purification methods: S01; P01
LCMS: A03
Rt: 29.7 min
m/2: 1343.6

### Compound 21

### SEQ ID NO. 21

MW (calculated): 2911.45 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Orn⁵,G⁸]NMU-8 (SEQ ID NO: 21)

(SEQ ID NO: 21)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-P-R-G-NH₂

Synthesis and purification methods: S01; P01
LCMS: A02
Rt: 28.6 min
m/3: 972.7

### Compound 22

### SEQ ID NO. 22

MW (calculated): 2899.44 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Orn⁵,NMeA⁶,G⁸]NMU-8 (SEQ ID NO: 22)

(SEQ ID NO: 22)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-NMeA-R-G-NH₂

Synthesis and purification methods: S01; P01
LCMS: A02
Rt: 28.5 min
m/3: 968.6

### Compound 23

### SEQ ID NO. 23

MW (calculated): 2885.41 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Dbu⁵,NMeA⁶,G⁸]NMU-8 (SEQ ID NO: 23)

(SEQ ID NO: 23)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Dbu-NMeA-R-G-NH₂

Synthesis and purification methods: S01; P01
LCMS: A02
Rt: 28.6 min
m/3: 963.9

### Compound 24

### SEQ ID NO. 24

MW (calculated): 2470.95 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Dbu⁵,G⁸]NMU-8 (SEQ ID NO: 24)

(SEQ ID NO: 24)
C18DA-gGlu-gGlu-gGlu-Sar-Sar-Sar-Sar-Sar-Sar-Sar-
Sar-Sar-AEPipAc-Y-F-NMeL-Nle-Dbu-P-R-G-NH₂

Synthesis and purification methods: S01; P01
LCMS: A02
Rt: 29.4 min
m/3: 825.7

### Compound 25

### SEQ ID NO. 25

MW (calculated): 2740.25 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 25)

(SEQ ID NO: 25)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-R-P-R-G-
NH₂

Synthesis and purification methods: S01; P01
LCMS: A02
Rt: 28.9 min
m/3: 914.3

### Compound 26

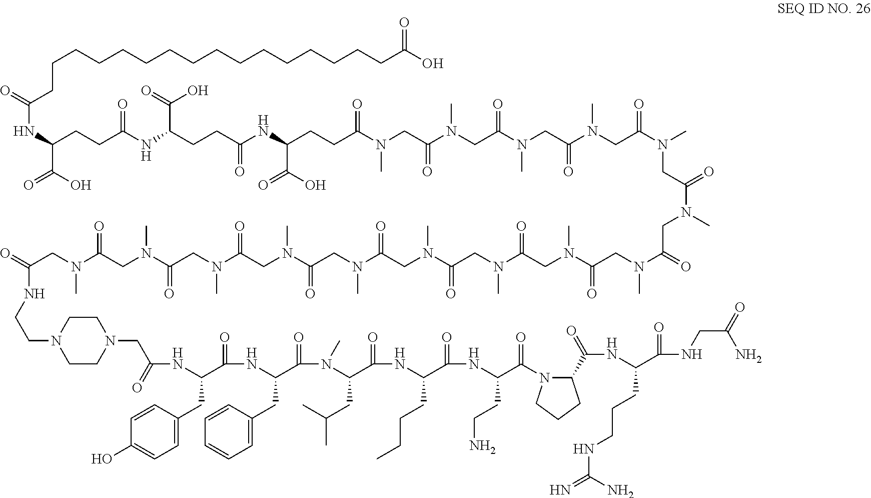 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCC(=O)O)C(=O)O)C(=O)O)C(=O)O)CC1)C(=O)N[C@@H](CCN)C(=O)N1CCC[C@H]1C(=O)N[C@@H](CCCNC(=N)N)C(=O)NCC(N)=O] [0522] MW (calculated): 2897.42 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Dbu⁵,G⁸]NMU-8 (SEQ ID NO: 26)

(SEQ ID NO: 26)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Dbu-P-R-G-NH₂

Synthesis and purification methods: S01; P01
LCMS: A02
Rt: 28.5 min
m/3: 966.6

### Compound 27

### SEQ ID NO. 27

MW (calculated): 2684.18 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,G⁸]NMU-8 (SEQ ID NO: 27)

(SEQ ID NO: 27)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-Dbu-P-R-
G-NH₂

Synthesis and purification methods: S01; P01
LCMS: A03
Rt: 29.9 min
m/2: 1342.8

### Compound 28

### SEQ ID NO. 28

MW (calculated): 2458.93 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Dbu⁵,NMeA⁶,G⁸]NMU-8 (SEQ ID NO: 28)

(SEQ ID NO: 28)
C18DA-gGlu-gGlu-gGlu-Sar-Sar-Sar-Sar-Sar-Sar-Sar-
Sar-Sar-AEPipAc-Y-F-NMeL-Nle-Dbu-NMeA-R-G-NH₂

Synthesis and purification methods: S01; P01
LCMS: A03
Rt: 30.3 min
m/2: 1230.1

### Compound 29

### SEQ ID NO. 29

MW (calculated): 2899.44 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Orn⁵,G⁶,G⁸]NMU-8 (SEQ ID NO: 29)

(SEQ ID NO: 29)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-G-R-G-NH₂

Synthesis and purification methods: S01; P01
LCMS: A03
Rt: 31.9 min
m/2: 1450.3

### Compound 30

### SEQ ID NO. 30

MW (calculated): 2913.46 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Orn⁵,bAla⁶,G⁸]NMU-8 (SEQ ID NO: 30)

(SEQ ID NO: 30)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-bAla-R-G-NH₂

Synthesis and purification methods: S01; P01
LCMS: A03
Rt: 31.9 min
m/2: 1457.3

### Compound 31

### SEQ ID NO. 31

MW (calculated): 2829.34 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,NMeA⁶,G⁸]NMU-8 (SEQ ID NO: 31)

(SEQ ID NO: 31)
C20DA-gGlu-gGlu-gGlu-gGlu-Sar-Sar-Sar-Sar-Sar-
Sar-Sar-Sar-Sar-Sar-Sar-Sar-AEPipAc-Y-F-NMeL-Nle-
Dbu-NMeA-R-G-NH₂

Synthesis and purification methods: S03; P01
LCMS: A01
Rt: 10.0 min
m/2: 1416.4

### Compound 32

### SEQ ID NO. 32

MW (calculated): 2869.41 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,K⁵,G⁸]NMU-8 (SEQ ID NO: 32)

(SEQ ID NO: 32)
C20DA-gGlu-gGlu-gGlu-gGlu-Sar-Sar-Sar-Sar-Sar-
Sar-Sar-Sar-Sar-Sar-Sar-Sar-AEPipAc-Y-F-NMeL-Nle-
K-P-R-G-NH₂

Synthesis and purification methods: S03; P01
LCMS: A01
Rt: 10.0 min
m/2: 1436.3

### Compound 33

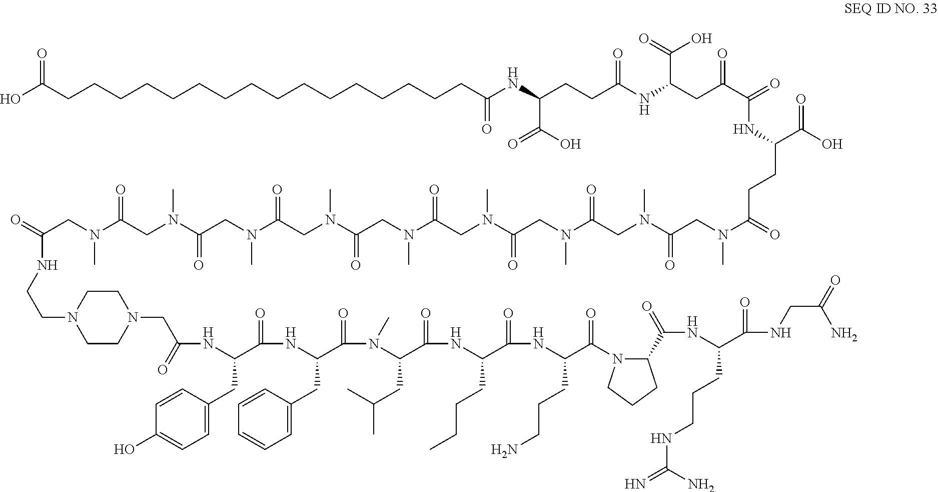 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)C(=O)C[C@H](NC(=O)CC[C@H](NC(=O)CCCCCCCCCCCCCCCCC(=O)O)C(=O)O)C(=O)O)C(=O)O)CC1)C(=O)N[C@@H](CCCN)C(=O)N1CCC[C@H]1C(=O)N[C@@H](CCCNC(=N)N)C(=O)NCC(N)=O] [0565] MW (calculated): 2484.97 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Orn⁵,G⁸]NMU-8 (SEQ ID NO: 33)

(SEQ ID NO: 33)
C18DA-gGlu-gGlu-gGlu-Sar-Sar-Sar-Sar-Sar-Sar-Sar-
Sar-Sar-AEPipAc-Y-F-NMeL-Nle-Orn-P-R-G-NH₂

Synthesis and purification methods: S01; P01
LCMS: A02
Rt: 29.0 min
m/3: 829.1

### Compound 34

### SEQ ID NO. 34

MW (calculated): 2927.49 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Orn⁵,NMeA⁶,G⁸]NMU-8 (SEQ ID NO: 34)

(SEQ ID NO: 34)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-NMeA-R-G-NH₂

Synthesis and purification methods: S01; P01
LCMS: A02
Rt: 32.3 min
m/3: 976.7

### Compound 35

### SEQ ID NO. 35

MW (calculated): 2754.28 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,NMeR⁵,G⁸]NMU-8 (SEQ ID NO: 35)

(SEQ ID NO: 35)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-NMeR-
P-R-G-NH₂

Synthesis and purification methods: S03; P01
LCMS: A01
Rt: 8.7 min
m/3: 918.9

### Compound 36

### SEQ ID NO. 36

MW (calculated): 2983.56 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,NMeR⁵,NMeA⁶,G⁸]NMU-8 (SEQ ID NO: 36)

(SEQ ID NO: 36)
C20DA-gGlu-gGlu-Glu-Sar-Sar-Sar-Sar-Sar-Sar-Sar-
Sar-Sar-Sar-Sar-Sar-Sar-Sar-Sar-AEPipAc-Y-F-NMeL-
Nle-NMeR-NMeA-R-G-NH₂

Synthesis and purification methods: S03; P01
LCMS: A01
Rt: 10.6 min
m/2: 1492.7

### Compound 37

### SEQ ID NO. 37

MW (calculated): 2742.27 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,NMeR⁵,NMeA⁶,G⁸]NMU-8 (SEQ ID NO: 37)

(SEQ ID NO: 37)
C18DA-gGlu-Glu-gGlu-Sar-Sar-Sar-Sar-Sar-Sar-Sar-
Sar-Sar-Sar-Sar-Sar-AEPipAc-Y-F-NMeL-Nle-NMeR-
NMeA-R-G-NH₂

Synthesis and purification methods: S03; P01
LCMS: A01
Rt: 8.7 min
m/3: 914.9

### Compound 38

### SEQ ID NO. 38

MW (calculated): 2672.17 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,NMeA⁶,G⁸]NMU-8 (SEQ ID NO: 38)

(SEQ ID NO: 38)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-Dbu-
NMeA-R-G-NH₂

Synthesis and purification methods: S01; P01
LCMS: A03
Rt: 29.8 min
m/2: 1336.8

### Compound 39

### SEQ ID NO. 39

MW (calculated): 2897.42 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 39)

(SEQ ID NO: 39)
C20DA-gGlu-glu-gGlu-gGlu-Sar-Sar-Sar-Sar-Sar-Sar-
Sar-Sar-Sar-Sar-Sar-Sar-AEPipAc-Y-F-NMeL-Nle-R-
P-R-G-NH₂

Synthesis and purification methods: S03; P01
LCMS: A01
Rt: 10.3 min
m/3: 968.0

### Compound 40

### SEQ ID NO. 40

MW (calculated): 2855.38 Da
2-[4-(2-(19-carboxynonadecanoyl-L-γ-glutamyl-L-γ-glutamyl-L-γ-glutamyl-L-γ-glutamyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,G⁸]NMU-8 (SEQ ID NO: 40)

(SEQ ID NO: 40)
C20DA-gGlu-gGlu-gGlu-gGlu-Sar-Sar-Sar-Sar-Sar-
Sar-Sar-Sar-Sar-Sar-Sar-Sar-AEPipAc-Y-F-NMeL-Nle-
Orn-P-R-G-NH₂

Synthesis and purification methods: S03; P01
LCMS: A01
Rt: 10.0 min
m/3: 952.7

### Compound 41

### SEQ ID NO. 41

MW (calculated): 2813.3 Da
2-[4-(2-(17-carboxyheptadecanoyl-L-γ-glutamyl-L-γ-glutamyl-L-γ-glutamyl-L-γ-glutamyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,G⁸]NMU-8 (SEQ ID NO: 41)

(SEQ ID NO: 41)
C18DA-gGlu-gGlu-gGlu-gGlu-Sar-Sar-Sar-Sar-Sar-
Sar-Sar-Sar-Sar-Sar-Sar-Sar-AEPipAc-Y-F-NMeL-Nle-
Dbu-P-R-G-NH₂

Synthesis and purification methods: S03; P01
LCMS: A01
Rt: 7.9 min
m/3: 938.6

### Compound 42

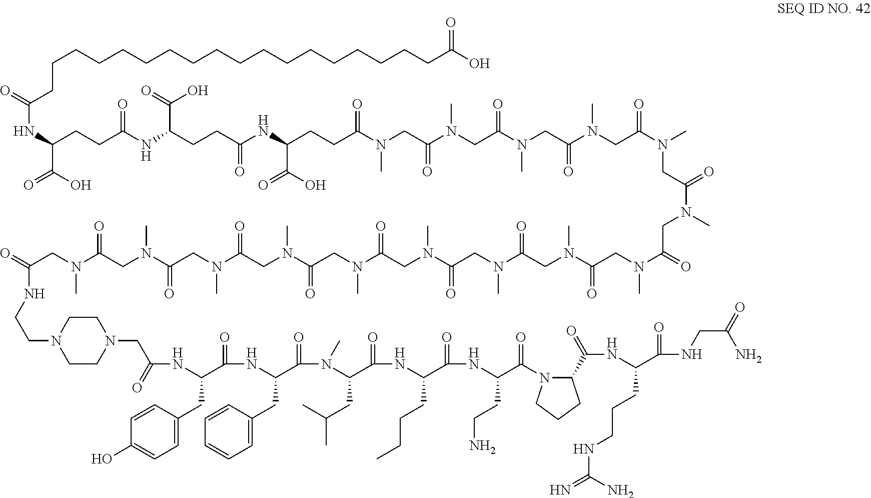 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCCCC(=O)O)C(=O)O)C(=O)O)C(=O)O)CC1)C(=O)N[C@@H](CCN)C(=O)N1CCC[C@H]1C(=O)N[C@@H](CCCNC(=N)N)C(=O)NCC(N)=O] [0620] MW (calculated): 2925.47 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Dbu⁵,G⁸]NMU-8 (SEQ ID NO: 42)

(SEQ ID NO: 42)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Dbu-P-R-G-NH₂

Synthesis and purification methods: S01; P01
LCMS: A03
Rt: 32.8 min
m/3: 975.9

### Compound 43

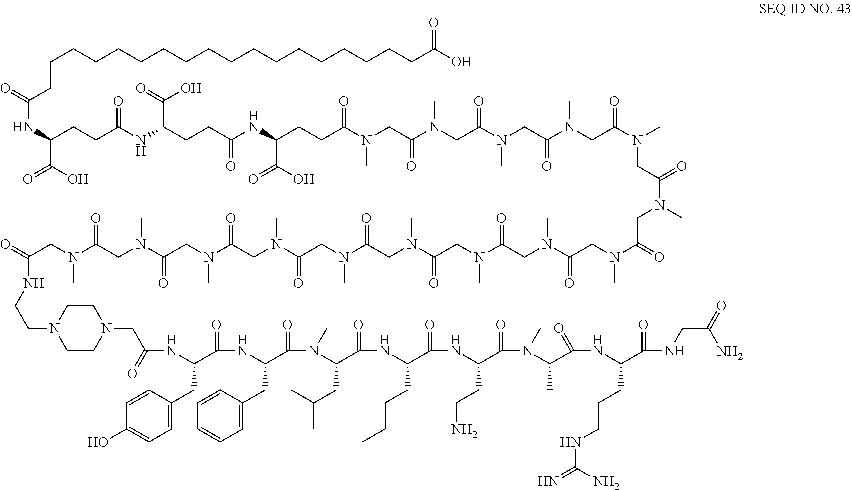 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCCCC(=O)O)C(=O)O)C(=O)O)C(=O)O)CC1)C(=O)N[C@@H](CCN)C(=O)N(C)[C@@H](C)C(=O)N[C@@H](CCCNC(=N)N)C(=O)NCC(N)=O] [0627] MW (calculated): 2913.46 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,Dbu⁵,NMeA⁶,G⁸]NMU-8 (SEQ ID NO: 43)

(SEQ ID NO: 43)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Dbu-NMeA-R-G-NH₂

Synthesis and purification methods: S01; P01
LCMS: A03
Rt: 32.7 min
m/2: 1457.4

### Compound 44

### SEQ ID NO. 44

MW (calculated): 2343.85 Da
2-[4-(2-(17-carboxyheptadecanoyl-L-γ-glutamyl-L-γ-glutamyl-N-methylglycyl-N-methylglycyl-N-methylglycyl-N-methylglycyl-N-methylglycyl-N-methylglycyl-N-methylglycyl-N-methylglycyl- N-methylglycylamino)ethyl)piper-azin-1-yl]acetyl-[NMeL³,Nle⁴,Orn⁵,NMeA⁶,G⁸]NMU-8 (SEQ ID NO: 44)

(SEQ ID NO: 44)
C18DA-gGlu-gGlu-Sar-Sar-Sar-Sar-Sar-Sar-Sar-Sar-
Sar-AEPipAc-Y-F-NMeL-Nle-Orn-NMeA-R-G-NH₂

Synthesis and purification methods: S03; P01
LCMS: A01
Rt: 8.9 min
m/3: 782.3

### Compound 45

### SEQ ID NO. 45

MW (calculated): 2945.46 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,4Pal⁵,G⁸]NMU-8 (SEQ ID NO: 45)

(SEQ ID NO: 45)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-4Pal-P-R-G-NH₂

Synthesis and purification methods: S03; P01
LCMS: A02
Rt: 28.3 min
m/3: 982.9

### Compound 46

### SEQ ID NO. 46

MW (calculated): 2925.47 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,K⁵,G⁸]NMU-8 (SEQ ID NO: 46)

(SEQ ID NO: 46)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-K-P-R-G-NH₂

Synthesis and purification methods: S01; P01
LCMS: A02
Rt: 28.4 min
m/1: 2925.4

### Compound 47

### SEQ ID NO. 47

MW (calculated): 2913.46 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,K⁵,NMeA⁶,G⁸]NMU-8 (SEQ ID NO: 47)

(SEQ ID NO: 47)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-K-NMeA-R-G-NH₂

Synthesis and purification methods: S01; P01
LCMS: A02
Rt: 28.3 min
m/3: 972.1

### Compound 48

### SEQ ID NO. 48

MW (calculated): 2933.45 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,2Pal⁵,NMeA⁶,G⁸]NMU-8 (SEQ ID NO: 48)

(SEQ ID NO: 48)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-2Pal-NMeA-R-G-NH₂

Synthesis and purification methods: S01; P01
LCMS: A02
Rt: 31.7 min
m/3: 978.3

### Compound 49

### SEQ ID NO. 49

MW (calculated): 2953.53 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,K⁶,G⁸]NMU-8 (SEQ ID NO: 49)

(SEQ ID NO: 49)
C20DA-gGlu-Glu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-K-P-R-G-NH₂

Synthesis and purification methods: S01; P01
LCMS: A02
Rt: 31.6 min
m/3: 985.6

### Compound 50

### SEQ ID NO. 50

MW (calculated): 2941.52 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,K⁵,NMeA⁶,G⁸]NMU-8 (SEQ ID NO: 50)

(SEQ ID NO: 50)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-K-NMeA-R-G-NH₂

Synthesis and purification methods: S01; P01
LCMS: A02
Rt: 32.3 min
m/3: 981.4

### Compound 51

### SEQ ID NO. 51

MW (calculated): 2934.44 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,G⁸]NMU-8 (SEQ ID NO: 51)

(SEQ ID NO: 51)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-H-P-R-G-NH₂

Synthesis and purification methods: S01; P01
LCMS: A02
Rt: 28.8 min
m/3: 979.1

### Compound 52

### SEQ ID NO. 52

MW (calculated): 2967.51 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,NMeR⁵,G⁸]NMU-8 (SEQ ID NO: 52)

(SEQ ID NO: 52)
C18DA-gGlu-glu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-NMeR-P-R-G-NH₂

Synthesis and purification methods: S03; P01
LCMS: A02
Rt: 28.8 min
m/3: 990.3

### Compound 53

### SEQ ID NO. 53

MW (calculated): 2955.5 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,NMeR⁵,NMeA⁶,G⁸]NMU-8 (SEQ ID NO: 53)

(SEQ ID NO: 53)
C18DA-gGlu-Glu-Glu-Sar-Sar-Sar-Sar-Sar-Sar-Sar-
Sar-Sar-Sar-Sar-Sar-Sar-Sar-Sar-AEPipAc-Y-F-NMeL-
Nle-NMeR-NMeA-R-G-NH₂

Synthesis and purification methods: S03; P01
LCMS: A02
Rt: 28.8 min
m/3: 986.4

### Compound 54

### SEQ ID NO. 54

MW (calculated): 2945.46 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,3Pal⁵,G⁸]NMU-8 (SEQ ID NO: 54)

(SEQ ID NO: 54)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-3Pal-P-R-G-NH₂

Synthesis and purification methods: S03; P01
LCMS: A02
Rt: 28.5 min
m/3: 982.9

### Compound 55

### SEQ ID NO. 55

MW (calculated): 2922.43 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,H⁵,NMeA⁶,G⁸]NMU-8 (SEQ ID NO: 55)

(SEQ ID NO: 55)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-H-NMeA-R-G-NH₂

Synthesis and purification methods: S03; P01
LCMS: A02
Rt: 28.4 min
m/3: 975.3

### Compound 56

### SEQ ID NO. 56

MW (calculated): 2945.46 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,2Pal⁵,G⁸]NMU-8 (SEQ ID NO: 56)

(SEQ ID NO: 56)
C18DA-gGlu-gGlu-Glu-Sar-Sar-Sar-Sar-Sar-Sar-Sar-
Sar-Sar-Sar-Sar-Sar-Sar-Sar-Sar-AEPipAc-Y-F-NMeL-
Nle-2Pal-P-R-G-NH₂

Synthesis and purification methods: S03; P01
LCMS: A02
Rt: 28.8 min
m/3: 982.9

### Compound 57

### SEQ ID NO. 57

MW (calculated): 2933.45 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,3Pal⁵,NMeA⁶,G⁸]NMU-8 (SEQ ID NO: 57)

(SEQ ID NO: 57)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-3Pal-NMeA-R-G-NH₂

Synthesis and purification methods: S03; P01
LCMS: A02
Rt: 28.4 min
m/3: 979.1

### Compound 58

### SEQ ID NO. 58

MW (calculated): 2933.45 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[NMeL³,Nle⁴,4Pal⁵,NMeA⁶,G⁸]NMU-8 (SEQ ID NO: 58)

(SEQ ID NO: 58)
C18DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-4Pal-NMeA-R-G-NH₂

Synthesis and purification methods: S03; P01
LCMS: A02
Rt: 28.3 min
m/3: 978.6

### Compound 59

### SEQ ID NO. 59

MW (calculated): 2700.23 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Dbu⁵,NMeA⁶,G⁸]NMU-8 (SEQ ID NO: 59)

(SEQ ID NO: 59)
C20DA-gGlu-gGlu-gGlu-Sar-Sar-Sar-Sar-Sar-Sar-Sar-
Sar-Sar-Sar-Sar-Sar-AEPipAc-Y-F-NMeL-Nle-Dbu-
NMeA-R-G-NH₂

Synthesis and purification methods: S03; P01
LCMS: A01
Rt: 10.5 min
m/3: 901.1

### Compound 60

### SEQ ID NO. 60

MW (calculated): 2499.00 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Dbu⁵,G⁸]NMU-8 (SEQ ID NO: 60)

(SEQ ID NO: 60)
C20DA-gGlu-gGlu-gGlu-Sar-Sar-Sar-Sar-Sar-Sar-
Sar-Sar-Sar-AEPipAc-Y-F-NMeL-Nle-Dbu-P-R-G-NH₂

Synthesis and purification methods: S03; P01
LCMS: A01
Rt: 10.9 min
m/3: 833.9

### Compound 61

### SEQ ID NO. 61

MW (calculated): 2486.99 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Dbu⁵,NMeA⁶,G⁸]NMU-8 (SEQ ID NO: 61)

(SEQ ID NO: 61)
C20DA-gGlu-gGlu-gGlu-Sar-Sar-Sar-Sar-Sar-Sar-Sar-
Sar-Sar-AEPipAc-Y-F-NMeL-Nle-Dbu-NMeA-R-G-NH₂

Synthesis and purification methods: S03; P01
LCMS: A01
Rt: 10.9 min
m/3: 829.9

### Compound 62

### SEQ ID NO. 62

MW (calculated): 2543.06 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,NMeA⁶,G⁸]NMU-8 (SEQ ID NO: 62)

(SEQ ID NO: 62)
C20DA-gGlu-gGlu-gGlu-Sar-Sar-Sar-Sar-Sar-Sar-Sar-
Sar-Sar-AEPipAc-Y-F-NMeL-Nle-R-NMeA-R-G-NH₂

Synthesis and purification methods: S03; P01
LCMS: A01
Rt: 11.1 min
m/3: 848.6

### Compound 63

### SEQ ID NO. 63

MW (calculated): 2756.29 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,NMeA⁶,G⁸]NMU-8 (SEQ ID NO: 63)

(SEQ ID NO: 63)
C20DA-gGlu-gGlu-gGlu-Sar-Sar-Sar-Sar-Sar-Sar-Sar-
Sar-Sar-Sar-Sar-Sar-AEPipAc-Y-F-NMeL-Nle-R-
NMeA-R-G-NH₂

Synthesis and purification methods: S03; P01
LCMS: A01
Rt: 10.7 min
m/3: 919.8

### Compound 65

### SEQ ID NO. 65

MW (calculated): 2555.07 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,G⁸]NMU-8 (SEQ ID NO: 65)

(SEQ ID NO: 65)
C20DA-gGlu-gGlu-Glu-Sar-Sar-Sar-Sar-Sar-Sar-Sar-
Sar-Sar-AEPipAc-Y-F-NMeL-Nle-R-P-R-G-NH₂

Synthesis and purification methods: S02; P02
LCMS: A03
Rt: 33.9 min
m/2: 1278.2

### Compound 66

### SEQ ID NO. 66

MW (calculated): 2385.89 Da
2-[4-(2-(17-carboxyheptadecanoyl-L-γ-glutamyl-L-γ-glutamyl-N-methylglycyl-N-methylglycyl-N-methylglycyl-N-methylglycyl-N-methylglycyl-N-methylglycyl-N-methylglycyl-N-methylglycyl- N-methylglycylamino)ethyl)piper-azin-1-yl]acetyl-[NMeL³,Nle⁴,NMeA⁶,G⁸]NMU-8 (SEQ ID NO: 66)

(SEQ ID NO: 66)
C18DA-gGlu-gGlu-Sar-Sar-Sar-Sar-Sar-Sar-Sar-Sar-
Sar-AEPipAc-Y-F-NMeL-Nle-R-NMeA-R-G-NH₂

Synthesis and purification methods: S03; P01
LCMS: A01
Rt: 9.2 min
m/3: 796.3

### Compound 67

### SEQ ID NO. 67

MW (calculated): 2397.90 Da
2-[4-(2-(17-carboxyheptadecanoyl-L-γ-glutamyl-L-γ-glutamyl-N-methylglycyl-N-methylglycyl-N-methylglycyl-N-methylglycyl-N-methylglycyl-N-methylglycyl-N-methylglycyl-N-methylglycyl- N-methylglycylamino)ethyl)piper-azin-1-yl]acetyl-[NMeL³,Nle⁴,G⁸]NMU-8 (SEQ ID NO: 67)

(SEQ ID NO: 67)
C18DA-gGlu-gGlu-Sar-Sar-Sar-Sar-Sar-Sar-Sar-Sar-
Sar-AEPipAc-Y-F-NMeL-Nle-R-P-R-G-NH₂

Synthesis and purification methods: S01; P01
LCMS: A02
Rt: 30.0 min
m/3: 800.1

### Compound 68

### SEQ ID NO. 68

MW (calculated): 2313.78 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N- methylglycylamino)ethyl)piperazin-1-yl]acetyl-[NMeL³,Nle⁴,G⁸]NMU-8 (SEQ ID NO: 68)

(SEQ ID NO: 68)
C18DA-gGlu-gGlu-gGlu-Sar-Sar-Sar-Sar-Sar-Sar-
AEPipAc-Y-F-NMeL-Nle-R-P-R-G-NH₂

Synthesis and purification methods: S01; P01
LCMS: A02
Rt: 29.8 min
m/3: 772.2

### Compound 69

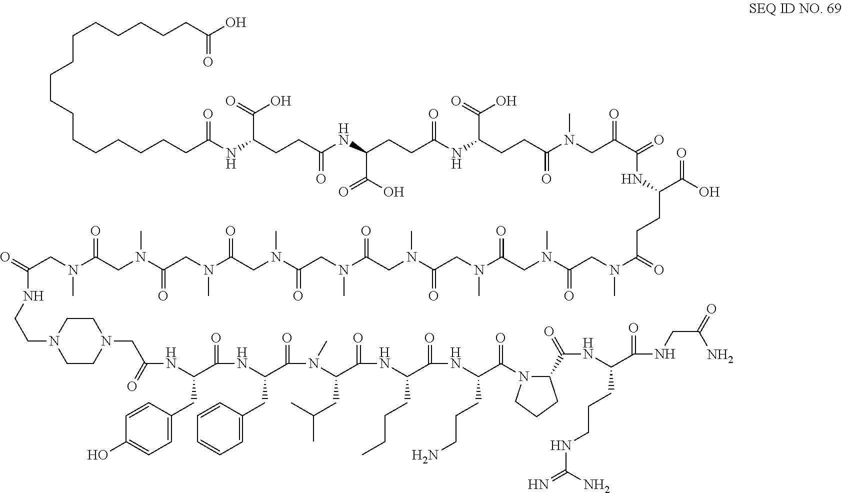 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)C(=O)CN(C)C(=O)CC[C@H](NC(=O)CC[C@H](NC(=O)CC[C@H](NC(=O)CCCCCCCCCCCCCCCCC(=O)O)C(=O)O)C(=O)O)C(=O)O)C(=O)O)CC1)C(=O)N[C@@H](CCCN)C(=O)N1CCC[C@H]1C(=O)N[C@@H](CCCNC(=N)N)C(=O)NCC(N)=O] [0778] MW (calculated): 2698.21 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,G⁸]NMU-8 (SEQ ID NO: 69)

(SEQ ID NO: 69)
C18DA-gGlu-gGlu-gGlu-Sar-Sar-Sar-Sar-Sar-Sar-Sar-
Sar-Sar-Sar-Sar-Sar-AEPipAc-Y-F-NMeL-Nle-Orn-P-R-
G-NH₂

Synthesis and purification methods: S01; P01
LCMS: A03
Rt: 29.7 min
m/2: 1349.8

### Compound 70

### SEQ ID NO. 70

MW (calculated): 2472.96 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,Orn⁵,NMeA⁶,G⁸]NMU-8 (SEQ ID NO: 70)

(SEQ ID NO: 70)
C18DA-gGlu-gGlu-gGlu-Sar-Sar-Sar-Sar-Sar-Sar-Sar-
Sar-Sar-AEPipAc-Y-F-NMeL-Nle-Orn-NMeA-R-G-NH₂

Synthesis and purification methods: S01; P01
LCMS: A03
Rt: 30.1 min
m/2: 1237.2

### Compound 71

### SEQ ID NO. 71

MW (calculated): 2184.66 Da
2-[4-(2-(17-carboxyheptadecanoyl-L-γ-glutamyl-L-γ-glutamyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 71)

(SEQ ID NO: 71)
C18DA-gGlu-gGlu-Sar-Sar-Sar-Sar-Sar-Sar-AEPipAc-
Y-F-NMeL-Nle-R-P-R-G-NH₂

Synthesis an purification methods: S01; P01
LCMS: A03
Rt: 31.8 min
m/3: 729.0

### Compound 72

### SEQ ID NO. 72

MW (calculated): 2527.01 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,G⁸]NMU-8 (SEQ ID NO: 72)

(SEQ ID NO: 72)
C18DA-gGlu-gGlu-gGlu-Sar-Sar-Sar-Sar-Sar-Sar-Sar-
Sar-Sar-AEPipAc-Y-F-NMeL-Nle-R-P-R-G-NH₂

Synthesis and purification methods: S01; P01
LCMS: A03
Rt: 30.7 min
m/3: 843.2

### Compound 73

### SEQ ID NO. 73

MW (calculated): 2515.00 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,NMeA⁶,G⁸]NMU-8 (SEQ ID NO: 73)

(SEQ ID NO: 73)
C18DA-gGlu-gGlu-gGlu-Sar-Sar-Sar-Sar-Sar-Sar-Sar-
Sar-Sar-AEPipAc-Y-F-NMeL-Nle-R-NMeA-R-G-NH₂

Synthesis and purification methods: S03; P01
LCMS: A01
Rt: 8.8 min
m/3: 839.6

### Compound 74

### SEQ ID NO. 74

MW (calculated): 2513.03 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyla-mino)ethyl)piperazin-1-yl]acetyl-[NMeL³,Nle⁴,hLys⁵,G⁸]NMU-8 (SEQ ID NO: 74)

(SEQ ID NO: 74)
C18DA-gGlu-gGlu-gGlu-Sar-Sar-Sar-Sar-Sar-Sar-Sar-
Sar-Sar-AEPipAc-Y-F-NMeL-Nle-hLys-P-R-G-NH₂

Synthesis and purification methods: S03; P01
LCMS: A01
Rt: 8.7 min
m/3: 838.4

### Compound 75

### SEQ ID NO. 75

MW (calculated): 2910.42 Da
2-[4-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl)piperazin-1-yl]acetyl-[NMeL³,Nle⁴,G⁸]NMU-8 (SEQ ID NO: 75)

(SEQ ID NO: 75)
C18DA-gGlu-gGlu-Glu-Sar-Sar-Sar-Sar-Sar-Sar-Sar-
Sar-Sar-Sar-Sar-Sar-Sar-Sar-Sar-PipAc-Y-F-NMeL-
Nle-R-P-R-G-NH₂

Synthesis and purification methods: S01; P01
LCMS: A04
Rt: 29.9 min
m/2: 1455.3

### Compound 76

### SEQ ID NO. 76

MW (calculated): 2854.35 Da
2-[4-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl)piperazin-1-yl]acetyl-[NMeL³,Nle⁴,Dbu⁵,G⁸]NMU-8 (SEQ ID NO: 76)

(SEQ ID NO: 76)
C18DA-gGlu-gGlu-gGlu-Sar-Sar-Sar-Sar-Sar-Sar-Sar-
Sar-Sar-Sar-Sar-Sar-Sar-Sar-Sar-PipAc-Y-F-NMeL-
Nle-Dbu-P-R-G-NH₂

Synthesis and purification methods: S01; P01
LCMS: A04
Rt: 29.6 min
m/2: 1427.3

### Compound 77

### SEQ ID NO. 77

MW (calculated): 2697.18 Da
2-[4-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl)piperazin-1-yl]acetyl-[NMeL³,Nle⁴,G⁸]NMU-8 (SEQ ID NO: 77)

(SEQ ID NO: 77)
C18DA-gGlu-gGlu-gGlu-Sar-Sar-Sar-Sar-Sar-Sar-Sar-
Sar-Sar-Sar-Sar-Sar-PipAc-Y-F-NMeL-Nle-R-P-R-G-
NH₂

Synthesis and purification methods: S01; P01
LCMS: A04
Rt: 30.4 min
m/2: 1348.8

### Compound 78

### SEQ ID NO. 78

MW (calculated): 2725.24 Da
2-[4-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl)piperazin-1-yl]acetyl-[NMeL³,Nle⁴,G⁸]NMU-8 (SEQ ID NO: 78)

(SEQ ID NO: 78)
C20DA-gGlu-gGlu-Glu-Sar-Sar-Sar-Sar-Sar-Sar-Sar-
Sar-Sar-Sar-Sar-Sar-PipAc-Y-F-NMeL-Nle-R-P-R-G-
NH₂

Synthesis and purification methods: S01; P01
LCMS: A04
Rt: 33.5 min
m/2: 1362.8

### Compound 79

### SEQ ID NO. 79

MW (calculated): 2842.34 Da
2-[4-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl- N-methylglycyl-N-methylglycyl-N-methylglycyl-N-methylglycyl-N-methylglycyl-N-methylglycyl-N-methylglycyl-N-methylglycyl)piperazin-1-yl]acetyl-[NMeL³,Nle⁴,Dbu⁵,NMeA⁶,G⁸]NMU-8 (SEQ ID NO: 79)

(SEQ ID NO: 79)
C18DA-gGlu-gGlu-gGlu-Sar-Sar-Sar-Sar-Sar-Sar-Sar-
Sar-Sar-Sar-Sar-Sar-Sar-Sar-Sar-PipAc-Y-F-NMeL-
Nle-Dbu-NMeA-R-G-NH₂

Synthesis and purification methods: S01; P01
LCMS: A04
Rt: 29.6 min
m/2: 1421.3

### Compound 80

### SEQ ID NO. 80

MW (calculated): 2896.43 Da
2-[4-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl)piperazin-1-yl]acetyl-[NMeL³,Nle⁴,Orn⁵,G⁸]NMU-8 (SEQ ID NO: 80)

(SEQ ID NO: 80)
C20DA-gGlu-gGlu-gGlu-Sar-Sar-Sar-Sar-Sar-Sar-Sar-
Sar-Sar-Sar-Sar-Sar-Sar-Sar-Sar-PipAc-Y-F-NMeL-
Nle-Orn-P-R-G-NH₂

Synthesis and purification methods: S01; P01
LCMS: A04
Rt: 32.7 min
m/2: 1448.3

### Reference Compound Ref. 1

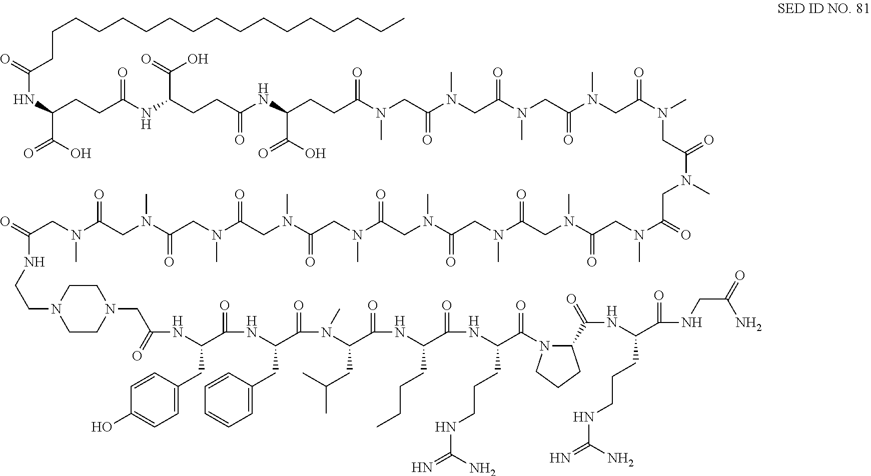 [SMILES: CCCCCCCCCCCCCCCCCC(=O)N[C@@H](CCC(=O)N[C@@H](CCC(=O)N[C@@H](CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)NCCN1CCN(CC(=O)N[C@@H](Cc2ccc(O)cc2)C(=O)N[C@@H](Cc2ccccc2)C(=O)N(C)[C@@H](CC(C)C)C(=O)N[C@@H](CCCC)C(=O)N[C@@H](CCCNC(=N)N)C(=O)N2CCC[C@H]2C(=O)N[C@@H](CCCNC(=N)N)C(=O)NCC(N)=O)CC1)C(=O)O)C(=O)O)C(=O)O] [0851] MW (calculated): 2923.51 Da
2-[4-(2-(stearoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycylamino)ethyl)piperazin-1-yl]acetyl-[NMeL³,Nle⁴,G⁸]NMU-8 (SEQ ID NO: 81)

(SEQ ID NO: 81)
C18A-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-R-P-R-G-NH₂

Synthesis and purification methods: S01; P01
LCMS: A04
Rt: 38.6 min
m/2: 1461.9

### Reference Compound Ref. 2

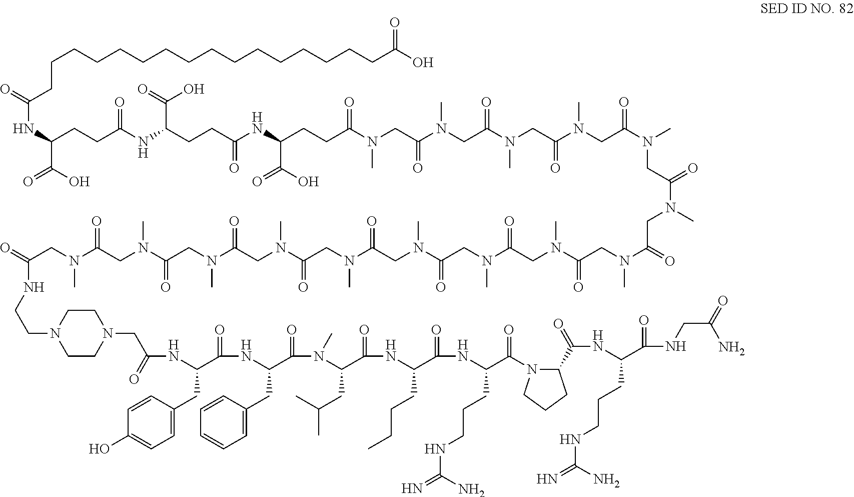 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCC(=O)O)C(=O)O)C(=O)O)C(=O)O)CC1)C(=O)N[C@@H](CCCNC(=N)N)C(=O)N1CCC[C@H]1C(=O)N[C@@H](CCCNC(=N)N)C(=O)NCC(N)=O] [0858] MW (calculated): 2939.46 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[Nle⁴,G⁸]NMU-8 (SEQ ID NO: 82)

(SEQ ID NO: 82)
C18DA-gGlu-gGlu-Glu-Sar-Sar-Sar-Sar-Sar-Sar-Sar-
Sar-Sar-Sar-Sar-Sar-Sar-Sar-Sar-AEPipAc-Y-F-L-
Nle-R-P-R-G-NH₂

Synthesis and purification methods: S01; P01
LCMS: A04
Rt: 32.2 min
m/2: 1469.9

### Reference Compound Ref. 3

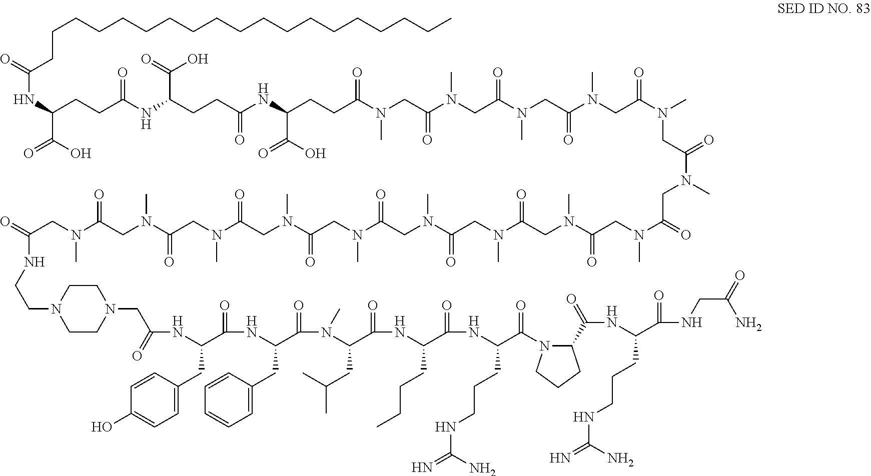 [SMILES: CCCCCCCCCCCCCCCCCCCC(=O)N[C@@H](CCC(=O)N[C@@H](CCC(=O)N[C@@H](CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)NCCN1CCN(CC(=O)N[C@@H](Cc2ccc(O)cc2)C(=O)N[C@@H](Cc2ccccc2)C(=O)N(C)[C@@H](CC(C)C)C(=O)N[C@@H](CCCC)C(=O)N[C@@H](CCCNC(=N)N)C(=O)N2CCC[C@H]2C(=O)N[C@@H](CCCNC(=N)N)C(=O)NCC(N)=O)CC1)C(=O)O)C(=O)O)C(=O)O] [0865] MW (calculated): 2909.52 Da
2-[4-(2-(icosanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl-N- methylglycyl-N-methyglycyl-N-methyglycyl-N-methylglycyl-N-methylglycyl-N-methylglycyl-N-methylglycyl-N-methylglycylamino)ethyl)piperazin-1-yl]acetyl-[NMeL³,Nle⁴,Orn⁵,G⁸]NMU-8 (SEQ ID NO: 83)

(SEQ ID NO: 83)
C20A-gGlu-gGlu-gGlu-Sar-Sar-Sar-Sar-Sar-Sar-Sar-
Sar-Sar-Sar-Sar-Sar-Sar-Sar-Sar-AEPipAc-Y-F-NMeL-
Nle-Orn-P-R-G-NH₂

Synthesis and purification methods: S01; P01
LCMS: A04
Rt: 43.9 min
m/2: 1454.9

### Reference Compound Ref. 4

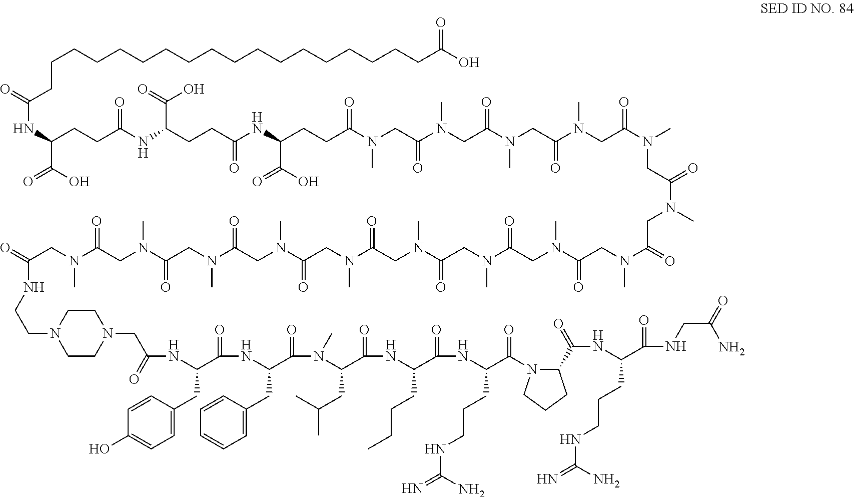 [SMILES: CCCC[C@H](NC(=O)[C@H](CC(C)C)N(C)C(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)CC[C@H](NC(=O)CC[C@H](NC(=O)CCCCCCCCCCCCCCCCCCC(=O)O)C(=O)O)C(=O)O)C(=O)O)CC1)C(=O)N[C@@H](CCCNC(=N)N)C(=O)N1CCC[C@H]1C(=O)N[C@@H](CCCNC(=N)N)C(=O)NCC(N)=O] [0872] MW (calculated): 2925.47 Da
2-[4-(2-(19-carboxynonadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycyl-N-methylglycyl-N-methylglycyl-N-methylglycylamino)ethyl)piper-azin-1-yl]acetyl-[Nle⁴,Orn⁵,G⁸]NMU-8 (SEQ ID NO: 84)

(SEQ ID NO: 84)
C20DA-gGlu-gGlu-gGlu-Sar-Sar-Sar-Sar-Sar-Sar-Sar-
Sar-Sar-Sar-Sar-Sar-Sar-Sar-Sar-AEPipAc-Y-F-L-
Nle-Orn-P-R-G-NH₂

Synthesis and purification methods: S01; P01
LCMS: A04
Rt: 35.4 min
m/2: 1462.9

### Reference Compound Ref. 5

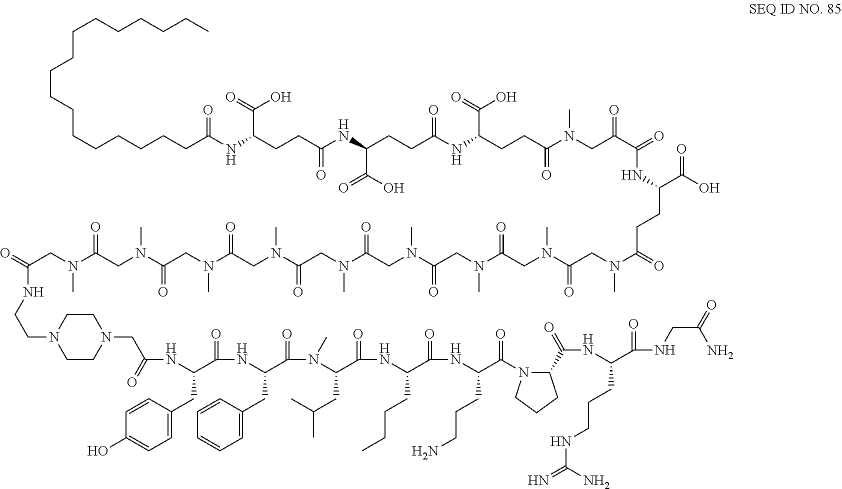 [SMILES: CCCCCCCCCCCCCCCCCC(=O)N[C@@H](CCC(=O)N[C@@H](CCC(=O)N[C@@H](CCC(=O)N(C)CC(=O)C(=O)N[C@@H](CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)NCCN1CCN(CC(=O)N[C@@H](Cc2ccc(O)cc2)C(=O)N[C@@H](Cc2ccccc2)C(=O)N(C)[C@@H](CC(C)C)C(=O)N[C@@H](CCCC)C(=O)N[C@@H](CCCN)C(=O)N2CCC[C@H]2C(=O)N[C@@H](CCCNC(=N)N)C(=O)NCC(N)=O)CC1)C(=O)O)C(=O)O)C(=O)O)C(=O)O] [0879] MW (calculated): 2668.23 Da
2-[4-(2-(stearoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycylamino)ethyl)piperazin-1-yl]acetyl-[NMeL³,Nle⁴,Orn⁵,G⁸]NMU-8 (SEQ ID NO: 85)

(SEQ ID NO: 85)
C18A-gGlu-gGlu-Glu-Sar-Sar-Sar-Sar-Sar-Sar-Sar-
Sar-Sar-Sar-Sar-Sar-AEPipAc-Y-F-NMeL-Nle-Orn-P-
R-G-NH₂

Synthesis and purification methods: S01; P01
LCMS: A04
Rt: 42.0 min
m/2: 1334.3

### Reference Compound Ref. 6

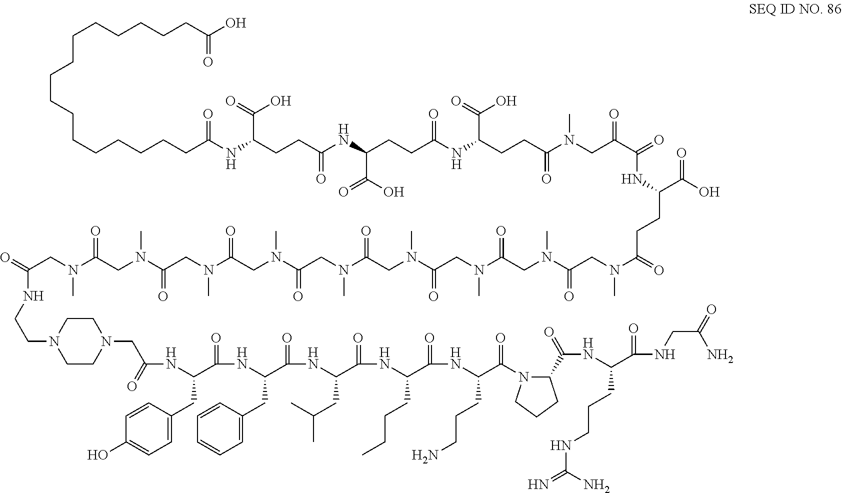 [SMILES: CCCC[C@H](NC(=O)[C@H](CC(C)C)NC(=O)[C@H](Cc1ccccc1)NC(=O)[C@H](Cc1ccc(O)cc1)NC(=O)CN1CCN(CCNC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CC[C@H](NC(=O)C(=O)CN(C)C(=O)CC[C@H](NC(=O)CC[C@H](NC(=O)CC[C@H](NC(=O)CCCCCCCCCCCCCCCCC(=O)O)C(=O)O)C(=O)O)C(=O)O)C(=O)O)CC1)C(=O)N[C@@H](CCCN)C(=O)N1CCC[C@H]1C(=O)N[C@@H](CCCNC(=N)N)C(=O)NCC(N)=O] [0886] MW (calculated): 2684.18 Da
2-[4-(2-(17-carboxyheptadecanoyl-L-γ-glutamyl-L-γ-glutamyl-L-γ-glutamyl-N-methylglycyl-N-methylglycyl-N-methylglycyl-N-methylglycyl-N-methylglycyl-N-methylglycyl-N- methylglycyl-N-methylglycyl-N-methylglycyl-N-methylglycyl-N-methylglycyl-N-methylglycylamino)ethyl)piperazin-1-yl]acetyl-[Nle⁴,Orn⁵,G⁸]NMU-8 (SEQ ID NO: 86)

(SEQ ID NO: 86)
C18DA-gGlu-gGlu-Glu-Sar-Sar-Sar-Sar-Sar-Sar-Sar-
Sar-Sar-Sar-Sar-Sar-AEPipAc-Y-F-L-Nle-Orn-P-R-G-
NH₂

Synthesis and purification methods: S01; P01
LCMS: A04
Rt: 29.7 min
m/2: 1342.3.

1. A compound of formula (I) 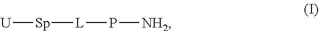
wherein
U is selected from the group Ug1 consisting of 15-carboxy-pentadecanoyl (C16DA), 17-carboxy-heptadecanoyl (C18DA), and 19-carboxy-nonadecanoyl (C20DA);
Sp is selected from the group Spg0 consisting of -(gGlu)ₙ-(Sar)ₘ- (SEQ ID NO: 149) wherein
n is selected from the group consisting of 1, 2, 3, and 4, and
m is selected from the group consisting of 3, 4, 5, 6, 7, 8, 9, 10, 11, 12, 13, 14, and 15, and
wherein a -gGlu- is linked to U and a -Sar- is linked to L;
L is selected from the group Lg1 consisting of 2-[4-(2-aminoethyl)piperazin-1-yl]acetic acid (AEPipAc), 2-(piperazin-1-yl)acetic acid (PipAc), and 2-[1-(2-aminoethyl)piperidin-4-yl]acetic acid (AECMP); and
P is a peptide having an amino acids sequence of formula (II)
(II)
(SEQ ID NO: 112)
Tyr-Phe-NMeLeu-Nle-X⁵-X⁶-Arg-Gly
wherein
X⁵ is selected from the group X⁵g1 consisting of Arg, Dbu, NMeArg, Orn, Lys, His, hLys, 2Pal, 3Pal, and 4Pal;
X⁶ is selected from the group X⁶g1 consisting of Pro, NMeAla, bAla, Gly.

2. The compound of formula (I) according to claim 1, wherein
U is selected from the group Ug1 consisting of 15-carboxy-pentadecanoyl (C16DA), 17-carboxy-heptadecanoyl (C18DA), and 19-carboxy-nonadecanoyl (C20DA);
Sp is selected from the group Spg1 consisting of -(gGlu)ₙ-(Sar)ₘ- (SEQ ID NO: 115) wherein
n is selected from the group consisting of 1, 2, 3, and 4, and
m is selected from the group consisting of 3, 6, 9, 12, and 15, and
wherein a -gGlu- is linked to U and a -Sar- is linked to L;
L is selected from the group Lg1 consisting of 2-[4-(2-aminoethyl)piperazin-1-yl]acetic acid (AEPipAc), 2-(piperazin-1-yl)acetic acid (PipAc), and 2-[I-(2-aminoethyl)piperidin-4-yl]acetic acid (AECMP); and
P is a peptide having an amino acids sequence of formula (II)
(II)
(SEQ ID NO: 112)
Tyr-Phe-NMeLeu-Nle-X⁵-X⁶-Arg-Gly
wherein
X⁵ is selected from the group X⁵g1 consisting of Arg, Dbu, NMeArg, Orn, Lys, His, hLys, 2Pal, 3Pal, and 4Pal;
X⁶ is selected from the group X⁶g1 consisting of Pro, NMeAla, bAla, Gly.

3. The compound of formula (I) according to claim 1, wherein
U is selected from the group Ug2 consisting of 17-carboxy-heptadecanoyl (C18DA), and 19-carboxy-nonadecanoyl (C20DA).

4. The compound of formula (I) according to claim 1, wherein
Sp is selected from the group Spg2 consisting of -(gGlu)ₙ-(Sar)ₘ- (SEQ ID NO: 116), wherein
n is selected from the group consisting of 3 and 4, and
m is selected from the group consisting of 9, 12 and 15.

5. The compound of formula (I) according to claim 1, wherein
L is selected from the group Lg2 consisting of 2-[4-(2-aminoethyl)piperazin-1-yl]acetic acid (AEPipAc), and 2-(piperazin-1-yl)acetic acid (PipAc).

6. The compound of formula (I) according to claim 1, wherein
X⁵ is selected from the group X⁵g2 consisting of Arg, Dbu, NMeArg, Orn and Lys; and
X⁶ is selected from the group X⁶g2 consisting of Pro, and NMeAla.

7. The compound of formula (I) according to claim 1, wherein
U is selected from the group Ug2 consisting of C18DA and C20DA;
Sp is selected from the group Spg2 consisting of -(gGlu)ₙ-(Sar)ₘ- (SEQ ID NO: 116), wherein
n is selected from the group consisting of 3, and 4, and
m is selected from the group consisting of 9, 12 and 15;
L is selected from the group Lg2 consisting of 2-[4-(2-aminoethyl)piperazin-1-yl]acetic acid (AEPipAc), and 2-(piperazin-1-yl)acetic acid (PipAc); and
P is the peptide of formula (II), wherein P is selected from the group consisting of:
(SEQ ID NO: 91)
Tyr-Phe-NMeLeu-Nle-Arg-Pro-Arg-Gly;
(SEQ ID NO: 92)
Tyr-Phe-NMeLeu-Nle-Orn-Pro-Arg-Gly;
(SEQ ID NO: 93)
Tyr-Phe-NMeLeu-Nle-Dbu-Pro-Arg-Gly;
(SEQ ID NO: 94)
Tyr-Phe-NMeLeu-Nle-Arg-NMeAla-Arg-Gly;
(SEQ ID NO: 95)
Tyr-Phe-NMeLeu-Nle-Orn-NMeAla-Arg-Gly;
(SEQ ID NO: 96)
Tyr-Phe-NMeLeu-Nle-Dbu-NMeAla-Arg-Gly;
(SEQ ID NO: 97)
Tyr-Phe-NMeLeu-Nle-Lys-Pro-Arg-Gly;
(SEQ ID NO: 98)
Tyr-Phe-NMeLeu-Nle-Lys-NMeAla-Arg-Gly;
(SEQ ID NO: 99)
Tyr-Phe-NMeLeu-Nle-Orn-Gly-Arg-Gly;
(SEQ ID NO: 100)
Tyr-Phe-NMeLeu-Nle-Orn-bAla-Arg-Gly;
(SEQ ID NO: 101)
Tyr-Phe-NMeLeu-Nle-NMeArg-NMeAla-Arg-Gly;
(SEQ ID NO: 102)
Tyr-Phe-NMeLeu-Nle-4Pal-Pro-Arg-Gly;
(SEQ ID NO: 103)
Tyr-Phe-NMeLeu-Nle-2Pal-NMeAla-Arg-Gly;
(SEQ ID NO: 104)
Tyr-Phe-NMeLeu-Nle-His-Pro-Arg-Gly;
(SEQ ID NO: 105)
Tyr-Phe-NMeLeu-Nle-NMeArg-Pro-Arg-Gly;
(SEQ ID NO: 106)
Tyr-Phe-NMeLeu-Nle-3Pal-Pro-Arg-Gly;
(SEQ ID NO: 107)
Tyr-Phe-NMeLeu-Nle-His-NMeAla-Arg-Gly;
(SEQ ID NO: 108)
Tyr-Phe-NMeLeu-Nle-2Pal-Pro-Arg-Gly-;
(SEQ ID NO: 109)
Tyr-Phe-NMeLeu-Nle-3Pal-NMeAla-Arg-Gly;
(SEQ ID NO: 110)
Tyr-Phe-NMeLeu-Nle-4Pal-NMeAla-Arg-Gly;
and
(SEQ ID NO: 111)
Tyr-Phe-NMeLeu-Nle-hLys-Pro-Arg-Gly.

8. The compound of formula (I) according to claim 1, wherein the compound is selected from the group consisting of:
(SEQ ID NO: 1)
C20DA-gGlu₃-Sar₁₅-AEPipAc-Tyr-Phe-NMeLeu-Nle-Orn-Pro-Arg-Gly-NH₂;
(SEQ ID NO: 2)
C18DA-gGlu₄-Sar₁₂-AEPipAc-Tyr-Phe-NMeLeu-Nle-Arg-Pro-Arg-Gly-NH₂;
(SEQ ID NO: 3)
C20DA-gGlu₄-Sar₁₅-AEPipAc-Tyr-Phe-NmeLeu-Nle-Orn-Pro-Arg-Gly-NH₂;
(SEQ ID NO: 4)
C20DA-Glu₄-Sar₁₅-AEPipAc-Tyr-Phe-NMeLeu-Nle-Dbu-Pro-Arg-Gly-NH₂;
(SEQ ID NO: 5)
C18DA-gGlu₄-Sar₁₂-AEPipAc-Tyr-Phe-NMeLeu-Nle-Orn-Pro-Arg-Gly-NH₂;
(SEQ ID NO: 6)
C20DA-gGlu₃-Sar₁₂-AEPipAc-Tyr-Phe-NMeLeu-Nle-Arg-Pro-Arg-Gly-NH₂;
(SEQ ID NO: 7)
C18DA-gGlu₃-Sar₁₅-AEPipAc-Tyr-Phe-NMeLeu-Nle-Arg-Pro-Arg-Gly-NH₂;
(SEQ ID NO: 8)
C20DA-gGlu₃-Sar₁₂-AEPipAc-Tyr-Phe-NMeLeu-Nle-Dbu-Pro-Arg-Gly-NH₂;
(SEQ ID NO: 9)
C20DA-gGlu₃-Sar₁₅-AEPipAc-Tyr-Phe-NMeLeu-Nle-Arg-Pro-Arg-Gly-NH₂;
(SEQ ID NO: 10)
C20DA-gGlu₃-Sar₁₅-AEPipAc-Tyr-Phe-NMeLeu-Nle-Arg-NmeAla-Arg-Gly-NH₂;
(SEQ ID NO: 11)
C18DA-gGlu₃-Sar₁₅-AEPipAc-Tyr-Phe-NMeLeu-Nle-Arg-NmeAla-Arg-Gly-NH₂;
(SEQ ID NO: 12)
C18DA-gGlu₃-Sar₁₂-AEPipAc-Tyr-Phe-NMeLeu-Nle-Arg-NmeAla-Arg-Gly-NH₂;
(SEQ ID NO: 13)
C20DA-gGlu₄-Sar₁₂-AEPipAc-Tyr-Phe-NMeLeu-Nle-Orn-NmeAla-Arg-Gly-NH₂;
(SEQ ID NO: 14)
C20DA-gGlu₄-Sar₁₅-AEPipAc-Tyr-Phe-NMeLeu-Nle-Orn-NmeAla-Arg-Gly-NH₂;
(SEQ ID NO: 15)
C20DA-gGlu₄-Sar₁₂-AEPipAc-Tyr-Phe-NMeLeu-Nle-Dbu-Pro-Arg-Gly-NH₂;
(SEQ ID NO: 16)
C20DA-gGlu₄-Sar₁₅-AEPipAc-Tyr-Phe-NMeLeu-Nle-Dbu-NmeAla-Arg-Gly-NH₂;
(SEQ ID NO: 17)
C20DA-gGlu₄-Sar₁₅-AEPipAc-Tyr-Phe-NMeLeu-Nle-Lys-Pro-Arg-Gly-NH₂;
(SEQ ID NO: 18)
C20DA-gGlu₄-Sar₁₅-AEPipAc-Tyr-Phe-NMeLeu-Nle-Lys-NmeAla-Arg-Gly-NH₂;
(SEQ ID NO: 19)
C20DA-gGlu₄-Sar₁₂-AEPipAc-Tyr-Phe-NMeLeu-Nle-Lys-NmeAla-Arg-Gly-NH₂;
(SEQ ID NO: 20)
C18DA-gGlu₃-Sar₁₂-AEPipAc-Tyr-Phe-NMeLeu-Nle-Orn-NmeAla-Arg-Gly-NH₂;
(SEQ ID NO: 21)
C18DA-gGlu₃-Sar₁₅-AEPipAc-Tyr-Phe-NmeLeu-Nle-Orn-Pro-Arg-Gly-NH₂;
(SEQ ID NO: 22)
C18DA-gGlu₃-Sar₁₅-AEPipAc-Tyr-Phe-NMeLeu-Nle-Orn-NmeAla-Arg-Gly-NH₂;
(SEQ ID NO: 23)
C18DA-gGlu₃-Sar₁₅-AEPipAc-Tyr-Phe-NMeLeu-Nle-Dbu-NmeAla-Arg-Gly-NH₂;
(SEQ ID NO: 24)
C18DA-gGlu₃-Sar₉-AEPipAc-Tyr-Phe-NMeLeu-Nle-Dbu-Pro-Arg-Gly-NH₂;
(SEQ ID NO: 25)
C18DA-gGlu₃-Sar₁₂-AEPipAc-Tyr-Phe-NMeLeu-Nle-Arg-Pro-Arg-Gly-NH₂;
(SEQ ID NO: 26)
C18DA-gGlu₃-Sar₁₅-AEPipAc-Tyr-Phe-NMeLeu-Nle-Dbu-Pro-Arg-Gly-NH₂;
(SEQ ID NO: 27)
C18DA-gGlu₃-Sar₁₂-AEPipAc-Tyr-Phe-NMeLeu-Nle-Dbu-Pro-Arg-Gly-NH₂;
(SEQ ID NO: 28)
C18DA-gGlu₃-Sar₉-AEPipAc-Tyr-Phe-NMeLeu-Nle-Dbu-NmeAla-Arg-Gly-NH₂;
(SEQ ID NO: 29)
C20DA-gGlu₃-Sar₁₅-AEPipAc-Tyr-Phe-NMeLeu-Nle-Orn-Gly-Arg-Gly-NH₂;
(SEQ ID NO: 30)
C20DA-gGlu₃-Sar₁₅-AEPipAc-Tyr-Phe-NMeLeu-Nle-Orn-bAla-Arg-Gly-NH₂;
(SEQ ID NO: 31)
C20DA-gGlu₄-Sar₁₂-AEPipAc-Tyr-Phe-NMeLeu-Nle-Dbu-NmeAla-Arg-Gly-NH₂;
(SEQ ID NO: 32)
C20DA-gGlu₄-Sar₁₂-AEPipAc-Tyr-Phe-NMeLeu-Nle-Lys-Pro-Arg-Gly-NH₂;
(SEQ ID NO: 33)
C18DA-gGlu₃-Sar₉-AEPipAc-Tyr-Phe-NmeLeu-Nle-Orn-Pro-Arg-Gly-NH₂;
(SEQ ID NO: 34)
C20DA-gGlu₃-Sar₁₅-AEPipAc-Tyr-Phe-NMeLeu-Nle-Orn-NmeAla-Arg-Gly-NH₂;
(SEQ ID NO: 35)
C18DA-gGlu₃-Sar₁₂-AEPipAc-Tyr-Phe-NMeLeu-Nle-NmeArg-Pro-Arg-Gly-NH₂;
(SEQ ID NO: 36)
C20DA-gGlu₃-Sar₁₅-AEPipAc-Tyr-Phe-NMeLeu-Nle-NmeArg-NmeAla-Arg-Gly-NH₂;
(SEQ ID NO: 37)
C18DA-gGlu₃-Sar₁₂-AEPipAc-Tyr-Phe-NMeLeu-Nle-NmeArg-NmeAla-Arg-Gly-NH₂;
(SEQ ID NO: 38)
C18DA-gGlu3-Sar12-AEPipAc-Tyr-Phe-NMeLeu-Nle-Dbu-NmeAla-Arg-Gly-NH₂;
(SEQ ID NO: 39)
C20DA-gGlu₄-Sar₁₂-AEPipAc-Tyr-Phe-NMeLeu-Nle-Arg-Pro-Arg-Gly-NH₂;
(SEQ ID NO: 40)
C20DA-gGlu₄-Sar₁₂-AEPipAc-Tyr-Phe-NmeLeu-Nle-Orn-Pro-Arg-Gly-NH₂;
(SEQ ID NO: 41)
C18DA-gGlu₄-Sar₁₂-AEPipAc-Tyr-Phe-NMeLeu-Nle-Dbu-Pro-Arg-Gly-NH₂;
(SEQ ID NO: 42)
C20DA-gGlu₃-Sar₁₅-AEPipAc-Tyr-Phe-NMeLeu-Nle-Dbu-Pro-Arg-Gly-NH₂;
(SEQ ID NO: 43)
C20DA-gGlu₃-Sar₁₅-AEPipAc-Tyr-Phe-NMeLeu-Nle-Dbu-NmeAla-Arg-Gly-NH₂;
(SEQ ID NO: 44)
C18DA-gGlu₂-Sar₉-AEPipAc-Tyr-Phe-NMeLeu-Nle-Orn-NmeAla-Arg-Gly-NH₂;
(SEQ ID NO: 45)
C18DA-gGlu₃-Sar₁₅-AEPipAc-Tyr-Phe-NMeLeu-Nle-4Pal-Pro-Arg-Gly-NH₂;
(SEQ ID NO: 46)
C18DA-gGlu₃-Sar₁₅-AEPipAc-Tyr-Phe-NMeLeu-Nle-Lys-Pro-Arg-Gly-NH₂;
(SEQ ID NO: 47)
C18DA-gGlu₃-Sar₁₅-AEPipAc-Tyr-Phe-NMeLeu-Nle-Lys-NmeAla-Arg-Gly-NH₂;
(SEQ ID NO: 48)
C18DA-gGlu₃-Sar₁₅-AEPipAc-Tyr-Phe-NMeLeu-Nle-2Pal-NmeAla-Arg-Gly-NH₂;
(SEQ ID NO: 49)
C20DA-gGlu₃-Sar₁₅-AEPipAc-Tyr-Phe-NMeLeu-Nle-Lys-Pro-Arg-Gly-NH₂;
(SEQ ID NO: 50)
C20DA-gGlu₃-Sar₁₅-AEPipAc-Tyr-Phe-NMeLeu-Nle-Lys-NmeAla-Arg-Gly-NH₂;
(SEQ ID NO: 51)
C18DA-gGlu₃-Sar₁₅-AEPipAc-Tyr-Phe-NMeLeu-Nle-His-Pro-Arg-Gly-NH₂;
(SEQ ID NO: 52)
C18DA-gGlu₃-Sar₁₅-AEPipAc-Tyr-Phe-NMeLeu-Nle-NmeArg-Pro-Arg-Gly-NH₂;
(SEQ ID NO: 53)
C18DA-gGlu₃-Sar₁₅-AEPipAc-Tyr-Phe-NMeLeu-Nle-NmeArg-NmeAla-Arg-Gly-NH₂;
(SEQ ID NO: 54)
C18DA-gGlu₃-Sar₁₅-AEPipAc-Tyr-Phe-NMeLeu-Nle-3Pal-Pro-Arg-Gly-NH₂;
(SEQ ID NO: 55)
C18DA-gGlu₃-Sar₁₅-AEPipAc-Tyr-Phe-NMeLeu-Nle-His-NmeAla-Arg-Gly-NH₂;
(SEQ ID NO: 56)
C18DA-gGlu₃-Sar₁₅-AEPipAc-Tyr-Phe-NMeLeu-Nle-2Pal-Pro-Arg-Gly-NH2;
(SEQ ID NO: 57)
C18DA-gGlu₃-Sar₁₅-AEPipAc-Tyr-Phe-NMeLeu-Nle-3Pal-NmeAla-Arg-Gly-NH₂;
(SEQ ID NO: 58)
C18DA-gGlu₃-Sar₁₅-AEPipAc-Tyr-Phe-NMeLeu-Nle-4Pal-NmeAla-Arg-Gly-NH₂;
(SEQ ID NO: 59)
C20DA-gGlu₃-Sar₁₂-AEPipAc-Tyr-Phe-NMeLeu-Nle-Dbu-NmeAla-Arg-Gly-NH₂;
(SEQ ID NO: 60)
C20DA-gGlu₃-Sar₉-AEPipAc-Tyr-Phe-NMeLeu-Nle-Dbu-Pro-Arg-Gly-NH₂;
(SEQ ID NO: 61)
C20DA-gGlu₃-Sar₉-AEPipAc-Tyr-Phe-NMeLeu-Nle-Dbu-NmeAla-Arg-Gly-NH₂;
(SEQ ID NO: 62)
C20DA-gGlu₃-Sar₉-AEPipAc-Tyr-Phe-NMeLeu-Nle-Arg-NmeAla-Arg-Gly-NH₂;
(SEQ ID NO: 63)
C20DA-gGlu₃-Sar₁₂-AEPipAc-Tyr-Phe-NMeLeu-Nle-Arg-NmeAla-Arg-Gly-NH₂;
(SEQ ID NO: 65)
C20DA-gGlu₃-Sar₉-AEPipAc-Tyr-Phe-NMeLeu-Nle-Arg-Pro-Arg-Gly-NH₂;
(SEQ ID NO: 66)
C18DA-gGlu₂-Sar₉-AEPipAc-Tyr-Phe-NMeLeu-Nle-Arg-NmeAla-Arg-Gly-NH₂;
(SEQ ID NO: 67)
C18DA-gGlu₂-Sar₉-AEPipAc-Tyr-Phe-NMeLeu-Nle-Arg-Pro-Arg-Gly-NH₂;
(SEQ ID NO: 68)
C18DA-gGlu₃-Sar₆-AEPipAc-Tyr-Phe-NMeLeu-Nle-Arg-Pro-Arg-Gly-NH₂;
(SEQ ID NO: 69)
C18DA-gGlu₃-Sar₁₂-AEPipAc-Tyr-Phe-NmeLeu-Nle-Orn-Pro-Arg-Gly-NH₂;
(SEQ ID NO: 70)
C18DA-gGlu₃-Sar₉-AEPipAc-Tyr-Phe-NMeLeu-Nle-Orn-NmeAla-Arg-Gly-NH₂;
(SEQ ID NO: 71)
C18DA-gGlu₂-Sar₆-AEPipAc-Tyr-Phe-NMeLeu-Nle-Arg-Pro-Arg-Gly-NH₂;
(SEQ ID NO: 72)
C18DA-gGlu₃-Sar₉-AEPipAc-Tyr-Phe-NMeLeu-Nle-Arg-Pro-Arg-Gly-NH₂;
(SEQ ID NO: 73)
C18DA-gGlu₃-Sar₉-AEPipAc-Tyr-Phe-NMeLeu-Nle-Arg-NmeAla-Arg-Gly-NH₂;
(SEQ ID NO: 74)
C18DA-gGlu₃-Sar₉-AEPipAc-Tyr-Phe-NMeLeu-Nle-hLys-Pro-Arg-Gly-NH₂;
(SEQ ID NO: 75)
C18DA-gGlu₃-Sar₁₅-PipAc-Tyr-Phe-NMeLeu-Nle-Arg-Pro-Arg-Gly-NH₂;
(SEQ ID NO: 76)
C18DA-gGlu₃-Sar₁₅-PipAc-Tyr-Phe-NMeLeu-Nle-Dbu-Pro-Arg-Gly-NH₂;
(SEQ ID NO: 77)
C18DA-gGlu₃-Sar₁₂-PipAc-Tyr-Phe-NMeLeu-Nle-Arg-Pro-Arg-Gly-NH₂;
(SEQ ID NO: 78)
C20DA-gGlu₃-Sar₁₂-PipAc-Tyr-Phe-NmeLeu-Nle-Arg-Pro-Arg-Gly-NH₂;
(SEQ ID NO: 79)
C18DA-gGlu₃-Sar₁₅-PipAc-Tyr-Phe-NmeLeu-Nle-Dbu-NmeAla-Arg-Gly-NH₂;
and
(SEQ ID NO: 80)
C20DA-gGlu₃-Sar₁₅-PipAc-Tyr-Phe-NMeLeu-Nle-Orn-Pro-Arg-Gly-NH₂.

9. The compound of formula (I) according to claim 1 wherein the compound is in the form of a salt.

10. The compound of formula (I) according to claim 1, wherein the compound is part of a pharmaceutical composition together with at least one pharmaceutically acceptable adjuvant, diluent and/or carrier.

11. A method for preventing or treating a disease or condition associated with or modulated by Neuromedin U receptor 2 (NMUR2) activation, comprising administering to a patient in need thereof a therapeutically effective amount of a compound according to claim 1.

12. The method of claim 11, wherein the disease or condition associated with or modulated by NMUR2 activation is one or more of:
(i) inhibition, reduction or prevention of body weight gain, promotion of body weight reduction, chronic weight management;
(ii) prevention or treatment of body overweight condition;
(iii) prevention or treatment of obesity, morbid obesity, obesity-linked inflammation, obesity-linked gallbladder disease, or obesity related sleep apnea; and/or
(iv) prevention or treatment of conditions characterised by inadequate control of appetite, conditions characterised by over-feeding, binge-eating disorder, or Prader-Willi syndrome.

13. The method of claim 11, wherein the disease or condition associated with or modulated by NMUR2 activation is one or more of:
(v) lowering circulating LDL cholesterol levels, or increasing HDL/LDL ratio;
(vi) type 2 diabetes (T2D), impaired fasting glucose (IFG), or impaired glucose tolerance (IGT);
(vii) metabolic syndrome, hypertension, dyslipidemia, or atherogenic dyslipidemia,
(viii) chronic kidney diseases (CKD), hepatic steatosis, non-alcoholic fatty liver disease (NAFLD), or non-alcoholic steatohepatitis (NASH);
(ix) macrovascular disease, atherosclerosis, coronary heart disease, peripheral artery disease, stroke or microvascular disease;
(x) degeneration of cartilage, or osteoarthritis;
(xi) reproductive health complications of obesity or body overweight, or infertility;
(xii) cancer; and/or
(xiii) cognitive impairment, anxiety, or major depressive disorder;
preferably wherein said conditions, disorders or diseases are associated to a condition of body overweight or obesity.

14. The method of claim 11, wherein the compound is administered as part of a combination therapy together with at least one further active agent, wherein the at least one further active agent is for use in the treatment of obesity, diabetes, dyslipidemia or hypertension.

15. The method of claim 14, wherein the at least one further active agent for use in the treatment of obesity is selected from the group consisting of: a GIP receptor agonist, a GLP-1 receptor agonist, a GCG receptor agonist, oxyntomodulin or an oxyntomodulin analogue, a GCGR/GLP-1R dual agonist, a GLP-1R/GIPR dual agonist, a GLP-1R/GIPR/GCGR triple agonist, a peptide YY (PYY) or a PYY analogue, a neuropeptide Y (NPY) or an NPY analogue, an agonist for NPY2R, NPY4R or NPY5R, an antagonist or inverse agonist for NPY1R, a GLP-1R/NPY receptor dual agonist, a GLP-1R/NPY2R dual agonist, a GLP-1R/GIPR/NPYR triple agonist, a GLP-1R/GIPR/NPY2R triple agonist, a GLP-1R/GCGR/NPYR triple agonist, a GLP-1R/GCGR/NPY2R triple agonist, an amylin receptor agonist, and a GLP-1R/amylin receptor dual agonist.

16. The method according to claim 14, wherein the at least one further agent for use in the treatment of obesity is a GCGR/GLP-1R dual agonist of formula (III)
(III)
(SEQ ID NOs 87-88)
H-H-Ac4c-QGTFTSDYSKYLDERAAKDFI-K([17-carboxy-
heptadecanoyl]-gGlu-GSGSGG-)-WLESA-NH₂.

17. The method according to claim 14, wherein the at least one further agent for use in the treatment of obesity is semaglutide.

18. The compound according to claim 4, wherein m is 12 or 15.

19. The compound according to claim 5, wherein L is 2-[4-(2-aminoethyl)piperazin-1-yl]acetic acid (AEPipAc).

20. The compound according to claim 9, wherein the compound is in the form of a pharmaceutically acceptable salt.

21. The method according to claim 14, wherein the at least one further active agent is for use in the treatment of obesity.

22. The method according to claim 21, wherein the at least one further active agent for use in the treatment of obesity is selected from the group consisting of: semaglutide, danuglipron, GLP-1R agonist PF-07081532, tirzepatide, pemvidutide, GLP-1R/GIPR/GCGR triple agonist LY-3437943, cagrisema, amycretin, and combination semaglutide/PYY analogue NN-9775.


SEQUENCE LISTING (149 sequences)

SEQ ID NO: 1 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 27: note = Amine modified residue
EEEXXXXXXXXXXXXXXXXYFLXXPRG

SEQ ID NO: 2 - protein, 25 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 25: note = Amine modified residue
EEEEXXXXXXXXXXXXXYFLXRPRG

SEQ ID NO: 3 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 25: note = Ornithine
  SITE 28: note = Amine modified residue
EEEEXXXXXXXXXXXXXXXXYFLXXPRG

SEQ ID NO: 4 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 25: note = (2S)-2,4-diaminobutanoic acid
  SITE 28: note = Amine modified residue
EEEEXXXXXXXXXXXXXXXXYFLXXPRG

SEQ ID NO: 5 - protein, 25 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = Ornithine
  SITE 25: note = Amine modified residue
EEEEXXXXXXXXXXXXXYFLXXPRG

SEQ ID NO: 6 - protein, 24 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 24: note = Amine modified residue
EEEXXXXXXXXXXXXXYFLXRPRG

SEQ ID NO: 7 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXRPRG

SEQ ID NO: 8 - protein, 24 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-2,4-diaminobutanoic acid
  SITE 24: note = Amine modified residue
EEEXXXXXXXXXXXXXYFLXXPRG

SEQ ID NO: 9 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXRPRG

SEQ ID NO: 10 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 25: note = (2S)-N-methyl-alanine
  SITE 27: note = Amine modified residue
EEEXXXXXXXXXXXXXXXXYFLXRARG

SEQ ID NO: 11 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 25: note = (2S)-N-methyl-alanine
  SITE 27: note = Amine modified residue
EEEXXXXXXXXXXXXXXXXYFLXRARG

SEQ ID NO: 12 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 22: note = (2S)-N-methyl-alanine
  SITE 24: note = Amine modified residue
EEEXXXXXXXXXXXXXYFLXRARG

SEQ ID NO: 13 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = Ornithine
  SITE 23: note = (2S)-N-methyl-alanine
  SITE 25: note = Amine modified residue
EEEEXXXXXXXXXXXXXYFLXXARG

SEQ ID NO: 14 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 25: note = Ornithine
  SITE 26: note = (2S)-N-methyl-alanine
  SITE 28: note = Amine modified residue
EEEEXXXXXXXXXXXXXXXXYFLXXARG

SEQ ID NO: 15 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = (2S)-2,4-diaminobutanoic acid
  SITE 25: note = Amine modified residue
EEEEXXXXXXXXXXXXXYFLXXPRG

SEQ ID NO: 16 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 25: note = (2S)-2,4-diaminobutanoic acid
  SITE 26: note = (2S)-N-methyl-alanine
  SITE 28: note = Amine modified residue
EEEEXXXXXXXXXXXXXXXXYFLXXARG

SEQ ID NO: 17 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 28: note = Amine modified residue
EEEEXXXXXXXXXXXXXXXXYFLXKPRG

SEQ ID NO: 18 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  SITE 20: note = 2-[4-(2-aminoethyl)piperazin-1-yl]acetic acid
  SITE 23: note = (2S)-N-methyl-leucine
  SITE 24: note = Norleucine
  SITE 26: note = (2S)-N-methyl-alanine
  SITE 28: note = Amine modified residue
EEEEXXXXXXXXXXXXXXXXYFLXKARG

SEQ ID NO: 19 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 23: note = (2S)-N-methyl-alanine
  SITE 25: note = Amine modified residue
EEEEXXXXXXXXXXXXXYFLXKARG

SEQ ID NO: 20 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = Ornithine
  SITE 22: note = (2S)-N-methyl-alanine
  SITE 24: note = Amine modified residue
EEEXXXXXXXXXXXXXYFLXXARG

SEQ ID NO: 21 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 27: note = Amine modified residue
EEEXXXXXXXXXXXXXXXXYFLXXPRG

SEQ ID NO: 22 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 25: note = (2S)-N-methyl-alanine
  SITE 27: note = Amine modified residue
EEEXXXXXXXXXXXXXXXXYFLXXARG

SEQ ID NO: 23 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2,4-diaminobutanoic acid
  SITE 25: note = (2S)-N-methyl-alanine
  SITE 27: note = Amine modified residue
EEEXXXXXXXXXXXXXXXXYFLXXARG

SEQ ID NO: 24 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = (2S)-2,4-diaminobutanoic acid
  SITE 21: note = Amine modified residue
EEEXXXXXXXXXXYFLXXPRG

SEQ ID NO: 25 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 24: note = Amine modified residue
EEEXXXXXXXXXXXXXYFLXRPRG

SEQ ID NO: 26 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2,4-diaminobutanoic acid
  SITE 27: note = Amine modified residue
EEEXXXXXXXXXXXXXXXXYFLXXPRG

SEQ ID NO: 27 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-2,4-diaminobutanoic acid
  SITE 24: note = Amine modified residue
EEEXXXXXXXXXXXXXYFLXXPRG

SEQ ID NO: 28 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = (2S)-2,4-diaminobutanoic acid
  SITE 19: note = (2S)-N-methyl-alanine
  SITE 21: note = Amine modified residue
EEEXXXXXXXXXXYFLXXARG

SEQ ID NO: 29 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 27: note = Amine modified residue
EEEXXXXXXXXXXXXXXXXYFLXXGRG

SEQ ID NO: 30 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 25: note = Beta-alanine
  SITE 27: note = Amine modified residue
EEEXXXXXXXXXXXXXXXXYFLXXXRG

SEQ ID NO: 31 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = (2S)-2,4-diaminobutanoic acid
  SITE 23: note = (2S)-N-methyl-alanine
  SITE 25: note = Amine modified residue
EEEEXXXXXXXXXXXXXYFLXXARG

SEQ ID NO: 32 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 25: note = Amine modified residue
EEEEXXXXXXXXXXXXXYFLXKPRG

SEQ ID NO: 33 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = Ornithine
  SITE 21: note = Amine modified residue
EEEXXXXXXXXXXYFLXXPRG

SEQ ID NO: 34 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 25: note = (2S)-N-methyl-alanine
  SITE 27: note = Amine modified residue
EEEXXXXXXXXXXXXXXXXYFLXXARG

SEQ ID NO: 35 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-N-methyl-arginine
  SITE 24: note = Amine modified residue
EEEXXXXXXXXXXXXXYFLXRPRG

SEQ ID NO: 36 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-N-methyl-arginine
  SITE 25: note = (2S)-N-methyl-alanine
  SITE 27: note = Amine modified residue
EEEXXXXXXXXXXXXXXXXYFLXRARG

SEQ ID NO: 37 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-N-methyl-arginine
  SITE 22: note = (2S)-N-methyl-alanine
  SITE 24: note = Amine modified residue
EEEXXXXXXXXXXXXXYFLXRARG

SEQ ID NO: 38 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-2,4-diaminobutanoic acid
  SITE 22: note = (2S)-N-methyl-alanine
  SITE 24: note = Amine modified residue
EEEXXXXXXXXXXXXXYFLXXARG

SEQ ID NO: 39 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 25: note = Amine modified residue
EEEEXXXXXXXXXXXXXYFLXRPRG

SEQ ID NO: 40 - protein, 25 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = Ornithine
  SITE 25: note = Amine modified residue
EEEEXXXXXXXXXXXXXYFLXXPRG

SEQ ID NO: 41 - protein, 25 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = 2-[4-(2-aminoethyl)piperazin-1-yl]acetic acid
  SITE 20: note = (2S)-N-methyl-leucine
  SITE 21: note = Norleucine
  SITE 22: note = (2S)-2,4-diaminobutanoic acid
  SITE 25: note = Amine modified residue
EEEEXXXXXXXXXXXXXYFLXXPRG

SEQ ID NO: 42 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2,4-diaminobutanoic acid
  SITE 27: note = Amine modified residue
EEEXXXXXXXXXXXXXXXXYFLXXPRG

SEQ ID NO: 43 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2,4-diaminobutanoic acid
  SITE 25: note = (2S)-N-methyl-alanine
  SITE 27: note = Amine modified residue
EEEXXXXXXXXXXXXXXXXYFLXXARG

SEQ ID NO: 44 - protein, 20 aa, synthetic construct
  SITE 1: note = 17-carboxy-heptadecanoyl (S)-gamma-glutamic acid
  SITE 2: note = (S)-gamma-glutamic acid
  SITE 3: note = Sarcosine
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = 2-[4-(2-aminoethyl)piperazin-1-yl]acetic acid
  SITE 15: note = (2S)-N-methyl-leucine
  SITE 16: note = Norleucine
  SITE 17: note = Ornithine
  SITE 18: note = (2S)-N-methyl-alanine
  SITE 20: note = Amine modified residue
EEXXXXXXXXXXYFLXXARG

SEQ ID NO: 45 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-4-pyridyl-alanine
  SITE 27: note = Amine modified residue
EEEXXXXXXXXXXXXXXXXYFLXAPRG

SEQ ID NO: 46 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXKPRG

SEQ ID NO: 47 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 25: note = (2S)-N-methyl-alanine
  SITE 27: note = Amine modified residue
EEEXXXXXXXXXXXXXXXXYFLXKARG

SEQ ID NO: 48 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2-pyridyl-alanine
  SITE 25: note = (2S)-N-methyl-alanine
  SITE 27: note = Amine modified residue
EEEXXXXXXXXXXXXXXXXYFLXAARG

SEQ ID NO: 49 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXKPRG

SEQ ID NO: 50 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 25: note = (2S)-N-methyl-alanine
  SITE 27: note = Amine modified residue
EEEXXXXXXXXXXXXXXXXYFLXKARG

SEQ ID NO: 51 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXHPRG

SEQ ID NO: 52 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-N-methyl-arginine
  SITE 27: note = Amine modified residue
EEEXXXXXXXXXXXXXXXXYFLXRPRG

SEQ ID NO: 53 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-N-methyl-arginine
  SITE 25: note = (2S)-N-methyl-alanine
  SITE 27: note = Amine modified residue
EEEXXXXXXXXXXXXXXXXYFLXRARG

SEQ ID NO: 54 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-3-pyridyl-alanine
  SITE 27: note = Amine modified residue
EEEXXXXXXXXXXXXXXXXYFLXAPRG

SEQ ID NO: 55 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 25: note = (2S)-N-methyl-alanine
  SITE 27: note = Amine modified residue
EEEXXXXXXXXXXXXXXXXYFLXHARG

SEQ ID NO: 56 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-2-pyridyl-alanine
  SITE 27: note = Amine modified residue
EEEXXXXXXXXXXXXXXXXYFLXAPRG

SEQ ID NO: 57 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-3-pyridyl-alanine
  SITE 25: note = (2S)-N-methyl-alanine
  SITE 27: note = Amine modified residue
EEEXXXXXXXXXXXXXXXXYFLXAARG

SEQ ID NO: 58 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = (2S)-4-pyridyl-alanine
  SITE 25: note = (2S)-N-methyl-alanine
  SITE 27: note = Amine modified residue
EEEXXXXXXXXXXXXXXXXYFLXAARG

SEQ ID NO: 59 - protein, 24 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = (2S)-2,4-diaminobutanoic acid
  SITE 22: note = (2S)-N-methyl-alanine
  SITE 24: note = Amine modified residue
EEEXXXXXXXXXXXXXYFLXXARG

SEQ ID NO: 60 - protein, 21 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = (2S)-2,4-diaminobutanoic acid
  SITE 21: note = Amine modified residue
EEEXXXXXXXXXXYFLXXPRG

SEQ ID NO: 61 - protein, 21 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = (2S)-2,4-diaminobutanoic acid
  SITE 19: note = (2S)-N-methyl-alanine
  SITE 21: note = Amine modified residue
EEEXXXXXXXXXXYFLXXARG

SEQ ID NO: 62 - protein, 21 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 19: note = (2S)-N-methyl-alanine
  SITE 21: note = Amine modified residue
EEEXXXXXXXXXXYFLXRARG

SEQ ID NO: 63 - protein, 24 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 22: note = (2S)-N-methyl-alanine
  SITE 24: note = Amine modified residue
EEEXXXXXXXXXXXXXYFLXRARG

SEQ ID NO: 64 - skipped

SEQ ID NO: 65 - protein, 21 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 21: note = Amine modified residue
EEEXXXXXXXXXXYFLXRPRG

SEQ ID NO: 66 - protein, 20 aa, synthetic construct
  SITE 1: note = 17-carboxy-heptadecanoyl (S)-gamma-glutamic acid
  SITE 2: note = (S)-gamma-glutamic acid
  SITE 3: note = Sarcosine
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = 2-[4-(2-aminoethyl)piperazin-1-yl]acetic acid
  SITE 15: note = (2S)-N-methyl-leucine
  SITE 16: note = Norleucine
  SITE 18: note = (2S)-N-methyl-alanine
  SITE 20: note = Amine modified residue
EEXXXXXXXXXXYFLXRARG

SEQ ID NO: 67 - protein, 20 aa, synthetic construct
  SITE 1: note = 17-carboxy-heptadecanoyl (S)-gamma-glutamic acid
  SITE 2: note = (S)-gamma-glutamic acid
  SITE 3: note = Sarcosine
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = 2-[4-(2-aminoethyl)piperazin-1-yl]acetic acid
  SITE 15: note = (2S)-N-methyl-leucine
  SITE 16: note = Norleucine
  SITE 20: note = Amine modified residue
EEXXXXXXXXXXYFLXRPRG

SEQ ID NO: 68 - protein, 18 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = 2-[4-(2-aminoethyl)piperazin-1-yl]acetic acid
  SITE 13: note = (2S)-N-methyl-leucine
  SITE 14: note = Norleucine
  SITE 18: note = Amine modified residue
EEEXXXXXXXYFLXRPRG

SEQ ID NO: 69 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = Ornithine
  SITE 24: note = Amine modified residue
EEEXXXXXXXXXXXXXYFLXXPRG

SEQ ID NO: 70 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = Ornithine
  SITE 19: note = (2S)-N-methyl-alanine
  SITE 21: note = Amine modified residue
EEEXXXXXXXXXXYFLXXARG

SEQ ID NO: 71 - protein, 17 aa, synthetic construct
  SITE 1: note = 17-carboxy-heptadecanoyl (S)-gamma-glutamic acid
  SITE 2: note = (S)-gamma-glutamic acid
  SITE 3: note = Sarcosine
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = 2-[4-(2-aminoethyl)piperazin-1-yl]acetic acid
  SITE 12: note = (2S)-N-methyl-leucine
  SITE 13: note = Norleucine
  SITE 17: note = Amine modified residue
EEXXXXXXXYFLXRPRG

SEQ ID NO: 72 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 21: note = Amine modified residue
EEEXXXXXXXXXXYFLXRPRG

SEQ ID NO: 73 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 19: note = (2S)-N-methyl-alanine
  SITE 21: note = Amine modified residue
EEEXXXXXXXXXXYFLXRARG

SEQ ID NO: 74 - protein, 21 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = 2-[4-(2-aminoethyl)piperazin-1-yl]acetic acid
  SITE 16: note = (2S)-N-methyl-leucine
  SITE 17: note = Norleucine
  SITE 18: note = Homolysine
  SITE 21: note = Amine modified residue
EEEXXXXXXXXXXYFLXKPRG

SEQ ID NO: 75 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-(piperazin-1-yl)acetic acid
  SITE 22: note = (2S)-N-methyl-leucine
  SITE 23: note = Norleucine
  SITE 27: note = Amine modified residue
EEEXXXXXXXXXXXXXXXXYFLXRPRG

SEQ ID NO: 76 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-(piperazin-1-yl)acetic acid
  SITE 22: note = (2S)-N-methyl-leucine
  SITE 23: note = Norleucine
  SITE 24: note = (2S)-2,4-diaminobutanoic acid
  SITE 27: note = Amine modified residue
EEEXXXXXXXXXXXXXXXXYFLXXPRG

SEQ ID NO: 77 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-(piperazin-1-yl)acetic acid
  SITE 19: note = (2S)-N-methyl-leucine
  SITE 20: note = Norleucine
  SITE 24: note = Amine modified residue
EEEXXXXXXXXXXXXXYFLXRPRG

SEQ ID NO: 78 - protein, 24 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-(piperazin-1-yl)acetic acid
  SITE 19: note = (2S)-N-methyl-leucine
  SITE 20: note = Norleucine
  SITE 24: note = Amine modified residue
EEEXXXXXXXXXXXXXYFLXRPRG

SEQ ID NO: 79 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-(piperazin-1-yl)acetic acid
  SITE 22: note = (2S)-N-methyl-leucine
  SITE 23: note = Norleucine
  SITE 24: note = (2S)-2,4-diaminobutanoic acid
  SITE 25: note = (2S)-N-methyl-alanine
  SITE 27: note = Amine modified residue
EEEXXXXXXXXXXXXXXXXYFLXXARG

SEQ ID NO: 80 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-(piperazin-1-yl)acetic acid
  SITE 22: note = (2S)-N-methyl-leucine
  SITE 23: note = Norleucine
  SITE 24: note = Ornithine
  SITE 27: note = Amine modified residue
EEEXXXXXXXXXXXXXXXXYFLXXPRG

SEQ ID NO: 81 - protein, 27 aa, synthetic construct
  SITE 1: note = Octadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 27: note = Amine modified residue
EEEXXXXXXXXXXXXXXXXYFLXRPRG

SEQ ID NO: 82 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 23: note = Norleucine
  SITE 27: note = Amine modified residue
EEEXXXXXXXXXXXXXXXXYFLXRPRG

SEQ ID NO: 83 - protein, 27 aa, synthetic construct
  SITE 1: note = Icosanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 22: note = (2S)-N-methyl-leucine
  SITE 23: note = Norleucine
  SITE 24: note = Ornithine
  SITE 27: note = Amine modified residue
EEEXXXXXXXXXXXXXXXXYFLXXPRG

SEQ ID NO: 84 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-[4-(2-aminoethyl)piperazin-1-yl]acetic acid
  SITE 23: note = Norleucine
  SITE 24: note = Ornithine
  SITE 27: note = Amine modified residue
EEEXXXXXXXXXXXXXXXXYFLXXPRG

SEQ ID NO: 85 - protein, 24 aa, synthetic construct
  SITE 1: note = Octadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 19: note = (2S)-N-methyl-leucine
  SITE 20: note = Norleucine
  SITE 21: note = Ornithine
  SITE 24: note = Amine modified residue
EEEXXXXXXXXXXXXXYFLXXPRG

SEQ ID NO: 86 - protein, 24 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = 2-[4-(2-aminoethyl)piperazin-1-yl]acetic acid
  SITE 20: note = Norleucine
  SITE 21: note = Ornithine
  SITE 24: note = Amine modified residue
EEEXXXXXXXXXXXXXYFLXXPRG

SEQ ID NO: 87 - protein, 28 aa, synthetic construct
  SITE 1: note = 1-amino-cyclobutanecarboxylic acid
  SITE 24: note = Side chain of lysine linked via an amide bond to position 7 of SEQ ID NO: 88
  SITE 28: note = Amine modified residue
HQGTFTSDYSKYLDERAAKDFIKWLESA

SEQ ID NO: 88 - protein, 7 aa, synthetic construct
  SITE 1: note = 17-carboxy-heptadecanoyl (S)-gamma-glutamic acid
  SITE 7: note = Glycine is linked via an amide bond to position 24 of SEQ ID NO: 87
EGSGSGG

SEQ ID NO: 89 - protein, 25 aa, Homo sapiens
  SITE 25: note = Amine modified residue
FRVDEEFQSPFASQSRGYFLFRPRN

SEQ ID NO: 90 - protein, 8 aa, Homo sapiens
  SITE 8: note = Amine modified residue
YFLFRPRN

SEQ ID NO: 91 - protein, 8 aa, synthetic construct
  SITE 3: note = (2S)-N-methyl-leucine
  SITE 4: note = Norleucine
YFLXRPRG

SEQ ID NO: 92 - protein, 8 aa, synthetic construct
  SITE 3: note = (2S)-N-methyl-leucine
  SITE 4: note = Norleucine
  SITE 5: note = Ornithine
YFLXXPRG

SEQ ID NO: 93 - protein, 8 aa, synthetic construct
  SITE 3: note = (2S)-N-methyl-leucine
  SITE 4: note = Norleucine
  SITE 5: note = (2S)-2,4-diaminobutanoic acid
YFLXXPRG

SEQ ID NO: 94 - protein, 8 aa, synthetic construct
  SITE 3: note = (2S)-N-methyl-leucine
  SITE 4: note = Norleucine
  SITE 6: note = (2S)-N-methyl-alanine
YFLXRARG

SEQ ID NO: 95 - protein, 8 aa, synthetic construct
  SITE 3: note = (2S)-N-methyl-leucine
  SITE 4: note = Norleucine
  SITE 5: note = Ornithine
  SITE 6: note = (2S)-N-methyl-alanine
YFLXXARG

SEQ ID NO: 96 - protein, 8 aa, synthetic construct
  SITE 3: note = (2S)-N-methyl-leucine
  SITE 4: note = Norleucine
  SITE 5: note = (2S)-2,4-diaminobutanoic acid
  SITE 6: note = (2S)-N-methyl-alanine
YFLXXARG

SEQ ID NO: 97 - protein, 8 aa, synthetic construct
  SITE 3: note = (2S)-N-methyl-leucine
  SITE 4: note = Norleucine
YFLXKPRG

SEQ ID NO: 98 - protein, 8 aa, synthetic construct
  SITE 3: note = (2S)-N-methyl-leucine
  SITE 4: note = Norleucine
  SITE 6: note = (2S)-N-methyl-alanine
YFLXKARG

SEQ ID NO: 99 - protein, 8 aa, synthetic construct
  SITE 3: note = (2S)-N-methyl-leucine
  SITE 4: note = Norleucine
  SITE 5: note = Ornithine
YFLXXGRG

SEQ ID NO: 100 - protein, 8 aa, synthetic construct
  SITE 3: note = (2S)-N-methyl-leucine
  SITE 4: note = Norleucine
  SITE 5: note = Ornithine
  SITE 6: note = Beta-alanine
YFLXXXRG

SEQ ID NO: 101 - protein, 8 aa, synthetic construct
  SITE 3: note = (2S)-N-methyl-leucine
  SITE 4: note = Norleucine
  SITE 5: note = (2S)-N-methyl-arginine
  SITE 6: note = (2S)-N-methyl-alanine
YFLXRARG

SEQ ID NO: 102 - protein, 8 aa, synthetic construct
  SITE 3: note = (2S)-N-methyl-leucine
  SITE 4: note = Norleucine
  SITE 5: note = (2S)-4-pyridyl-alanine
YFLXAPRG

SEQ ID NO: 103 - protein, 8 aa, synthetic construct
  SITE 3: note = (2S)-N-methyl-leucine
  SITE 4: note = Norleucine
  SITE 5: note = (2S)-2-pyridyl-alanine
  SITE 6: note = (2S)-N-methyl-alanine
YFLXAARG

SEQ ID NO: 104 - protein, 8 aa, synthetic construct
  SITE 3: note = (2S)-N-methyl-leucine
  SITE 4: note = Norleucine
YFLXHPRG

SEQ ID NO: 105 - protein, 8 aa, synthetic construct
  SITE 3: note = (2S)-N-methyl-leucine
  SITE 4: note = Norleucine
  SITE 5: note = (2S)-N-methyl-arginine
YFLXRPRG

SEQ ID NO: 106 - protein, 8 aa, synthetic construct
  SITE 3: note = (2S)-N-methyl-leucine
  SITE 4: note = Norleucine
  SITE 5: note = (2S)-3-pyridyl-alanine
YFLXAPRG

SEQ ID NO: 107 - protein, 8 aa, synthetic construct
  SITE 3: note = (2S)-N-methyl-leucine
  SITE 4: note = Norleucine
  SITE 6: note = (2S)-N-methyl-alanine
YFLXHARG

SEQ ID NO: 108 - protein, 8 aa, synthetic construct
  SITE 3: note = (2S)-N-methyl-leucine
  SITE 4: note = Norleucine
  SITE 5: note = (2S)-2-pyridyl-alanine
YFLXAPRG

SEQ ID NO: 109 - protein, 8 aa, synthetic construct
  SITE 3: note = (2S)-N-methyl-leucine
  SITE 4: note = Norleucine
  SITE 5: note = (2S)-3-pyridyl-alanine
  SITE 6: note = (2S)-N-methyl-alanine
YFLXAARG

SEQ ID NO: 110 - protein, 8 aa, synthetic construct
  SITE 3: note = (2S)-N-methyl-leucine
  SITE 4: note = Norleucine
  SITE 5: note = (2S)-4-pyridyl-alanine
  SITE 6: note = (2S)-N-methyl-alanine
YFLXAARG

SEQ ID NO: 111 - protein, 8 aa, synthetic construct
  SITE 3: note = (2S)-N-methyl-leucine
  SITE 4: note = Norleucine
  SITE 5: note = Homolysine
YFLXKPRG

SEQ ID NO: 112 - protein, 8 aa, synthetic construct
  SITE 3: note = (2S)-N-methyl-leucine
  SITE 4: note = Norleucine
  VARIANT 5: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 6: note = Proline, (2S)-N-methyl-alanine, Beta-alanine, or Glycine
YFLXXXRG

SEQ ID NO: 113 - protein, 8 aa, synthetic construct
  SITE 3: note = (2S)-N-methyl-leucine
  SITE 4: note = Norleucine
  VARIANT 5: note = Arginine, (2S)-2,4-diaminobutanoic acid, (2S)-N-methyl-arginine, Ornithine, or Lysine
  VARIANT 6: note = Proline or (2S)-N-methyl-alanine
YFLXXXRG

SEQ ID NO: 114 - protein, 8 aa, synthetic construct
  SITE 3: note = (2S)-N-methyl-leucine
  SITE 4: note = Norleucine
  VARIANT 5: note = Arginine, (2S)-2,4-diaminobutanoic acid, or Ornithine
YFLXXPRG

SEQ ID NO: 115 - protein, 19 aa, synthetic construct
  SITE 1: note = (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2, or 3 residues deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3, 6, 9, or 12 residues deleted
EEEEXXXXXXXXXXXXXXX

SEQ ID NO: 116 - protein, 19 aa, synthetic construct
  SITE 1: note = (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
EEEEXXXXXXXXXXXXXXX

SEQ ID NO: 117 - skipped

SEQ ID NO: 118 - protein, 19 aa, synthetic construct
  SITE 1: note = (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 residues deleted
EEEEXXXXXXXXXXXXXXX

SEQ ID NO: 119 - protein, 28 aa, synthetic construct
  VARIANT 1: note = 15-carboxy-pentadecanoyl, 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2, or 3 residues deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 20: note = 2-[4-(2-aminoethyl)piperazin-1-yl]acetic acid, 2-(piperazin-1-yl)acetic acid or 2-[1-(2-aminoethyl)piperidin-4-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
  VARIANT 5..19: note = This region may have 3, 6, 9, or 12 residues deleted
EEEEXXXXXXXXXXXXXXXXYFLXXXRG

SEQ ID NO: 120 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2, or 3 residues deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3, 6, 9, or 12 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
EEEEXXXXXXXXXXXXXXXXYFLXXXRG

SEQ ID NO: 121 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 122 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 123 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  SITE 19: note = 2-[4-(2-aminoethyl)piperazin-1-yl]acetic acid
  SITE 22: note = (2S)-N-methyl-leucine
  SITE 23: note = Norleucine
  VARIANT 24: note = Arginine, (2S)-2,4-diaminobutanoic acid, or Ornithine
  SITE 27: note = Amine modified residue
EEEXXXXXXXXXXXXXXXXYFLXXPRG

SEQ ID NO: 124 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 125 - protein, 19 aa, synthetic construct
  SITE 1: note = (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
EEEEXXXXXXXXXXXXXXX

SEQ ID NO: 126 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 127 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 128 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = 2-(piperazin-1-yl)acetic acid
  SITE 23: note = Norleucine
  SITE 24: note = Ornithine
  SITE 27: note = Amine modified residue
EEEXXXXXXXXXXXXXXXXYFLXXPRG

SEQ ID NO: 129 - protein, 8 aa, synthetic construct
  SITE 3: note = (2S)-N-methyl-leucine
  SITE 4: note = Norleucine
  VARIANT 5: note = Any amino acid
  VARIANT 6: note = Any amino acid
YFLXXXRG

SEQ ID NO: 130 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2, or 3 residues deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3, 6, 9, or 12 residues deleted
  VARIANT 20: note = 2-[4-(2-aminoethyl)piperazin-1-yl]acetic acid, 2-(piperazin-1-yl)acetic acid or 2-[1-(2-aminoethyl)piperidin-4-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
EEEEXXXXXXXXXXXXXXXXYFLXXXRG

SEQ ID NO: 131 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2 or 3 residues deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3, 6, 9, or 12 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
EEEEXXXXXXXXXXXXXXXXYFLXXXRG

SEQ ID NO: 132 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
EEEEXXXXXXXXXXXXXXXXYFLXXXRG

SEQ ID NO: 133 - protein, 28 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, Lysine, Histidine, Homolysine, (2S)-2-pyridyl-alanine, (2S)-3-pyridyl-alanine, or (2S)-4-pyridyl-alanine
  VARIANT 26: note = Proline, (2S)-N-methyl-alanine, Beta-alanine, or Glycine
  SITE 28: note = Amine modified residue
EEEEXXXXXXXXXXXXXXXXYFLXXXRG

SEQ ID NO: 134 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 135 - protein, 27 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  VARIANT 19: note = 2-[4-(2-aminoethyl)piperazin-1-yl]acetic acid or 2-(piperazin-1-yl)acetic acid
  SITE 22: note = (2S)-N-methyl-leucine
  SITE 23: note = Norleucine
  VARIANT 24: note = Arginine, (2S)-2,4-diaminobutanoic acid, (2S)-N-methyl-arginine, Ornithine, or Lysine
  VARIANT 25: note = Proline or (2S)-N-methyl-alanine
  SITE 27: note = Amine modified residue
EEEXXXXXXXXXXXXXXXXYFLXXXRG

SEQ ID NO: 136 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 137 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 138 - protein, 27 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  VARIANT 19: note = 2-[4-(2-aminoethyl)piperazin-1-yl]acetic acid or 2-(piperazin-1-yl)acetic acid
  SITE 22: note = (2S)-N-methyl-leucine
  SITE 23: note = Norleucine
  VARIANT 24: note = Arginine, (2S)-2,4-diaminobutanoic acid, or Ornithine
  SITE 27: note = Amine modified residue
EEEXXXXXXXXXXXXXXXXYFLXXPRG

SEQ ID NO: 139 - protein, 28 aa, synthetic construct
  VARIANT 1: note = 17-carboxy-heptadecanoyl or 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 140 - protein, 28 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 141 - protein, 28 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 142 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  VARIANT 19: note = 2-[4-(2-aminoethyl)piperazin-1-yl]acetic acid or 2-(piperazin-1-yl)acetic acid
  SITE 22: note = (2S)-N-methyl-leucine
  SITE 23: note = Norleucine
  VARIANT 24: note = Arginine, (2S)-2,4-diaminobutanoic acid, (2S)-N-methyl-arginine, Ornithine, or Lysine
  VARIANT 25: note = Proline or (2S)-N-methyl-alanine
  SITE 27: note = Amine modified residue
EEEXXXXXXXXXXXXXXXXYFLXXXRG

SEQ ID NO: 143 - protein, 28 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, or Ornithine
  SITE 28: note = Amine modified residue
EEEEXXXXXXXXXXXXXXXXYFLXXPRG

SEQ ID NO: 144 - protein, 27 aa, synthetic construct
  SITE 1: note = 17-carboxy-heptadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  VARIANT 19: note = 2-[4-(2-aminoethyl)piperazin-1-yl]acetic acid or 2-(piperazin-1-yl)acetic acid
  SITE 22: note = (2S)-N-methyl-leucine
  SITE 23: note = Norleucine
  VARIANT 24: note = Arginine, (2S)-2,4-diaminobutanoic acid, or Ornithine
  SITE 27: note = Amine modified residue
EEEXXXXXXXXXXXXXXXXYFLXXPRG

SEQ ID NO: 145 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  VARIANT 20: note = 2-[4-(2-aminoethyl)piperazin-1-yl]acetic acid or 2-(piperazin-1-yl)acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 146 - protein, 28 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1 residue deleted
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  SITE 19: note = Sarcosine
  VARIANT 5..19: note = This region may have 3 or 6 residues deleted
  SITE 20: note = 2-[4-(2-aminoethyl)piperazin-1-yl]acetic acid
  SITE 23: note = (2S)-N-methyl-leucine
  SITE 24: note = Norleucine
  VARIANT 25: note = Arginine, (2S)-2,4-diaminobutanoic acid, (2S)-N-methyl-arginine, Ornithine, or Lysine
  VARIANT 26: note = Proline or (2S)-N-methyl-alanine
  SITE 28: note = Amine modified residue
EEEEXXXXXXXXXXXXXXXXYFLXXXRG

SEQ ID NO: 147 - protein, 27 aa, synthetic construct
  SITE 1: note = 19-carboxy-nonadecanoyl (S)-gamma-glutamic acid
  SITE 2: note = (S)-gamma-glutamic acid
  SITE 3: note = (S)-gamma-glutamic acid
  SITE 4: note = Sarcosine
  SITE 5: note = Sarcosine
  SITE 6: note = Sarcosine
  SITE 7: note = Sarcosine
  SITE 8: note = Sarcosine
  SITE 9: note = Sarcosine
  SITE 10: note = Sarcosine
  SITE 11: note = Sarcosine
  SITE 12: note = Sarcosine
  SITE 13: note = Sarcosine
  SITE 14: note = Sarcosine
  SITE 15: note = Sarcosine
  SITE 16: note = Sarcosine
  SITE 17: note = Sarcosine
  SITE 18: note = Sarcosine
  VARIANT 4..18: note = This region may have 3 residues deleted
  VARIANT 19: note = 2-[4-(2-aminoethyl)piperazin-1-yl]acetic acid or 2-(piperazin-1-yl)acetic acid
  SITE 22: note = (2S)-N-methyl-leucine
  SITE 23: note = Norleucine
  VARIANT 24: note = Arginine, (2S)-2,4-diaminobutanoic acid, (2S)-N-methyl-arginine, Ornithine, or Lysine
  VARIANT 25: note = Proline or (2S)-N-methyl-alanine
  SITE 27: note = Amine modified residue
EEEXXXXXXXXXXXXXXXXYFLXXXRG

SEQ ID NO: 148 - protein, 8 aa, synthetic construct
  SITE 3: note = (2S)-N-methyl-leucine
  SITE 4: note = Norleucine
  VARIANT 5: note = Arginine, (2S)-2,4-diaminobutanoic acid, (2S)-N-methyl-arginine, Ornithine, or Lysine
  VARIANT 6: note = Proline or (2S)-N-methyl-alanine
YFLXXXRG

SEQ ID NO: 149 - protein, 19 aa, synthetic construct
  SITE 1: note = (S)-gamma-glutamic acid
  SITE 2: note = (S)-gamma-glutamic acid
  SITE 3: note = (S)-gamma-glutamic acid
  SITE 4: note = (S)-gamma-glutamic acid
  VARIANT 1..4: note = This region may have 1, 2 or 3 residues deleted
  SITE 5: note = sarcosine
  SITE 6: note = sarcosine
  SITE 7: note = sarcosine
  SITE 8: note = sarcosine
  SITE 9: note = sarcosine
  SITE 10: note = sarcosine
  SITE 11: note = sarcosine
  SITE 12: note = sarcosine
  SITE 13: note = sarcosine
  SITE 14: note = sarcosine
  SITE 15: note = sarcosine
  SITE 16: note = sarcosine
  SITE 17: note = sarcosine
  SITE 18: note = sarcosine
  SITE 19: note = sarcosine
  VARIANT 5..19: note = This region may have 3, 4, 5, 6, 7, 8, 9, 10, 11, or 12 residues deleted
EEEEXXXXXXXXXXXXXXX